(19) United States
(12) Patent Application Publication
(10) Pub. No.: US 2011/0218515 A1
(43) Pub. Date: Sep. 8, 2011

(54) Methods for Treating Acute Myocardial Infarctions and Associated Disorders

(75) Inventor: Jeff Olgin, Larkspur, CA (US)

(73) Assignee: The Regents of the University of California, Oakland, CA (US)

(21) Appl. No.: 13/115,697

(22) Filed: May 25, 2011

Related U.S. Application Data
(63) Continuation of application No. 12/693,906, filed on Jan. 26, 2010.
(60) Provisional application No. 61/147,340, filed on Jan. 26, 2009.

(51) Int. Cl. A61M 31/00

(52) U.S. Cl. 604/500

(57) ABSTRACT

The invention relates to methods of treating patients who have suffered an acute myocardial infarction (AMI) with a therapeutic that has anti-fibrotic effects, for example, pirfenidone and analogs thereof.

### CROSS-REFERENCE TO RELATED APPLICATIONS

This application is a continuation of U.S. application Ser. No. 12/693,906, filed Jan. 26, 2010 which claims the benefit of U.S. Provisional Application No. 61/147,340, filed Jan. 26, 2009, the disclosure of which is incorporated herein by reference in its entirety.

### FIELD OF THE INVENTION

The invention relates to methods of treating patients who have suffered an acute myocardial infarction (AMI) and associated disorders with a therapeutic that has anti-fibrotic effects, for example, pirfenidone and analogs thereof.

### BACKGROUND

There are approximately 1.5 million cases of acute myocardial infarction (AMI) in the United States each year, resulting in more than 500,000 deaths. Many of the deaths resulting from AMI occur before the patient can reach the hospital. Despite medical and interventional advances in the treatment of acute coronary syndromes over the last two decades, patients continue to face significant morbidity and mortality following a myocardial infarction. Post-myocardial infarction (MI) complications include congestive heart failure (CHF) and ventricular tachycardia (VT).

Contraction of the heart is initiated by an electrical impulse generated by the sinoatrial node, a natural pacemaker, in the heart. The heart's electrical conduction system then conveys the impulse to the myocardium, or cardiac muscle, to stimulate contraction. Abnormal electrical conduction due to structural tissue remodeling after infarction may play an important role in ventricular arrhythmias, which can lead to sudden cardiac arrest and death. Tissue remodeling is due in part to direct tissue damage, neurohormonal activation, cytokine release, inflammation and fibrosis.

Medical therapeutics, including drug therapy aimed at suppressing and preventing ventricular arrhythmias have thus far been disappointing. Earlier agents, including class IC anti-arrhythmics, were unexpectedly pro-arrhythmic in the setting of coronary artery disease and raised a cautionary note. Current post-MI pharmacotherapies include renin-angiotensin-aldosterone (RAA) blockers, which improve cardiac remodeling but do not specifically target fibrosis. It is an object of the present invention to provide novel therapies and therapeutic regimens for treating acute myocardial infarction.

### SUMMARY

It has been unexpectedly found that a compound which inhibits fibrosis has beneficial effects on left ventricular (LV) function, infarct size, peri-infarct fibrosis, electrophysiology of the infarct border zone and VT inducibility. It is also unexpected that such compounds offer a more targeted and effective inhibition of detrimental post-acute MI remodeling than RAA blockers. Provided herein are novel means to prevent arrhythmias in the post-acute MI period, and to improve heart contractility, improve heart function and reduce complications of acute MI such as congestive heart failure (CHF) and ventricular tachycardia (VT) and ventricular fibrillation.

Without being bound by a theory of the invention, early fibrosis in response to cardiac injury is believed to be important in forming a healing scar and serves as a compensatory function in preventing infarct expansion, aneurysm formation, and cardiac perforation. However, late-onset and excessive fibrosis beyond the infarct, and into the infarct border zone and other viable tissues, can contribute to adverse cardiac remodeling. Cardiac fibrosis can cause altered propagation, leading to non-uniform anisotropic conduction that eventually causes the formation of re-entry circuits and potentially wave breaks that predispose to arrhythmogenesis. The results described herein indicate that inhibiting late-onset fibrosis can provide measurable beneficial effects in the post-acute MI setting.

In the broadest feature, the present invention discloses a method of treating a patient who has suffered a myocardial infarction (MI), or who has not previously suffered an MI, or is within a week of suffering an MI, comprising administering to the patient a therapeutically effective dose of a therapeutic having an anti-fibrotic effect. In another aspect, the present invention discloses a method of treating a patient who has suffered a myocardial infarction (e.g. an acute myocardial infarction (AMI)) comprising administering to the patient a therapeutically effective dose of a therapeutic having an anti-fibrotic effect, wherein optionally the treatment is initiated immediately after suffering the myocardial infarction (e.g. the AMI), and optionally continues for up to 3 to 6 months. In some aspects, the method is to limit expansion of an infarct scar due to the myocardial infarction (e.g. the AMI).

In another aspect, the invention provides a method of treating a patient who has suffered myocardial infarction (e.g. an AMI) comprising administering to said patient a therapeutically effective dose of a therapeutic having an anti-fibrotic effect. In some embodiments, the treatment is initiated at a time period about 1 to 42 days after suffering the myocardial infarction (e.g. the AMI), and optionally continues for up to 3 to 6 months. In other embodiments, the treatment is initiated at a time period about 3 to 14 days after suffering the myocardial infarction (e.g. the AMI), and optionally continues for up to 3 to 6 months. In another embodiment, the treatment is initiated about 5-10 days after the myocardial infarction (e.g. the AMI). In another embodiment, the treatment is initiated about 2-40 days after the myocardial infarction (e.g. the AMI). In another embodiment, the treatment is initiated about 3-20 days after the myocardial infarction (e.g. the AMI). In another embodiment, the treatment is initiated about 4-15 days after the myocardial infarction (e.g. the AMI). In yet another embodiment, the treatment is initiated about 7 days after the myocardial infarction (e.g. the AMI). In some embodiments, the treatment continues for a period of at least 2 weeks. In other embodiments, the treatment after being initiated continues for a time period until about 4 weeks after the myocardial infarction (e.g. the AMI). Thus, the invention encompasses treatment of patients from about 14 days to 4 weeks after the myocardial infarction (e.g. the AMI).

In an embodiment, the invention provides a method of reducing the incidence of congestive heart failure (CHF) in a patient who suffered a myocardial infarction (e.g., an acute myocardial infarction (AMI)), comprising administering to said patient a therapeutically effective dose of a therapeutic having an anti-fibrotic effect, wherein the therapeutically effective dose reduces the incidence of congestive heart failure, and wherein optionally the treatment is initiated at a time period about 1 to 42 days after suffering the myocardial infarction (e.g. the AMI). In some aspects, the patient is at an increased risk of congestive heart failure due to the myocardial infarction (e.g. the AMI).

In an embodiment, the invention provides a method of preserving viable cardiac tissue or controlling myocardial infarct size in a patient who has suffered a myocardial infarction (e.g. an acute myocardial infarction (AMI)) comprising administering to said patient a therapeutically effective dose of a therapeutic having an anti-fibrotic effect, wherein the administering of said therapeutic to said patient results in a relatively reduced infarct size on average compared to infarct size in a patient who has not been administered said therapeutic. In some embodiments, the treatment is initiated at a time period about 1 to 42 days after suffering the myocardial infarction (e.g. the AMI). In further embodiments, the relative reduction in infarct size is at least 5%.

In an embodiment, the invention provides a method of reducing the incidence of ventricular tachycardia in a patient in need thereof, comprising administering to said patient a therapeutically effective dose of a therapeutic having an anti-fibrotic effect. In some embodiments, the patient has suffered a myocardial infarction (e.g. an AMI). In further embodiments, the treatment is initiated at a time period about 1 to 42 days after suffering the myocardial infarction (e.g. the AMI). In another embodiment, the administering is initiated about 7 days after suffering the myocardial infarction (e.g. the AMI).

In an embodiment, the invention provides a method of treating or preventing ventricular fibrillation in a patient in need thereof is provided, comprising administering to said patient a therapeutic having an anti-fibrotic effect. In some embodiments, the patient has suffered a myocardial infarction (e.g. an AMI). In further embodiments, the treatment is initiated at a time period about 1 to 42 days after suffering the myocardial infarction (e.g. the AMI). In another embodiment, the administering is initiated about 7 days after the suffering of the myocardial infarction (e.g. the AMI). In another embodiment, the administering reduces the incidence of sudden cardiac death relative to the incidence of cardiac death in the absence of administration of the therapeutic. In still another embodiment, the administering reduces cardiac risk of the patient relative to the cardiac risk in the absence of administration of the therapeutic. As used herein, the term “cardiac risk” means the risk of cardiac morbidity resulting from any one or a combination of ventricular tachycardia, sudden cardiac death, ventricular fibrillation and/or congestive heart failure.

In some embodiments, the invention provides a method of controlling (e.g., reduce, reduce the incidence or severity of, or prevent the progression of) arrhythmia in a patient in need thereof is provided, comprising administering to the patient a therapeutic having an anti-fibrotic effect, wherein the administering of the therapeutic controls (e.g., reduce, reduce the incidence or severity of, or prevent the progression of) arrhythmia in the patient. In some embodiments, the administering reduces the incidence or severity of arrhythmia in the patient relative to the incidence or severity of arrhythmia in the absence of administration of the therapeutic. In some embodiments the patient has suffered a myocardial infarction (e.g. an AMI). In further embodiments the administration is initiated about 1 to 42 days after the suffering of the myocardial infarction (e.g. the AMI). In still further embodiments the administration is initiated about 7 days after the suffering of the myocardial infarction (e.g. the AMI). In other embodiments, the administering treats ventricular remodeling.

In some embodiments of any of the preceding methods, the patient is diagnosed as suffering a first myocardial infarction (e.g. a first AMI), i.e. the patient has not been diagnosed as having previously suffered a myocardial infarction (e.g. an AMI) or the patient has not previously suffered a myocardial infarction (e.g. an AMI). In some embodiments, any of the methods described herein optionally exclude treatment of patients diagnosed with chronic MI.

In some embodiments of any of the preceding methods, the therapeutic having an anti-fibrotic effect reduces tissue remodeling or fibrosis. In some embodiments of any of the preceding methods, the therapeutic having an anti-fibrotic effect reduces the activity of transforming growth factor-beta (TGF-β), targets one or more TGF-β isoforms, inhibits TGF-β receptor kinases TGFBR1 (ALK5) and/or TGFBR2, or modulates one or more post-receptor signaling pathways. In such cases, the therapeutically effective amount of such a compound may exhibit one or more of the foregoing effects in the TGF-β pathway and/or reduce fibrosis.

In some embodiments of any of the preceding methods, the therapeutic having an anti-fibrotic effect is an endothelin receptor antagonist, targets both endothelin receptor A and endothelin receptor B or selectively targets endothelin receptor A. In such cases, the therapeutically effective amount of such a compound may exhibit one or more of the foregoing effects in the endothelin A and/or B pathway, and/or reduce fibrosis.

In other embodiments of any of the preceding methods, the therapeutic having an anti-fibrotic effect reduces activity of connective tissue growth factor (CTGF). In such cases, the therapeutically effective amount of such a compound may exhibit one or more of the foregoing effects in the CTGF pathway and/or reduce fibrosis.

In further embodiments of any of the preceding methods, the therapeutic having an anti-fibrotic effect inhibits matrix metalloproteinase (MMP). In such cases, the therapeutically effective amount of such a compound may inhibit MMP and/or reduce fibrosis. In certain embodiments, the therapeutically effective amount of such a compound may inhibit MMP-9 or MMP-12.

In still other embodiments of any of the preceding methods, the therapeutic having an anti-fibrotic effect reduces the activity of epidermal growth factor receptor (4), targets EGF receptor, or inhibits EGF receptor kinase. In such cases, the therapeutically effective amount of such a compound may exhibit one or more of the foregoing effects in the EGF pathway and/or reduce fibrosis.

In some embodiments of any of the preceding methods, the therapeutic having an anti-fibrotic effect reduces the activity of platelet derived growth factor (PDGF), targets PDGF receptor (PDGFR), inhibits PDGFR kinase activity, or inhibits post-PDGF receptor signaling pathways. In such cases, the therapeutically effective amount of such a compound may exhibit one or more of the foregoing effects in the PDGF pathway and/or reduce fibrosis.

In some embodiments of any of the preceding methods, the therapeutic having an anti-fibrotic effect reduces the activity of vascular endothelial growth factor (VEGF), targets one or more of VEGF, VEGF receptor 1 (VEGFR1, Flt-1), or VEGF receptor 2 (VEGFR2, KDR). In such cases, the therapeutically effective amount of such a compound may exhibit one or more of the foregoing effects in the VEGF pathway and/or reduce fibrosis.

In other embodiments of any of the preceding methods, the therapeutic having an anti-fibrotic effect inhibits multiple receptor kinases such as BIRB-1120 which inhibits receptor kinases for vascular endothelial growth factor, fibroblast growth factor, and platelet derived growth factor. In such cases, the therapeutically effective amount of such a compound may inhibit one or more receptor kinases in the VEGF, FGF or PDGF pathways and/or reduce fibrosis.

In some embodiments of any of the preceding methods, the therapeutic having an anti-fibrotic effect interferes with integrin function. In such cases, the therapeutically effective amount of such a compound may inhibit integrin function and/or reduce fibrosis. In further embodiments of any of the preceding methods, the therapeutic having an anti-fibrotic effect may inhibit αV integrins. In other embodiments, the therapeutic having an anti-fibrotic effect may inhibit integrin αVβ6 function.

In some embodiments of any of the preceding methods, the therapeutic having an anti-fibrotic effect interferes with pro-fibrotic activities of IL-4 and IL-13, targets IL-4 receptor, IL-13 receptor. In such cases, the therapeutically effective amount of such a compound may exhibit one or more of the foregoing effects in the IL-4 and/or IL-13 pathway and/or reduce fibrosis.

In further embodiments of any of the preceding methods, the therapeutic having an anti-fibrotic effect modulates signaling through the JAK-STAT pathway. In such cases, the therapeutically effective amount of such a compound may modulate signaling through the JAK-STAT pathway and/or reduce fibrosis.

In some embodiments of any of the preceding methods, the therapeutic having an anti-fibrotic effect interferes with epithelial mesenchymal transition, or inhibits mTor. In such cases, the therapeutically effective amount of such a compound may exhibit one or more of the foregoing effects on mesenchyma, and/or reduce fibrosis.

In some embodiments of any of the preceding methods, the therapeutic having an anti-fibrotic effect reduces levels of copper. In such cases, the therapeutically effective amount of such a compound may reduce copper levels in circulation and/or tissue, and/or reduce fibrosis.

In some embodiments of any of the preceding methods, the therapeutic having an anti-fibrotic effect reduces oxidative stress. In such cases, the therapeutically effective amount of such a compound may reduce oxidative stress and/or reduce fibrosis.

In still further embodiments of any of the preceding methods, the therapeutic having an anti-fibrotic effect inhibits prolyl hydrolyse. In such cases, the therapeutically effective amount of such a compound may reduce prolyl hydrolase and/or reduce fibrosis.

In some embodiments of any of the preceding methods, the therapeutic having an anti-fibrotic effect is an agonist of proliferator-activated receptor-gamma (PPAR-γ).

In some embodiments of any of the preceding methods, the therapeutic having an anti-fibrotic effect inhibits phosphodiesterase 4 (PDE4) or phosphodiesterase 5 (PDE5), or modifies the arachidonic acid pathway. In such cases, the therapeutically effective amount of such a compound may inhibit the PDE4 and/or PDE5 pathway, or may inhibit the arachidonic acid pathway, and/or reduce fibrosis.

In various embodiments of any of the preceding methods, the therapeutic having an anti-fibrotic effect is combined with a pharmaceutically acceptable carrier. In other embodiments of any of the preceding methods, the administration is oral.

In some embodiments of any of the preceding methods, the therapeutically effective amount is a total daily dose of about 50 mg to about 2400 mg of said therapeutic or a pharmaceutically acceptable salt, ester, solvate, or prodrug thereof.

In some embodiments of any of the preceding methods, the therapeutically effective amount is administered in divided doses three times a day or two times a day, or is administered in a single dose once a day.

In various embodiments of any of the preceding methods, said therapeutic is pirfenidone or compound of formula (I), (II), (III), (IV), or (V) or a pharmaceutically acceptable salt, ester, solvate, or prodrug thereof:

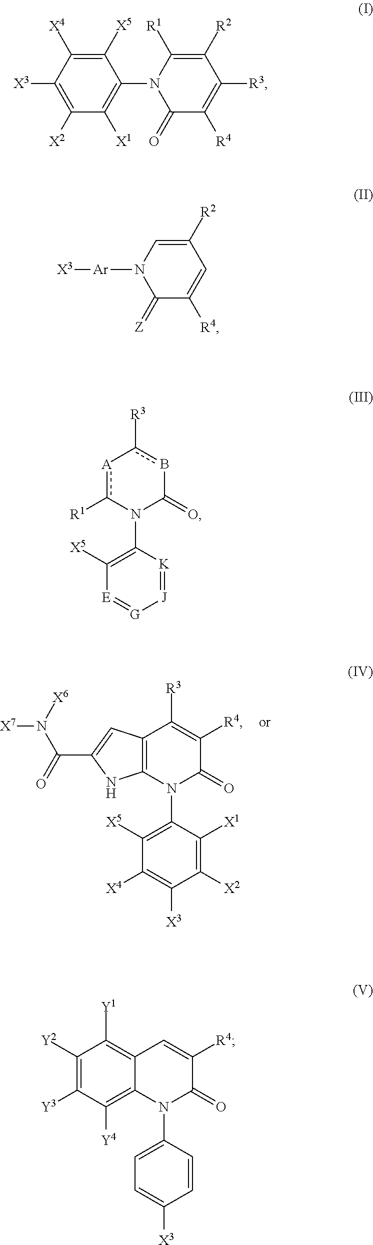

wherein

A is N or CR²; B is N or CR⁴; E is N or CX⁴; G is N or CX³; J is N or CX²; K is N or CX¹; a dashed line is a single or double bond,

R¹, R², R³, R⁴, X¹, X², X³, X⁴, X⁵, Y¹, Y², Y³, and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted C₁-C₁₀ alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, substituted alkoxyalkyl, C₁-C₁₀ carboxy, substituted C₁-C₁₀ carboxy, C₁-C₁₀ alkoxycarbonyl, substituted alkoxycarbonyl, CO-uronide, CO-monosaccharide, CO-oligosaccharide, and CO-polysaccharide;

X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and

Ar is pyridinyl or phenyl; and Z is O or S.

In some embodiments, A is N or CR²; B is N or CR⁴; E is N, N⁺X⁴ or CX⁴; G is N, N⁺X³ or CX³; J is N, N⁺X² or CX²; K is N, N⁺X¹ or CX¹; a dashed line is a single or double bond,

R¹, R², R³, R⁴, X¹, X², X³, X⁴, X⁵, Y¹, Y², Y³, and Y⁴ are independently selected from the group consisting of H, deuterium, optionally substituted C₁-C₁₀ alkyl, optionally substituted C₁-C₁₀ deuterated alkyl, optionally substituted C₁-C₁₀ alkenyl, optionally substituted C₁-C₁₀ thioalkyl, optionally substituted C₁-C₁₀ alkoxy, optionally substituted cycloalkyl, optionally substituted heterocycloalkyl, optionally substituted heteroalkyl, optionally substituted aryl, optionally substituted heteroaryl, optionally substituted amido, optionally substituted sulfonyl, optionally substituted amino, optionally substituted sulfonamido, optionally substituted sulfoxyl, cyano, nitro, halogen, hydroxyl, SO₂H₂, optionally substituted C₁-C₁₀ alkoxyalkyl, optionally substituted C₁-C₁₀ carboxy, optionally substituted C₁-C₁₀ alkoxycarbonyl, CO-uronide, CO-monosaccharide, CO-oligosaccharide, and CO-polysaccharide;

X⁶ and X⁷ are independently selected from the group consisting of hydrogen, optionally substituted aryl, optionally substituted heteroaryl, optionally substituted cycloalkyl, optionally substituted heterocycloalkyl, optionally substituted alkylenylaryl, optionally substituted alkylenylheteroaryl, optionally substituted alkylenylheterocycloalkyl, optionally substituted alkylenylcycloalkyl, or X⁶ and X⁷ together form an optionally substituted 5 or 6 membered heterocyclic ring; and

Ar is optionally substituted pyridinyl or optionally substituted phenyl; and Z is O or S.

In some embodiments of any of the preceding methods, said therapeutic is pirfenidone or a pharmaceutically acceptable salt, ester, solvate, or prodrug thereof.

In various embodiments of any of the preceding methods, the therapeutic administered to said patient comprises a compound of formula (II)

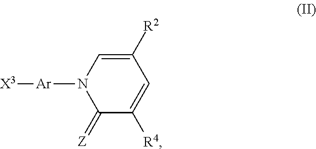

wherein

X³ is H, OH, or C₁₋₁₀alkoxy, Z is O, and R² is methyl, C(═O)H, C(═O(O)CH₃, C(═O)O— glucosyl, fluoromethyl, difluoromethyl, trifluoromethyl, methylmethoxyl, methylhydroxyl, or phenyl; and R⁴ is H or hydroxyl,

or a salt, ester, solvate, or prodrug thereof

In still further embodiments of any of the preceding methods, the therapeutic administered to said patient is selected from the group consisting of

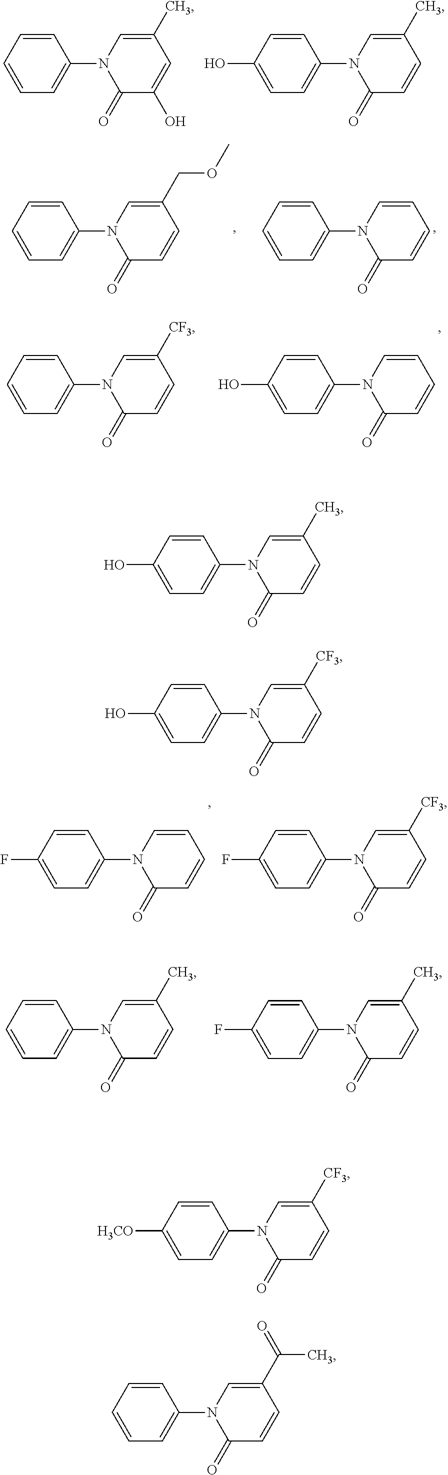

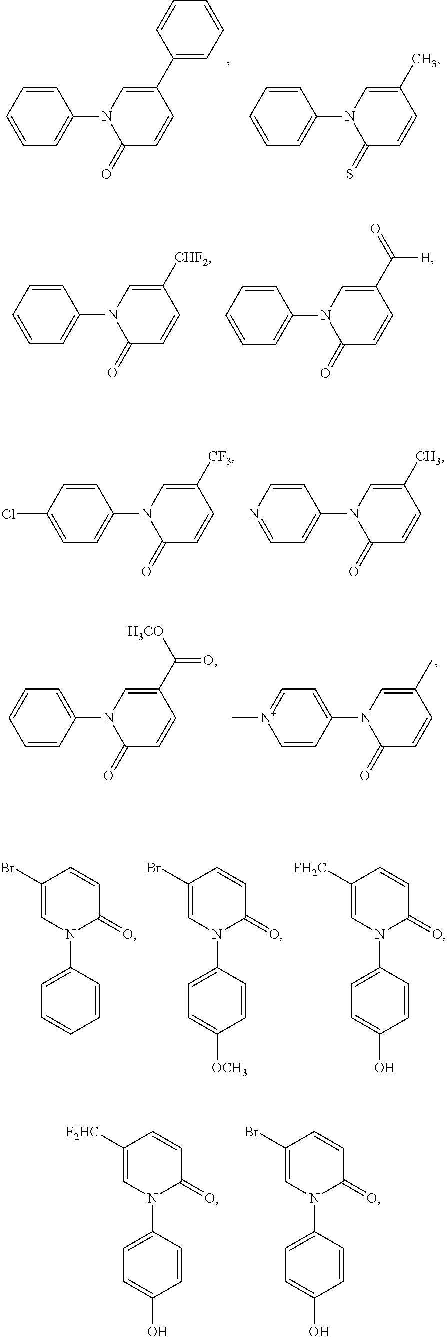

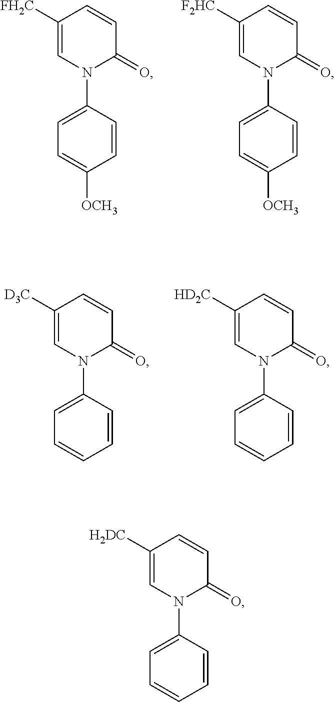

a compound as listed in Table 1,
and pharmaceutically acceptable salts, esters, solvates, and prodrugs thereof.

In some embodiments of any of the preceding methods, the patient is human.

### BRIEF DESCRIPTION OF THE DRAWINGS

FIG. 1 shows that the pirfenidone group (dotted line) had significantly less decline in its ejection fraction, decreasing by only 8% from week 1 to week 5. The ejection fraction for controls decreased by 24% (solid line). The pirfenidone group had a higher ejection fraction of 45% at 5 weeks compared to controls with a mean ejection fraction of 36%, despite the fact that the pirfenidone-treated rats had originally been randomized to a lower ejection fraction at 1 week (54% versus 60%).

FIG. 2 depicts the conduction velocities for the normal, border, and infarct zones of both groups at various pacing cycle lengths, with pirfenidone in the circles and controls as squares. Conduction velocities in the non-infarct zones of both control and pirfenidone groups were fastest among all three zones and were similar between the two groups. Conduction velocities in the infarct zones of both control and pirfenidone groups were slowest among all three zones and were similar between the two groups. Finally, conduction velocities in the border zones of both groups were in between those of the non-infarct and infarct zones. However, the conduction velocities in the border zone for the pirfenidone-treated group was significantly faster, at all pacing cycle lengths, compared to those in the border zone of control animals.

FIG. 3 shows a trend toward lower conduction heterogeneity for pirfenidone-treated rats (circles), compared to control rats (squares).

FIG. 4 shows that, in terms of other electrophysiological parameters, the rise time correlates with conduction velocity. An infarct is shown here to increase the time it takes to fully depolarize for both control (squares) and pirfenidone-treated (circles) rats, with the rise time being slower in the infarct zones compared to their respective normal areas. The rise times in the border zones are in between the infarct and normal zones. The rise time is shown to be shorter for the border zones of pirfenidone-treated rats, consistent with the faster conduction velocities in pirfenidone-treated rats.

FIG. 5 depicts fluorescence amplitude for the three zones. Normal areas had the highest amplitude, infarct areas the least, and border areas in the middle. There was a trend toward higher amplitudes of fluorescence in the border zones of pirfenidone-treated rats, as compared to those of the controls.

FIG. 6 depicts the myocardial infarct size and amount of myocardial fibrosis in control versus pirfenidone-treated rats.

FIG. 7 shows the largest measured frequency gradient over the distance that the gradient occurs for each mapped surface. The dark solid bars represent Control, hatched bars—congestive heart failure (CHF), and open bars—pirfenidone (PFD).

FIG. 8 shows summary correlation coefficient (XC) data for VF activation patterns. Panel A—average XC values for each mapped surface for each group. The dark solid bars represent Control, hatched bars—CHF, and open bars—PFD. Panel B—average XC values for each VF activation patterns for all groups. Panel C—coefficient of variance of the XC values for each VF activation patterns for all groups.

### DETAILED DESCRIPTION OF THE INVENTION

Pirfenidone (PFD) is an orally active, anti-fibrotic agent. It is demonstrated herein that pirfenidone exhibits specific and potent attenuation of post-MI fibrosis, and ameliorates the arrhythmogenic potential of cardiac remodeling.

Pirfenidone is a small drug molecule whose chemical name is 5-methyl-1-phenyl-2-(1H)-pyridone. It is a non-peptide synthetic molecule with a molecular weight of 185.23 daltons. Its chemical elements are expressed as C₁₂H₁₁NO, and its structure and synthesis are known. Several pirfenidone Investigational New Drug Applications (INDs) are currently on file with the U.S. Food and Drug Administration. Human investigations are ongoing or have recently been completed for pulmonary fibrosis, renal glomerulosclerosis, and liver cirrhosis. There have been other Phase II studies that used pirfenidone to attempt to treat benign prostate hypertrophy, hypertrophic scarring (keloids), and rheumatoid arthritis.

Pirfenidone is being investigated for therapeutic benefits to patients suffering from fibrosis conditions such as Hermansky-Pudlak Syndrome (HPS), associated pulmonary fibrosis and idiopathic pulmonary fibrosis (IPF). Pirfenidone is also being investigated for a pharmacologic ability to prevent or remove excessive scar tissue found in fibrosis associated with injured tissues including that of lungs, skin, joints, kidneys, prostate glands, and livers.

Pirfenidone has been reported to inhibit excessive biosynthesis or release of various cytokines such as TNF-α, TGF-β1, bFGF, PDGF, and EGF (Zhang S et al., Australian and New England J Ophthalmology 26:S74-S76 (1998); Cain et al., Int'l J Immunopharmacology 20:685-695 (1998)). Pirfenidone has also been reported to decrease collagen expression and to alter the balance of matrix metalloproteinases (MMPs) and their endogenous inhibitors (tissue inhibitor of metalloproteinases or TIMPs).

### Acute Myocardial Infarction (AMI)

In some embodiments, methods are provided for treating a patient who has suffered an acute myocardial infarction (AMI) comprising administering to the patient a therapeutically effective dose of a therapeutic having an anti-fibrotic effect. In some embodiments, a method is provided for treating a condition caused by ventricular remodeling, wherein the ventricular remodeling is caused by an AMI. In some embodiments, the ventricular remodeling is fibrosis. Thus, in some embodiments a method is provided for reducing ventricular remodeling (e.g., ventricular fibrosis) in a patient who has suffered an AMI. The ventricular remodeling (e.g., ventricular fibrosis) is reduced relative to an amount of ventricular remodeling (e.g., an amount of ventricular fibrosis) in the absence of administration of the therapeutic (e.g., in comparison to a patient who was not administered the therapeutic).

Acute myocardial infarction (AMI) refers to infarction (damage or death) of heart tissue due to an acute, immediate blockage of one or more of the coronary arteries. Coronary arterial occlusion (blockage) due to thrombosis is the cause of most cases of AMI. This blockage restricts the blood supply to the muscle walls of the heart and is often accompanied by symptoms such as chest pain, heavy pressure in the chest, nausea, and shortness of breath, or shooting pain in the left arm. In an acute MI, severe restriction of blood flow in the coronary conduit vessels leads to reduced oxygen delivery to the myocardium and a subsequent cascade of inflammatory reactions resulting in death (infarction) of myocardial tissue. Rapid restoration of blood flow to jeopardized myocardium can limit necrosis and reduce mortality. AMI leads to rapid death of myocytes and vascular structures in the supplied region of the ventricle. The loss of myocytes, arterioles, and capillaries in the infarcted area is irreversible, resulting with time in the formation of scarred tissue.

After the initial cell death due to lack of oxygen, there is a later phase of myocardial cell injury that likely results from an ensuing acute inflammatory reaction (Entman M. L. et al., 1991, FASEB J 5: 2529). Initially, the importance of an inflammatory reaction in mediating myocardial cell injury during AMI was recognized in animal studies which showed that corticosteroids could reduce infarction size by 20 to 35% (Libby P. et al., 1973, J Clin Invest 52: 599; Maclean D. et al., 1978, J Clin Invest 61: 541). However, clinical application of methyl-prednisolone in AMI to minimize myocardial necrosis, was not successful mainly because this treatment interfered with scar formation and healing, leading in some patients to the development of aneurysm and rupture of the ventricle wall (Roberts R. et al., 1976, Circulation 53 Suppl. I: 204). A similar effect has been observed in long-term experiments in rats (Maclean D. et al., 1978, J Clin Invest 61: 541). These disappointing results discouraged further clinical studies that aimed at reducing infarction size by attenuating the inflammatory reaction following AMI.

Patients with AMI can be diagnosed by characteristically elevated levels of troponin, creatine kinase and myoglobin. Troponin levels are now considered the criterion standard in defining and diagnosing MI, according to the American College of Cardiology (ACC)/American Heart Association (AHA) consensus statement on MI. Cardiac troponin levels (troponin-T and troponin-I) have a greater sensitivity and specificity than myocardial muscle creatine kinase (CK-MB) levels in detecting MI. They have important diagnostic and prognostic roles. Positive troponin levels are considered diagnostic of MI in the most recent ACC/AHA revisions, because of their combined specificity and sensitivity in this diagnosis. Serum levels typically increase within 3-12 hours from the onset of chest pain, peak at 24-48 hours, and return to baseline over 5-14 days.

Creatine kinase comprises 3 isoenzymes, including creatine kinase with muscle subunits (CK-MM), which is found mainly in skeletal muscle; creatine kinase with brain subunits (CK-BB), predominantly found in the brain; and myocardial muscle creatine kinase (CK-MB), which is found mainly in the heart. Serial measurements of CK-MB isoenzyme levels were previously the standard criterion for diagnosis of MI. CK-MB levels typically increase within 3-12 hours of onset of chest pain, reach peak values within 24 hours, and return to baseline after 48-72 hours. Levels peak earlier (wash out) if reperfusion occurs. Sensitivity is approximately 95%, with high specificity. However, sensitivity and specificity are not as high as for troponin levels.

Urine myoglobin levels rise within 1-4 hours from the onset of chest pain in AMI. Myoglobin levels are highly sensitive but not specific, and they may be useful within the context of other studies and in early detection of MI in the ED.

The electrocardiogram (ECG) can be an important tool in the initial evaluation and triage of patients in whom an MI is suspected. It is confuniatory of the diagnosis in approximately 80% of cases. It is recommended to obtain an ECG immediately if MI is considered or suspected. In patients with inferior MI, a right-sided ECG is recorded to rule out right ventricular (RV) infarct. Convex ST-segment elevation with upright or inverted T waves is generally indicative of MI in the appropriate clinical setting. ST depression and T-wave changes may also indicate evolution of MI (non-ST-elevated MI). Progression of MI can be evaluated by performing ECGs serially, e.g. daily serial ECGs for the first 2-3 days and additionally as needed.

Imaging studies can be helpful for diagnosis of MI, particularly if the diagnosis is questionable. An echocardiogram can identify regional wall motion abnormalities indicating tissue damage or death. An echocardiogram can also define the extent of the infarction and assess overall left ventricle (LV) and right ventricle (RV) function. In addition, an echocardiogram can identify complications, such as acute mitral regurgitation (MR), LV rupture, or pericardial effusion.

Myocardial perfusion imaging (MPI) utilizes an intravenously administered radiopharmaceutical to depict the distribution of blood flow in the myocardium. The radiopharmaceutical distribution in the heart is imaged using a gamma camera. Perfusion abnormalities, or defects, are assessed and quantified as to location, extent and intensity. Myocardial perfusion imaging can identify areas of reduced myocardial blood flow associated with infarct.

Cardiac catheterization defines the patient's coronary anatomy and the extent of the blockage(s) via cardiac angiography.

AMI may be distinguished from chronic myocardial infarction using any appropriate method known in the art. In some embodiments, the presence of myocardial edema involving a disruption of the energy-regulated ionic transport mechanisms across the cell membrane after the MI is indicative of AMI (Willerson et al., 1977, Am J Pathol 87:159-188). The relatively large extracellular matrix of the developed scar allows gadolinium-based contrast media to accumulate, resulting in DE. T2-weighted CMR sensitively detects infarct-associated myocardial edema (Wisenberg et al., 1988, Am Heart J. 115:510-518; Higgins et al., 1983, Am J Cardiol 52:184-188; Garcia-Dorado et al., 1993, Cardiovasc Res 27:1462-1469) and may be used to differentiate acute from chronic MI. In certain embodiments, a combination of delayed enhancement (DE) and T2-weighted cardiovascular magnetic resonance (CMR) is used to differentiate acute from chronic MI (Abdel-Aty et al., 2004, Circulation 109: 2411-2416).

### Congestive Heart Failure (CHF)

In some embodiments, methods are provided wherein the incidence of congestive heart failure (CHF) or complications of CHF are reduced when a therapeutic having an anti-fibrotic effect is administered to said patient. The incidence of CHF or complications of CHF are reduced relative to the incidence of CHF or complications of CHF in the absence of administration of the therapeutic (e.g., in comparison to a patient who was not administered the therapeutic). The incidence of CHF may be reduced by at least 10% when a therapeutic having an anti-fibrotic effect is administered to a patient in comparison to a patient who was not administered the therapeutic. In further embodiments, the incidence of CHF may be reduced by at least 15%, or at least 20%, or at least 25%, or at least 30%, or at least 35%, or at least 40%, or at least 50%, or at least 55%, or at least 60%, or at least 65%, or at least 70%, or at least 75%, or at least 80%, or at least 85%, or at least 90%, or at least 95% or more when a therapeutic having an anti-fibrotic effect is administered to a patient in comparison to a patient who was not administered the therapeutic.

The prevalence of congestive heart failure has been growing as the population ages and as cardiologists are more successful at reducing mortality from ischemic heart disease, the most common cause of congestive heart failure. Roughly 4.6 million people in the United States have heart failure with an incidence approaching 10 per 1000 after age 65 years. Hospital discharges for congestive heart failure rose from 377,000 in 1979 to 957,000 in 1997 making congestive heart failure the most common discharge diagnosis in people age 65 and over. The five year mortality from congestive heart failure approaches 50%.

CHF may be a complication of AMI and results from a decline in the pumping capacity of the heart. CHF can also result from cardiac malformations, such as valve disease, or other disorders that damage cardiac tissue, e.g. cardiac myopathy. Due to the activation of one or more compensatory mechanisms, the damaging changes caused by CHF can be present and ongoing even while the patient remains asymptomatic. In fact, the compensatory mechanisms which maintain normal cardiovascular function during the early phases of CHF may actually contribute to progression of the disease, for example by exerting deleterious effects on the heart and circulation.

Some of the more important pathophysiologic changes which occur in CHF are activation of the hypothalamic-pituitary-adrenal axis, systemic endothelial dysfunction and myocardial remodeling.

Therapies specifically directed at counteracting the activation of the hypothalamic-pituitary-adrenal axis include beta-adrenergic blocking agents (β-blockers), angiotensin converting enzyme (ACE) inhibitors, certain calcium channel blockers, nitrates and endothelin-1 blocking agents. Calcium channel blockers and nitrates, while producing clinical improvement have not been clearly shown to prolong survival whereas β-blockers and ACE inhibitors have been shown to significantly prolong life, as have aldosterone antagonists.

Systemic endothelial dysfunction is a well-recognized feature of CHF and is clearly present by the time signs of left ventricular dysfunction are present. Endothelial dysfunction is important with respect to the intimate relationship of the myocardial microcirculation with cardiac myocytes. The evidence suggests that microvascular dysfunction contributes significantly to myocyte dysfunction and the morphological changes which lead to progressive myocardial failure.

Myocardial remodeling is a complex process which accompanies the transition from asymptomatic to symptomatic heart failure, and may be described as a series of adaptive changes within the myocardium. Components of myocardial remodeling may include fibrosis, alterations in myocyte biology, loss of myocytes by necrosis or apoptosis, alterations in the extracellular matrix and alterations in left ventricular chamber geometry.

The diagnosis of congestive heart failure is most often a clinical one that is based on knowledge of the patient's pertinent medical history, a careful physical examination, and selected laboratory tests. Symptoms include dyspnea (shortness of breath) which worsens upon lying supine, fluid retention and swelling in the lungs and extremities, e.g. with pulmonary rales or edema in the legs.

Congestive heart failure is strongly suggested by the presence of cardiomegaly (enlarged heart) or pulmonary vascular congestion on chest X-ray. Electrocardiogram (ECG) may show anterior Q waves or left bundle branch block on the electrocardiogram. The echocardiogram is the diagnostic standard for identifying congestive heart failure. The patient may undergo two-dimensional echocardiography with Doppler flow studies. Radionuclide angiography or contrast cineangiography may be helpful if the echocardiogram is equivocal.

### Preservation of Viable Cardiac Tissue and Reduction of Infarct Size

In some embodiments, methods are provided wherein the cardiac tissue is preserved from necrosis when a therapeutic having an anti-fibrotic effect is administered to a patient suffering an AMI, in comparison to the amount of viable cardiac tissue in the absence of administration of the therapeutic (e.g., in comparison to a patient who was not administered a therapeutic). The amount of cardiac tissue preserved from necrosis can be increased at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. The increase in viable cardiac tissue can be determined by MRI or computerized tomography (CT) scan.

Methods are also provided herein to control or reduce myocardial infarct size. “Control” or “controlling” as used herein means to reduce, reduce the incidence of, or prevent the progression of a disorder. In some cases, methods are provided wherein the infarct size of a patient is reduced when a therapeutic is administered to said patient, in comparison to the infarct size of a patient in the absence of administration of the therapeutic (e.g., in comparison to a patient who was not administered a therapeutic). The infarct size can be reduced at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. The reduction in infarct size can be determined by MRI and/or by voltage/conduction mapping.

In some embodiments, methods are provided wherein the cardiac function is preserved when a therapeutic having an anti-fibrotic effect is administered to a patient suffering an AMI, in comparison to the cardiac function of a patient suffering an AMI in the absence of administration of the therapeutic (e.g., in comparison to a patient who was not administered a therapeutic). Preservation of cardiac function can be determined by measuring ejection fraction using echocardiography, wherein the ejection fraction can be improved by at least 1%, at least 3%, at least 5%, at least 7%, at least 10%, at least 12%, or at least 15%. Preservation of cardiac tissue can also be determined by measuring ejection fraction using MRI, wherein the ejection fraction can be improved by at least 1%, at least 3%, at least 5%, at least 7%, at least 10%, at least 12%, or at least 15%, and/or the infarct size can be decreased by at least 1%, at least 3%, at least 5%, at least 7%, at least 10%, at least 12% or at least 15%. Other methods of determining cardiac function are known in the art and include but are not limited to nuclear imaging, functional capacity, exercise capacity, New York Heart Association (NYHA) functional classification system, and myocardial oxygen consumption (MVO2).

### Reduction in the Incidence of Ventricular Tachycardia

In other cases, methods are provided wherein the incidence of ventricular tachycardia in a patient is reduced when a therapeutic is administered to said patient, in comparison to the incidence of ventricular tachycardia in a patient who was not administered the therapeutic. The incidence of ventricular tachycardia can be reduced at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. The reduction in incidence of tachycardia can be determined by electrocardiogram (ECG or EKG) or by echocardiogram.

### Ventricular Fibrillation

In some embodiments, methods are provided for treating or preventing ventricular fibrillation in a patient in need thereof, comprising administering to the patient a therapeutic having an anti-fibrotic effect. In some embodiments, the amount or degree of ventricular fibrillation is reduced relative to the amount or degree of ventricular fibrillation in the absence of administration of the therapeutic.

Ventricular fibrillation (VF) is a condition in which the heart's electrical activity becomes disordered. When this happens, the heart's ventricles contract in a rapid, unsynchronized way. The ventricles “quiver” rather than beat, causing the heart to pump little or no blood.

VF is life threatening and requires prompt treatment. Without medical treatment, collapse and sudden cardiac death can occur. Ventricular fibrillation (VF) may occur spontaneously with unpredictable timing and requires specialized tests to acquire an accurate diagnosis.

VF may be diagnosed using an electrocardiogram (ECG or EKG), e.g. a Holter Monitor—A Holter monitor is a small, portable machine that records the patient's ECG and is typically worn for 24 hours. This monitor may detect arrhythmias that might not show up on a resting electrocardiogram, which only records a heartbeat for a few seconds at rest.

VF may also be diagnosed using an event monitor—This is a small monitor about the size of a pager that the patient can have for up to a month. Since the arrhythmia may occur at unpredictable times, this monitor records the abnormal rhythm when the patient signals that he or she is experiencing symptoms.

An exercise stress or treadmill test also may be used to diagnose VF, by recording the electrical activity of the patient's heart during exercise, which differs from the heart's electrical activity at rest.

Another method of diagnosing VF is through an electrophysiology study. In an electrophysiology (EP) study, physiciansinsert special electrode catheters—long, flexible wires—into veins and guide them into the heart. These catheters sense electrical impulses and also may be used to stimulate different areas of the heart. Physicians can then locate the sites that are causing arrhythmias. The EP study allows physicians to examine an arrhythmia under controlled conditions and acquire more accurate, detailed information than with any other diagnostic test.

VF can be monitored and measured by any one or more of the parameters described, for example, in Example 5 below. In some embodiments, the incidence of VF can be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, compared to incidence of VF in a patient who was not administered the therapeutic.

### Sudden Cardiac Death

Sudden cardiac death (also called sudden arrest) is death resulting from an abrupt loss of heart function (cardiac arrest). The victim may or may not have diagnosed heart disease. The time and mode of death are unexpected. It occurs within minutes after symptoms appear. The most common underlying reason for patients to die suddenly from cardiac arrest is AMI due to coronary heart disease. Other types of arrhythmia can also cause cardiac arrest.

Most of the cardiac arrests that lead to sudden death occur when the electrical impulses in the diseased heart become rapid (ventricular tachycardia) or chaotic (ventricular fibrillation) or both. This irregular heart rhythm (arrhythmia) causes the heart to suddenly stop beating. Some cardiac arrests are due to extreme slowing of the heart, bradycardia. If a cardiac arrest was due to ventricular tachycardia or ventricular fibrillation, survivors are at higher risk for another arrest, especially if they have underlying heart disease.

Therefore, in some cases, methods are provided wherein the incidence of sudden cardiac death is reduced when a therapeutic having an anti-fibrotic effect is administered to said patient, in comparison to the incidence of cardiac death in a patient who was not administered a therapeutic. The incidence of sudden cardiac death can be reduced at least 5° A, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

### Arrhythmia

Methods of the invention are contemplated to control arrhythmia by administering a therapeutic having an anti-fibrotic effect. In some embodiments, a method is provided to reduce the incidence or risk of arrhythmia. The incidence or risk can be reduced at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

An arrhythmia is an abnormal heart rhythm. In an arrhythmia the heartbeats may be too slow, too rapid, too irregular, or too early. There are many types of arrhythmias, including premature atrial contractions (early extra beats that originate in the atria (upper chambers of the heart), premature ventricular contractions (PVCs) (skipped heartbeat), atrial fibrillation (an irregular heart rhythm that causes the atria, the upper chambers of the heart to contract abnormally), atrial flutter (an arrhythmia caused by one or more rapid circuits in the atrium), paroxysmal supraventricular tachycardia (PSVT) (a rapid heart rate, usually with a regular rhythm, originating from above the ventricles), accessory pathway tachycardias (a rapid heart rate due to an extra abnormal pathway or connection between the atria and the ventricles), AV nodal reentrant tachycardia (a rapid heart rate due to more than one pathway through the AV node), ventricular tachycardia (VT) (a rapid heart rhythm originating from the lower chambers (or ventricles) of the heart), ventricular fibrillation (an erratic, disorganized firing of impulses from the ventricles), bradyarrhythmias (slow heart rhythms, which may arise from disease in the heart's electrical conduction system), and/or long QT syndrome (the QT interval is the area on the electrocardiogram (ECG) that represents the time it takes for the heart muscle to contract and then recover, or for the electrical impulse to fire impulses and then recharge). When the QT interval is longer than normal, it increases the risk for “torsade de pointes,” a life-threatening form of ventricular tachycardia.

Symptoms of arrhythmia include chest pain, fainting, fast or slow heartbeat (palpitations), light-headedness, dizziness, paleness, shortness of breath, skipping beats, changes in the pattern of the pulse, and sweating. Arrythmias may be diagnosed by those of skill in the art using such methods as electrocardiogram, Holter monitor, event monitor, stress test, echocardiogram, cardiac catheterization, electrophysiology study (EPS), and head-up tilt table test.

The amount of a therapeutic effective to control arrhythmia may be an amount effective to reduce ventricular remodeling, e.g. in an animal model or during clinical trial. Ventricular remodeling refers to the changes in size, shape, and function of the heart after injury to the left ventricle. The injury is typically due to AMI. In some embodiments, the ventricular remodeling is due to ventricular fibrosis caused by an AMI. The remodeling process is characterized by progressive expansion of the initial infarct area and dilation of the left ventricular lumen, with cardiomyocyte replacement by fibrous tissue deposition in the ventricular wall (Kocher et al., 2001, Nature Medicine 7(4): 430-6). Another integral component of the remodeling process is the development of neoangiogenesis within the myocardial infarct scar, a process requiring activation of latent collagenase and other proteinases. Under normal circumstances, the contribution of neoangiogenesis to the infarct-bed capillary network is insufficient to keep pace with the tissue growth required for contractile compensation and is unable to support the greater demands of the hypertrophied but viable myocardium. The relative lack of oxygen and nutrients to the hypertrophied myocytes might be an important etiological factor in the death of otherwise viable myocardium, resulting in progressive infarct extension and fibrous replacement. Late reperfusion of the infarct vascular bed in both humans and animal models is known to significantly benefit ventricular remodeling and survival (Kocher et al., 2001, Nature Medicine 7(4): 430-6).

### Therapeutic Agents

Therapeutic agents used in the disclosed methods can be any therapeutic agent that affects fibrosis. Contemplated agents include agents that reduce the activity of transforming growth factor-beta (TGF-β) (including but not limited to GC-1008 (Genzyme/MedImmune); lerdelimumab (CAT-152; Trabio, Cambridge Antibody); metelimumab (CAT-192, Cambridge Antibody,); LY-2157299 (Eli Lilly); ACU-HTR-028 (Opko Health)) including antibodies that target one or more TGF-β isoforms, inhibitors of TGF-β receptor kinases TGFBR1 (ALK5) and TGFBR2, and modulators of post-receptor signaling pathways; chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; zibotentan); agents that reduce the activity of connective tissue growth factor (CTGF) (including but not limited to FG-3019, FibroGen), and also including other CTGF-neutralizing antibodies; matrix metalloproteinase (MMP) inhibitors (including but not limited to MMPI-12, PUP-1 and tigapotide triflutate); agents that reduce the activity of epidermal growth factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Rα) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573); agents that reduce levels of copper such as tetrathiomolybdate; agents that reduce oxidative stress including N-acetyl cysteine and tetrathiomolybdate; and interferon gamma. Also contemplated are agents that are inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil. UK-369003, vardenafil, and zaprinast); or modifiers of the arachidonic acid pathway including cyclooxygenase and 5-lipoxegenase inhibitors (including but not limited to Zileuton). Further contemplated are compounds that reduce tissue remodeling or fibrosis including prolyl hydrolase inhibitors (including but not limited to 1016548, CG-0089, FG-2216, FG-4497, FG-5615, FG-6513, tibrostatin A (Takeda), lufironil, P-1894B, and safironil) and peroxisome proliferator-activated receptor (PPAR)-gamma agonists. (including but not limited to pioglitazone and rosiglitazone,)

In some embodiments, formula (I), (II), (III), V or (V) defined above are

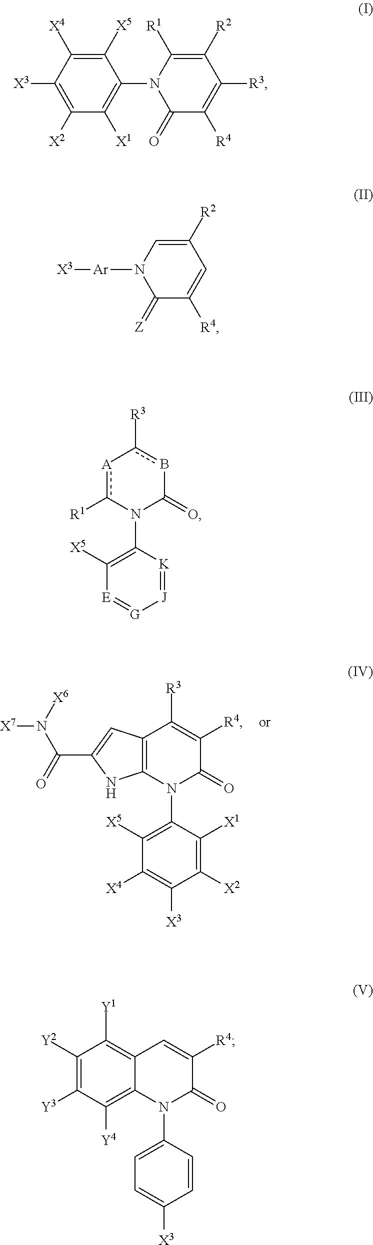

wherein

R¹, R², R³, R⁴, X¹, X², X³, X⁴, X⁵, Y¹, Y², Y³, and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, C₁-C₁₀ carboxy, alkoxycarbonyl, CO-uronide, CO-monosaccharide, CO-oligosaccharide, and CO-polysaccharide;

X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and

Ar is pyridinyl or phenyl; and Z is O or S;

or a pharmaceutically acceptable salt, ester, solvate, or prodrug of pirfenidone or the compound of formula (I), (II), (III), (IV), or (V).

In some embodiments, R¹, R², R³, R⁴, X¹, X², X³, X⁴, X⁵, Y¹, Y², Y³, and Y⁴ are independently optionally substituted pyrazinyl, optionally substituted pyridazinyl, optionally substituted pyrrolyl, optionally substituted thiophenyl, optionally substituted thiazolyl, optionally substituted oxazolyl, optionally substituted imidazolyl, optionally substituted isoxazolyl, optionally substituted pyrazolyl, optionally substituted isothiazolyl, optionally substituted napthyl, optionally substituted quinolinyl, optionally substituted isoquinolinyl, optionally substituted quinoxalinyl, optionally substituted benzothiazolyl, optionally substituted benzothiophenyl, optionally substituted benzofuranyl, optionally substituted indolyl, or optionally substituted benzimidazolyl,

In some cases, the therapeutic is a compound of formula (II), wherein X³ is H, OH, or C₁₋₁₀alkoxy, Z is O, and R² is methyl, C(═O)H, C(═O)CH₃, C(═O)O-glucosyl, fluoromethyl, difluoromethyl, trifluoromethyl, methylmethoxyl, methylhydroxyl, or phenyl; and R⁴ is H or hydroxyl.

Some specific contemplated compounds of formula (II) include

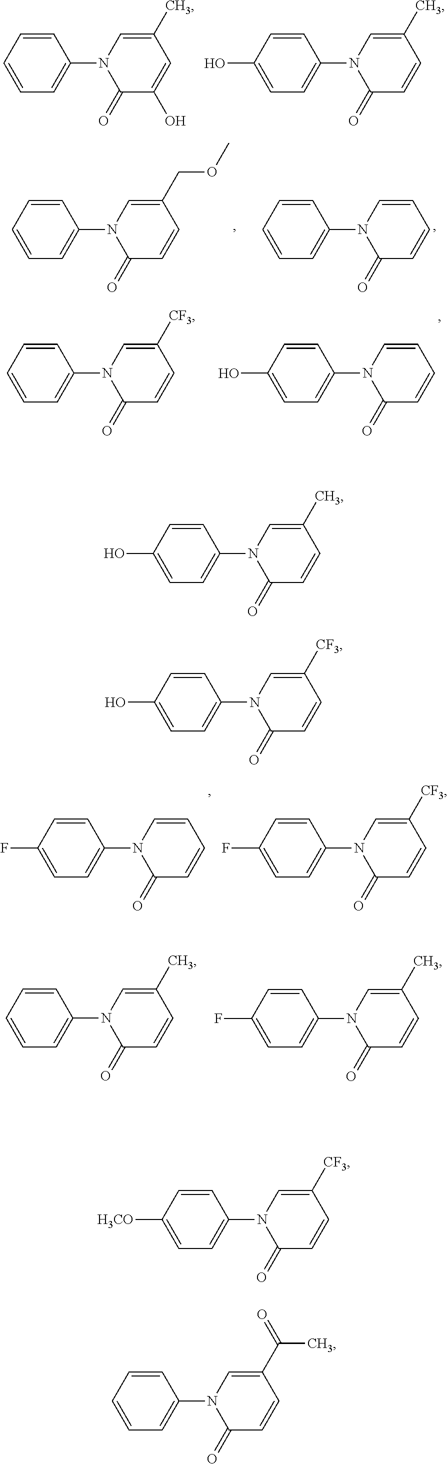

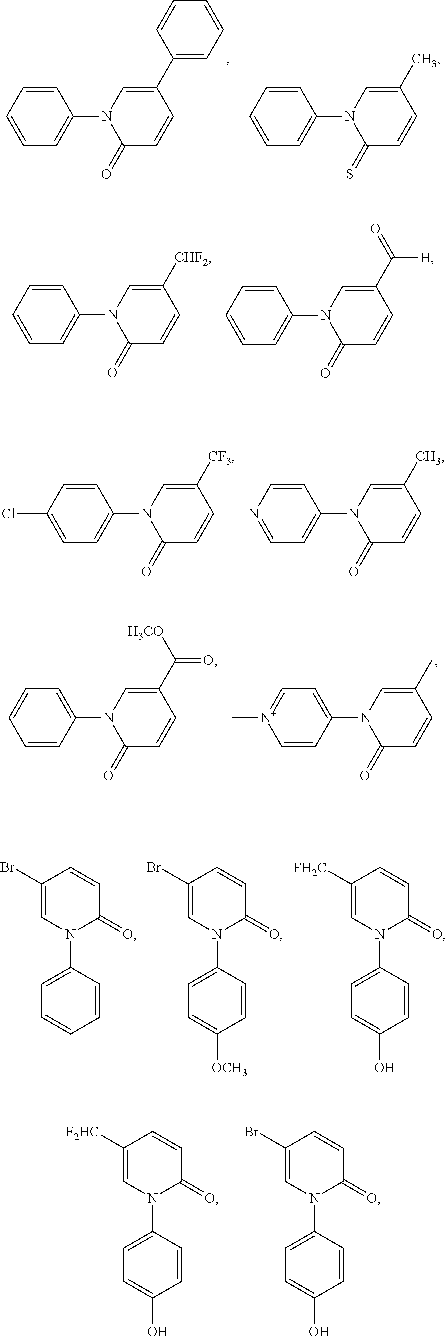

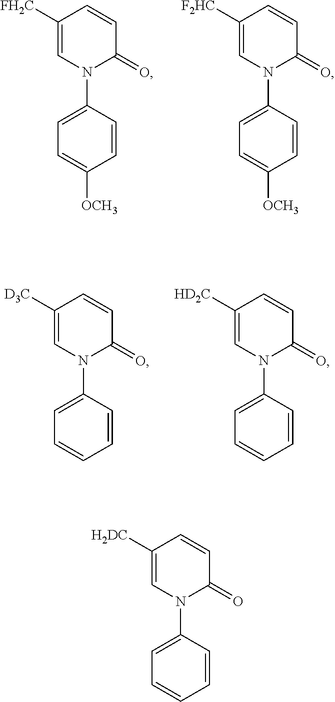

, a compound listed in Table 1, below, and pharmaceutically acceptable salts, esters, solvates, and prodrugs thereof.

Other specific therapeutic agents contemplated include relaxin, ufironil, surifonil, a TGF-β antibody, CAT-192, CAT-158; ambresentan, thelin; FG-3019, a CTGF antibody; anti-EGFR antibody; a EGFR kinase inhibitor; tarceva; gefitinib; PDGF antibody, PDGFR kinase inhibitor; gleevec; BIBF-1120, VEGF, FGF, and PDGF receptor inhibitor; anti-integrin antibody; IL-4 antibody; tetrathiomolybdate, a copper chelating agent; interferon-gamma; NAC, a cysteine pro-drug; hepatocyte growth factor (HGF); KGF; angiotension receptor blockers, ACE inhibitors, rennin inhibitors; COX and LO inhibitors; Zileuton; monteleukast; avastin; statins; PDE5 inhibitors, such as sildenafil, udenafil, tadalafil, vardenafil, or zaprinast; rofumilast; etanercept (Enbrel); procoagulant; prostaglandins, such as PGE2, PRX-08066, a 5HT_{2B} receptor antagonist; cintredekin besudotox, a chimeric human IL13 conjugated to a genetically engineered Pseudomonas exotoxin; roflumilast, a PDE4 inhibitor; FG-3019, an anti-connective tissue growth factor human monoclonal antibody; GC-1008, a TGF-β human monoclonal antibody; treprostinil, a prostacyclin analog; interferon-α; QAX-576, a IL13 modulator; WEB 2086, a PAF-receptor antagonist; imatinib mesylate; FG-1019; Suramin; Bosentan; IFN-1b; anti-IL-4; anti-IL-13; taurine, niacin, NF-κB antisense oligonucleotides; and nitric oxide synthase inhibitors. Also contemplated are peroxisome proliferator-activated receptor (PPAR)-gamma agonists, including but not limited to pioglitazone and rosiglitazone

The term “alkyl” used herein refers to a saturated or unsaturated straight or branched chain hydrocarbon group of one to ten carbon atoms, including, but not limited to, methyl, ethyl, n-propyl, isopropyl, n-butyl, isobutyl, tert-butyl, n-hexyl, and the like. Alkyls of one to six carbon atoms are also contemplated. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, cycloalkyl, heterocycloalkyl, and amino. It is specifically contemplated that in the analogs described herein the alkyl group consists of 1-40 carbon atoms, preferably 1-25 carbon atoms, preferably 1-15 carbon atoms, preferably 1-12 carbon atoms, preferably 1-10 carbon atoms, preferably 1-8 carbon atoms, and preferably 1-6 carbon atoms. “Heteroalkyl” is defined similarly as alkyl, except the heteroalkyl contains at least one heteroatom independently selected from the group consisting of oxygen, nitrogen, and sulfur.

As used herein, the term “cycloalkyl” refers to acyclic hydrocarbon group, e.g., cyclopropyl, cyclobutyl, cyclohexyl, and cyclopentyl. “Heterocycloalkyl” is defined similarly as cycloalkyl, except the ring contains one to three heteroatoms independently selected from the group consisting of oxygen, nitrogen, and sulfur. Nonlimiting examples of heterocycloalkyl groups include piperidine, tetrahydrofuran, tetrahydropyran, dihydrofuran, morpholine, thiophene, and the like. Cycloalkyl and heterocycloalkyl groups can be saturated or partially unsaturated ring systems optionally substituted with, for example, one to three groups, independently selected from the group consisting of alkyl, alkyleneOH, C(O)NH₂, NH₂, oxo (═O), aryl, haloalkyl, halo, and OH. Heterocycloalkyl groups optionally can be further N-substituted with alkyl, hydroxyalkyl, alkylenearyl, or alkyleneheteroaryl.

The term “alkenyl” used herein refers to a straight or branched chain hydrocarbon group of two to ten carbon atoms containing at least one carbon double bond including, but not limited to, 1-propenyl, 2-propenyl, 2-methyl-1-propenyl, 1-butenyl, 2-butenyl, and the like.

The term “halo” used herein refers to fluoro, chloro, bromo, or iodo.

The term “alkylene” used herein refers to an alkyl group having a substituent. For example, the term “alkylene aryl” refers to an alkyl group substituted with an aryl group. The alkylene group is optionally substituted with one or more substituent previously listed as an optional alkyl substituent. For example, an alkylene group can be —CH2CH2-.

As used herein, the term “alkenylene” is defined identical as “alkylene,” except the group contains at least one carbon-carbon double bond.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF3, NO2, CN, NC, OH, alkoxy, amino, CO2H, CO2alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

The term “deuterated alkyl” used herein refers to an alkyl group substituted with one or more deuterium atoms (D).

The term “thioalkyl” used herein refers to one or more thio groups appended to an alkyl group.

The term “hydroxyalkyl” used herein refers to one or more hydroxy groups appended to an alkyl group.

The term “alkoxy” used herein refers to straight or branched chain alkyl group covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “alkoxyalkyl” used herein refers to one or more alkoxy groups appended to an alkyl group.

The term “arylalkoxy” used herein refers to a group having an aryl appended to an alkoxy group. A non-limiting example of an arylalkoxy group is a benzyloxy (Ph-CH₂—O—).

The term “amino” as used herein refers to —NR₂, where R is independently hydrogen, optionally substituted alkyl, optionally substituted heteroalkyl, optionally substituted cycloalkyl, optionally substituted heterocycloalkyl, optionally substituted aryl or optionally substituted heteroaryl. Non-limiting examples of amino groups include NH₂ and N(CH₃)₂. In some cases, R is independently hydrogen or alkyl.

The term “amido” as used herein refers to —C(O)NH₂, —C(O)NR₂, —NRC(O)R or —NHC(O)H, where each R is independently hydrogen, optionally substituted alkyl, optionally substituted heteroalkyl, optionally substituted cycloalkyl, optionally substituted heterocycloalkyl, optionally substituted aryl or optionally substituted heteroaryl. In some cases, the amido group is —NHC(O)alkyl or —NHC(O)H. A non-limiting example of an amido group is —NHC(O)CH₃.

The term “carboxy” or “carboxyl” used herein refers to COOH or its deprotonated form —COO⁻. C₁₋₁₀carboxy refers to optionally substituted alkyl or alkenyl groups having a carboxy moiety. Examples include, but are not limited to, —CH₂COOH, —CH₂CH(COOH)CH₃, and —CH₂CH₂CH₂COOH.

The term “alkoxycarbonyl” refers to —(CO)—O-alkyl, wherein the alkyl group can optionally be substituted. Examples of alkoxycarbonyl groups include, but are not limited to, methoxycarbonyl group, ethoxycarbonyl group, propoxycarbonyl group, and the like.

The term “alkylcarbonyl” refers to —(CO)-alkyl, wherein the alkyl group can optionally be substituted. Examples of alkylcarbonyl groups include, but are not limited to, methylcarbonyl group, ethylcarbonyl group, propylcarbonyl group, and the like.

The term “sulfonamido” refers to —SO₂NR₂, wherein R is independently hydrogen, optionally substituted heteroalkyl, optionally substituted cycloalkyl, optionally substituted heterocycloalkyl, optionally substituted aryl or optionally substituted heteroaryl. In some cases, the sulfonamido group is —SO₂NR₂ where R is independently hydrogen or an optionally substituted alkyl. Examples of a sulfonamido group include, but are not limited to, —SO₂N(CH₃)₂ and —SO₂NH₂.

The term “sulfonyl” refers to —SO₂R, where R is independently hydrogen or an optionally substituted alkyl, optionally substituted heteroalkyl, optionally substituted cycloalkyl, optionally substituted heterocycloalkyl, optionally substituted aryl or optionally substituted heteroaryl. In some cases, a sulfonyl group is —SO₂alkyl, wherein the alkyl group can optionally be substituted. One example of a sulfonyl group is methylsulfonyl (e.g., —SO₂CH₃).

The term “sulfoxyl” refers to —SOR, where each R is independently hydrogen or an optionally substituted alkyl, optionally substituted heteroalkyl, optionally substituted cycloalkyl, optionally substituted heterocycloalkyl, optionally substituted aryl or optionally substituted heteroaryl. One example of a sulfonyl group is methylsulfonyl (e.g., —SOCH₃).

Carbohydrates are polyhydroxy aldehydes or ketones, or substances that yield such compounds upon hydrolysis. Carbohydrates comprise the elements carbon (C), hydrogen (H) and oxygen (O) with a ratio of hydrogen twice that of carbon and oxygen. In their basic form, carbohydrates are simple sugars or monosaccharides. These simple sugars can combine with each other to form more complex carbohydrates. The combination of two simple sugars is a disaccharide. Carbohydrates consisting of two to ten simple sugars are called oligosaccharides, and those with a larger number are called polysaccharides.

The term “uronide” refers to a monosaccharide having a carboxyl group on the carbon that is not part of the ring. The uronide name retains the root of the monosaccharide, but the -ose sugar suffix is changed to -uronide. For example, the structure of glucuronide corresponds to glucose.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, a substituted group is derived from the unsubstituted parent structure in which there has been an exchange of one or more hydrogen atoms for another atom or group. A “substituent group,” as used herein, means a group selected from the following moieties:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl, and

(B) alkyl, heteroalkyl, cycloalkyl, heterocycloalkyl, aryl, amino, amido, carbonyl, thiocarbonyl, alkoxycarbonyl, silyl, sulfonyl, sulfoxyl, alkoxy, aryloxy, and heteroaryl, substituted with at least one substituent selected from:
(i) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl, and
(ii) alkyl, heteroalkyl, cycloalkyl, heterocycloalkyl, aryl, amino, amido, carbonyl, thiocarbonyl, alkoxycarbonyl, silyl, sulfonyl, sulfoxyl, alkoxy, aryloxy, and heteroaryl, substituted with at least one substituent selected from:
   (a) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl, and
   (b) alkyl, heteroalkyl, cycloalkyl, heterocycloalkyl, aryl, amino, amido, carbonyl, thiocarbonyl, alkoxycarbonyl, silyl, sulfonyl, sulfoxyl, alkoxy, aryloxy, and heteroaryl, substituted with at least one substituent selected from —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl.

In some embodiments, the substituent group is a “size-limited substituent” or “size-limited substituent group,” which refers to a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

In some embodiments, the substituent group is a “lower substituent” or “lower substituent group,” which refers to a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

In some cases, the substituent group(s) is (are) one or more group(s) individually and independently selected from alkyl, cycloalkyl, aryl, fused aryl, heterocyclyl, heteroaryl, hydroxy, alkoxy, aryloxy, mercapto, alkylthio, arylthio, cyano, halo, carbonyl, thiocarbonyl, alkoxycarbonyl, nitro, silyl, trihalomethanesulfonyl, trifluoromethyl, and amino, including mono and di substituted amino groups, and the protected derivatives thereof

The protecting groups that can form the protective derivatives of the above substituents are known to those of skill in the art and can be found in references such as Greene and Wuts, Protective Groups in Organic Synthesis; 3^{rd} Edition, John Wiley and Sons: New York, 2006. Wherever a substituent is described as “optionally substituted” that substituent can be substituted with the above-described substituents.

Asymmetric carbon atoms can be present. All such isomers, including diastereomers and enantiomers, as well as the mixtures thereof are intended to be included in the scope of the disclosure herein. In certain cases, compounds can exist in tautomeric forms. All tautomeric forms are intended to be included in the scope of the disclosure herein. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated.

Compounds that can be used in the disclosed methods include those described in U.S. Patent Publication No. 2007/0049624 (US national stage of WO 05/0047256), International Publication No. WO 03/068230, WO 08/003,141, WO 08/157,786, or in U.S. Pat. Nos. 5,962,478; 6,300,349; 6,090,822; 6,114,353; Re. 40,155; 6,956,044; or 5,310,562. Synthesis of the compounds used in the disclosed methods can be by any means known in the art, including those described in the patents and patent publications listed herein. Other synthetic means can be used and are within the knowledge of the skilled artisan.

One class of compounds contemplated for use in the disclosed methods is a deuterated (D) form of any of the compounds disclosed herein. One specific such compound is a compound having a CD₃ moiety and/or a D to replace any or all of the methyl or hydrogens of pirfenidone. Examples include

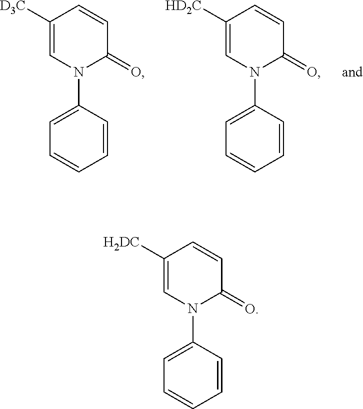

The synthesis of these compounds can be found in International Patent Publication No. WO 08/157,786.

Some specific compounds of formula (I), (II), (III), or (IV) are listed in Table 1. Description of the synthesis of these compounds can be found in U.S. Provisional Application Nos. 61/058,436, filed Jun. 3, 2008 and 61/074,446, filed Jun. 20, 2008, the disclosures of which are each incorporated by reference herein.

TABLE 1

| Cmpd No. | Structure |
|---|---|
| 1 | 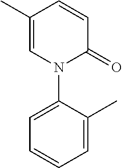 |
| 2 | 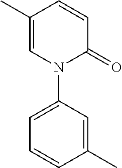 |
| 3 | 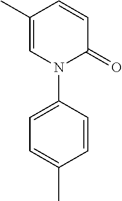 |
| 4 | 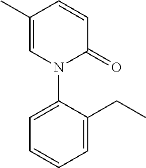 |
| 5 | 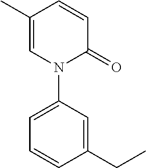 |
| 6 | 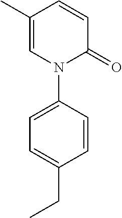 |
| 7 | 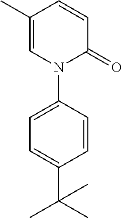 |
| 8 | 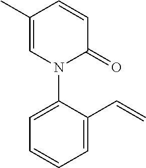 |
| 9 | 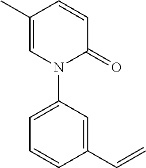 |
| 10 | 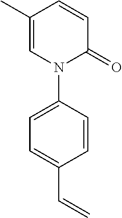 |
| 11 | 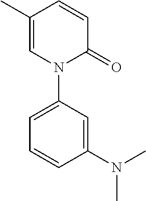 |
| 12 | 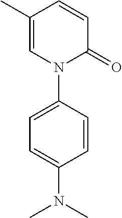 |
| 13 | 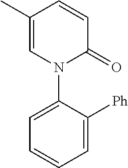 |
| 14 | 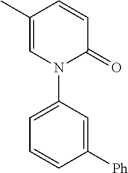 |
| 15 | 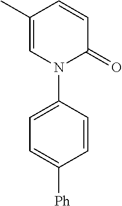 |
| 16 | 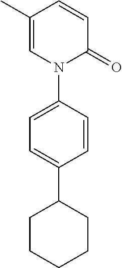 |
| 17 | 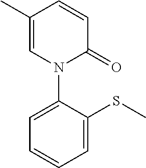 |
| 18 | 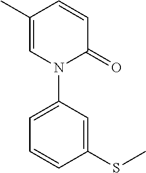 |
| 19 | 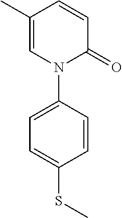 |
| 20 | 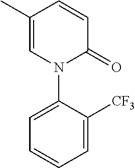 |
| 21 | 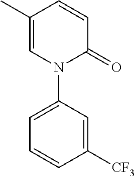 |
| 22 | 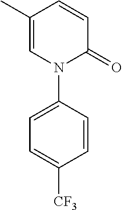 |
| 23 | 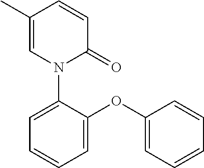 |
| 24 | 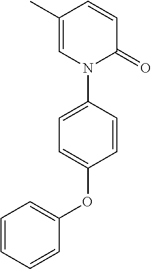 |
| 25 | 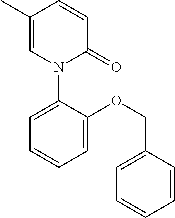 |
| 26 | 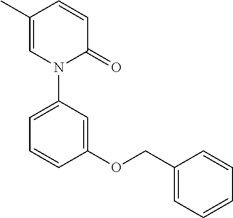 |
| 27 | 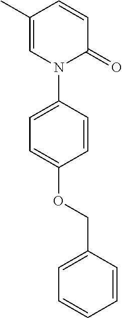 |
| 28 | 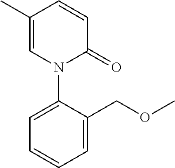 |
| 29 | 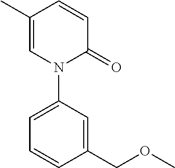 |
| 30 | 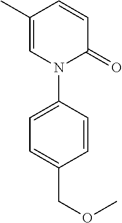 |
| 31 | 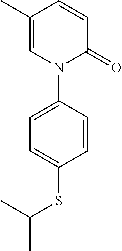 |
| 32 | 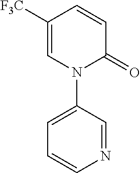 |
| 33 | 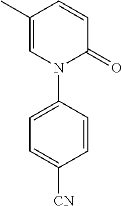 |
| 34 | 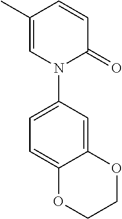 |
| 35 | 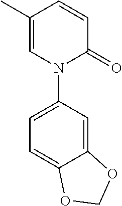 |
| 36 | 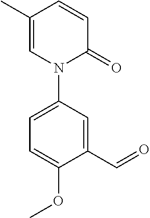 |
| 37 | 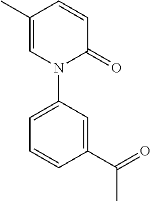 |
| 38 | 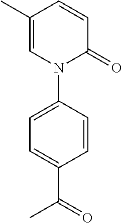 |
| 39 | 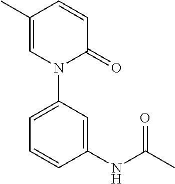 |
| 40 | 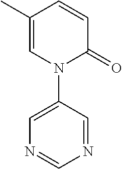 |
| 41 | 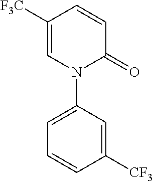 |
| 42 | 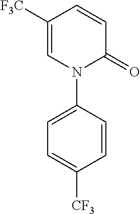 |
| 43 | 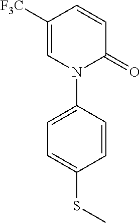 |
| 44 | 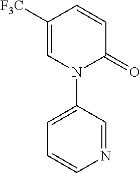 |
| 45 | 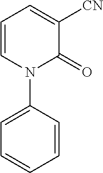 |
| 46 | 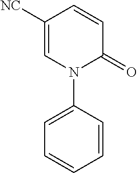 |
| 47 | 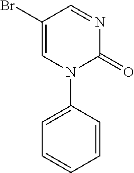 |
| 48 | 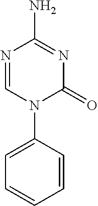 |
| 49 | 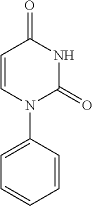 |
| 50 | 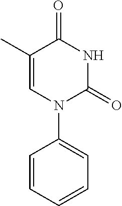 |
| 51 | 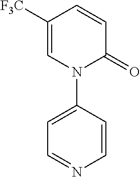 |
| 52 | 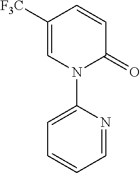 |
| 53 | 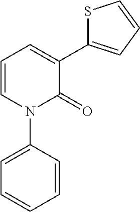 |
| 54 | 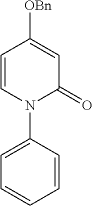 |
| 55 | 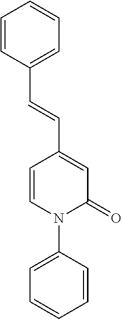 |
| 56 | 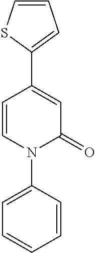 |
| 57 | 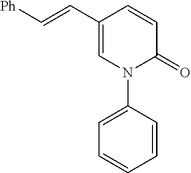 |
| 58 | 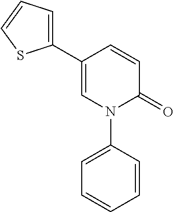 |
| 59 | 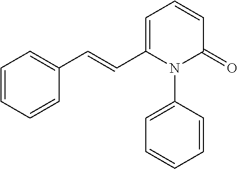 |
| 60 | 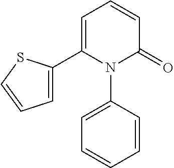 |
| 61 | 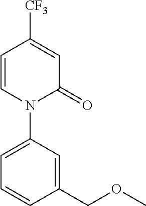 |
| 62 | 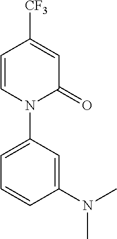 |
| 63 | 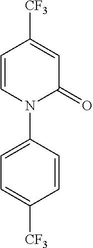 |
| 64 | 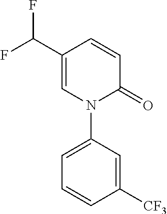 |
| 65 | 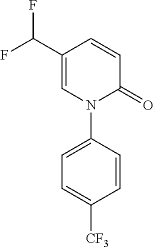 |
| 66 | 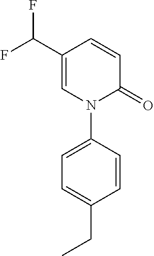 |
| 67 | 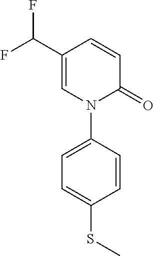 |
| 68 | 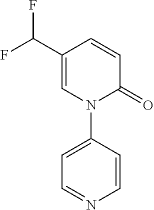 |
| 69 | 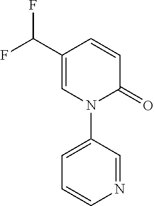 |
| 70 | 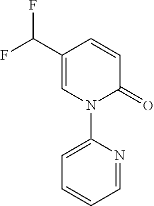 |
| 71 | 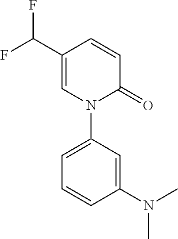 |
| 72 | 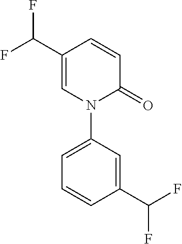 |
| 73 | 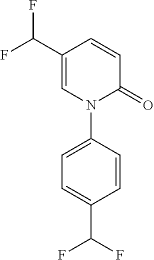 |
| 74 | 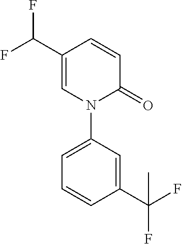 |
| 75 | 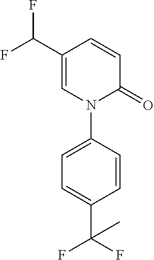 |
| 76 | 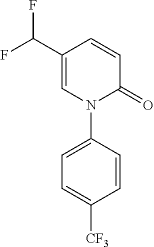 |
| 77 | 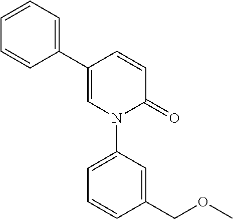 |
| 78 | 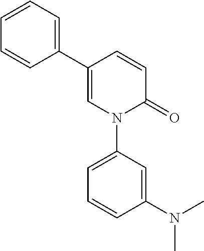 |
| 79 | 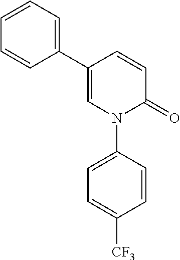 |
| 80 | 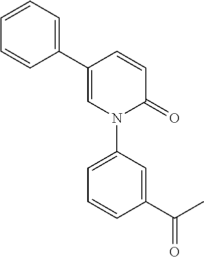 |
| 81 | 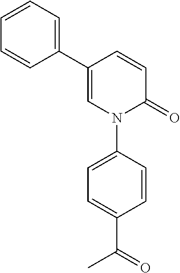 |
| 82 | 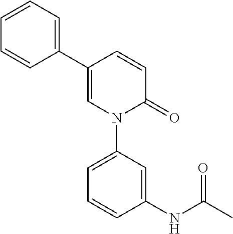 |
| 83 | 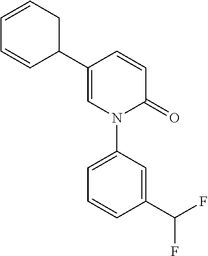 |
| 84 | 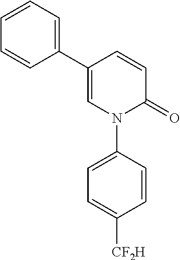 |
| 85 | 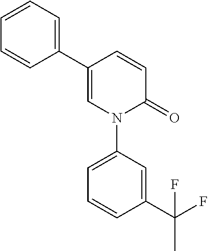 |
| 86 | 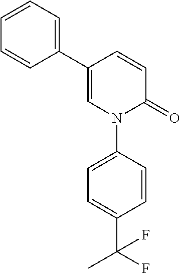 |
| 87 | 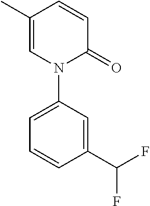 |
| 88 | 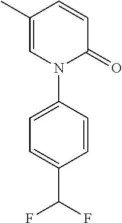 |
| 89 | 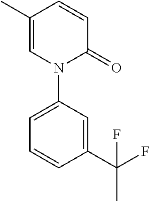 |
| 90 | 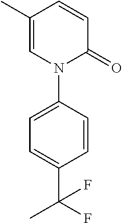 |
| 91 | 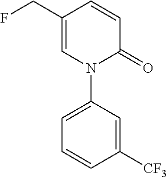 |
| 92 | 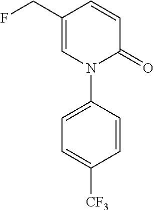 |
| 93 | 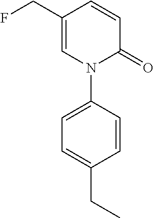 |
| 94 | 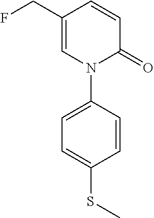 |
| 95 | 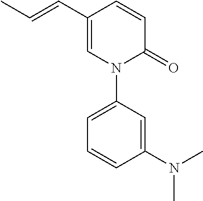 |
| 96 | 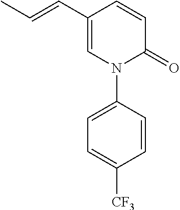 |
| 97 | 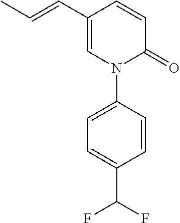 |
| 98 | 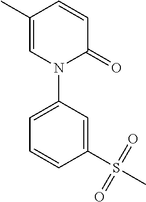 |
| 99 | 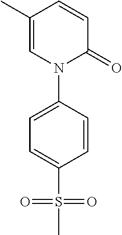 |
| 100 | 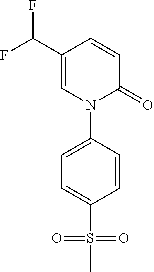 |
| 101 | 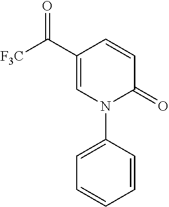 |
| 102 | 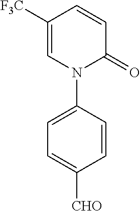 |
| 103 | 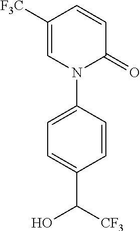 |
| 104 | 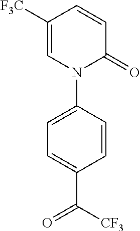 |
| 105 | 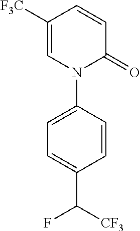 |
| 106 | 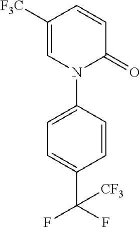 |
| 107 | 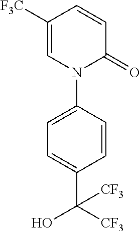 |
| 108 | 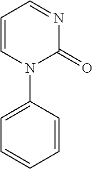 |
| 109 | 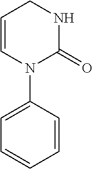 |
| 110 | 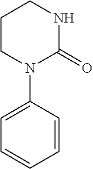 |
| 111 | 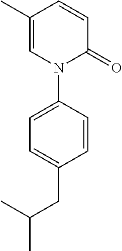 |
| 112 | 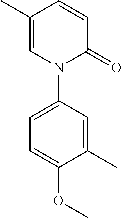 |
| 113 | 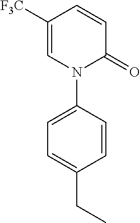 |
| 114 | 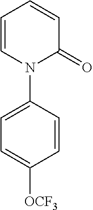 |
| 115 | 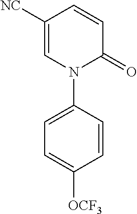 |
| 116 | 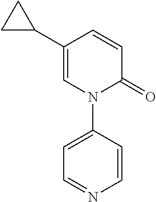 |
| 117 | 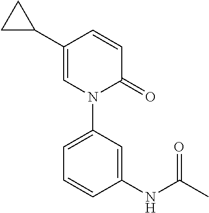 |
| 118 | 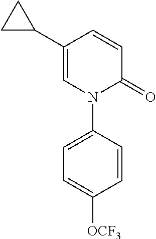 |
| 119 | 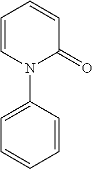 |
| 120 | 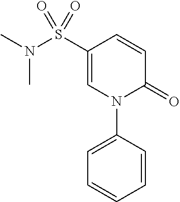 |
| 121 | 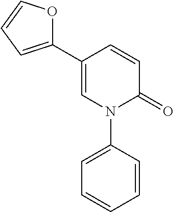 |
| 122 | 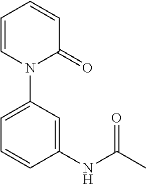 |
| 123 | 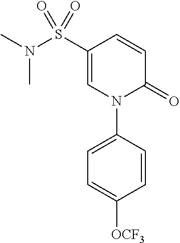 |
| 124 | 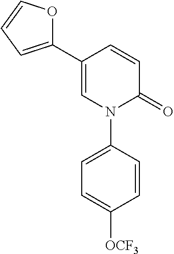 |
| 125 | 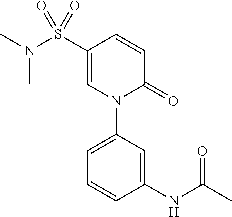 |
| 126 | 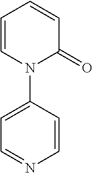 |
| 127 | 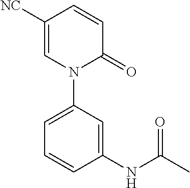 |
| 128 | 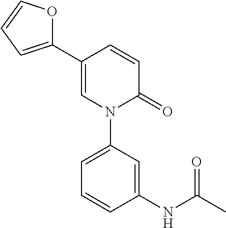 |
| 129 | 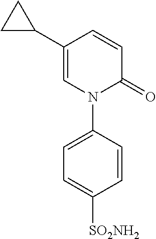 |
| 130 | 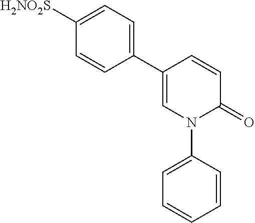 |
| 131 | 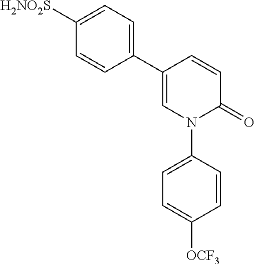 |
| 132 | 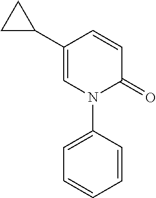 |
| 133 | 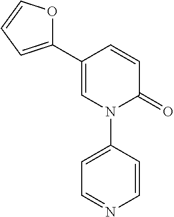 |
| 134 | 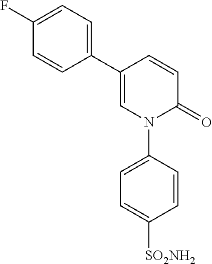 |
| 135 | 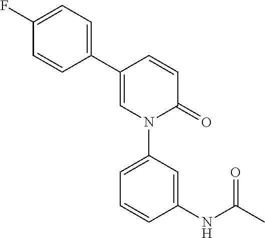 |
| 136 | 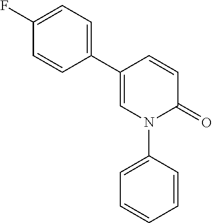 |
| 137 | 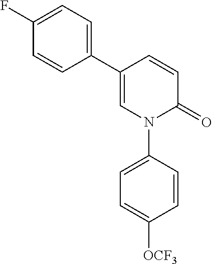 |
| 138 | 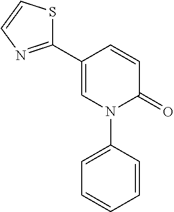 |
| 139 | 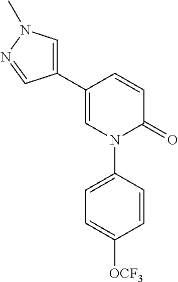 |
| 140 | 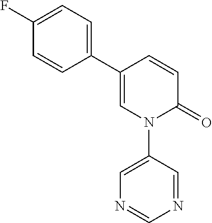 |
| 141 | 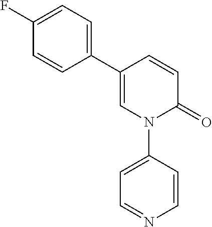 |
| 142 | 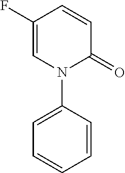 |
| 143 | 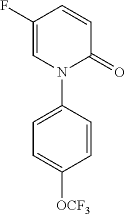 |
| 144 | 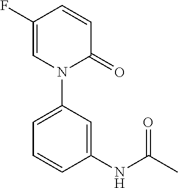 |
| 145 | 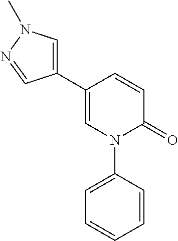 |
| 146 | 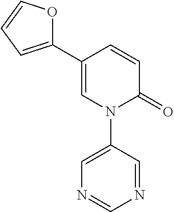 |
| 147 | 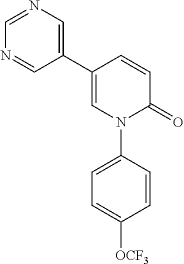 |
| 148 | 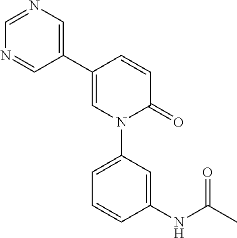 |
| 149 | 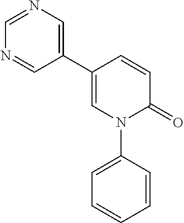 |
| 150 | 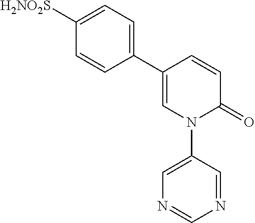 |
| 151 | 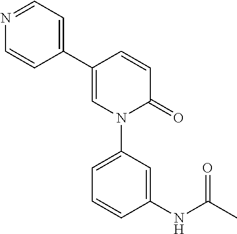 |
| 152 | 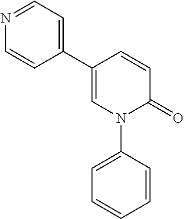 |
| 153 | 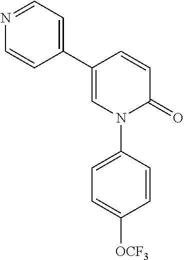 |
| 154 | 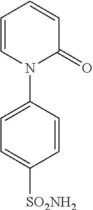 |
| 155 | 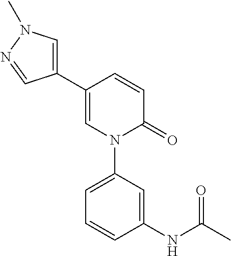 |
| 156 | 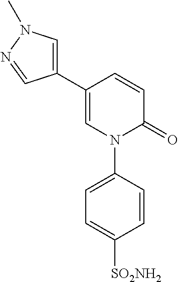 |
| 157 | 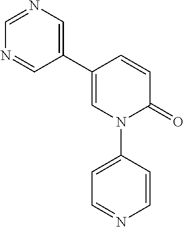 |
| 158 | 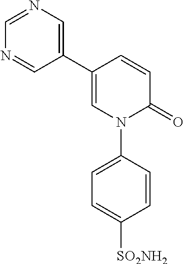 |
| 159 | 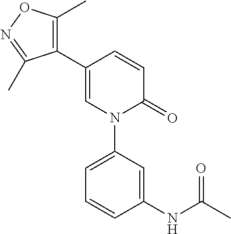 |
| 160 | 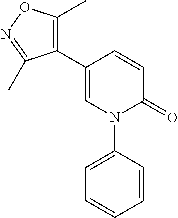 |
| 161 | 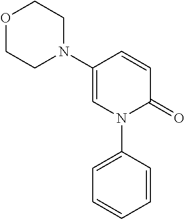 |
| 162 | 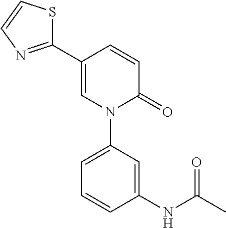 |
| 163 | 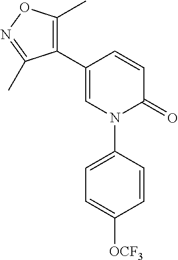 |
| 164 | 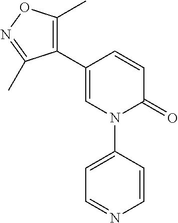 |
| 165 | 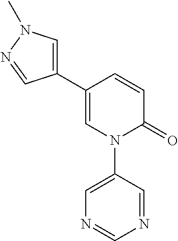 |
| 166 | 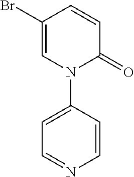 |
| 167 | 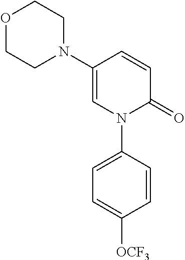 |
| 168 | 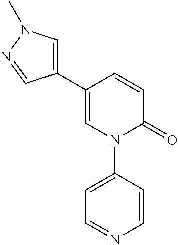 |
| 169 | 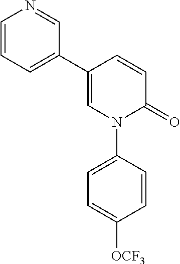 |
| 170 | 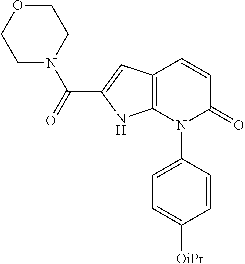 |
| 171 | 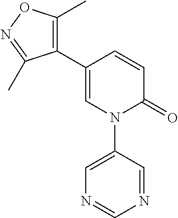 |
| 172 | 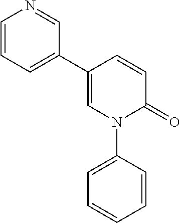 |
| 173 | 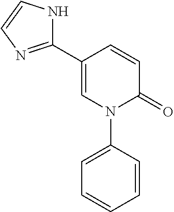 |
| 174 | 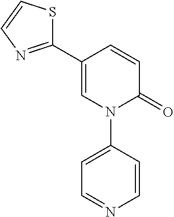 |
| 175 | 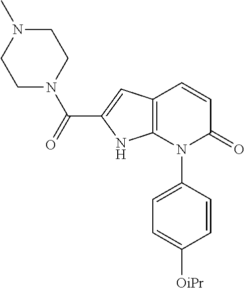 |
| 176 | 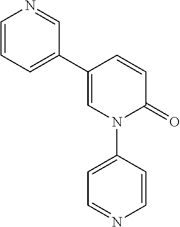 |
| 177 | 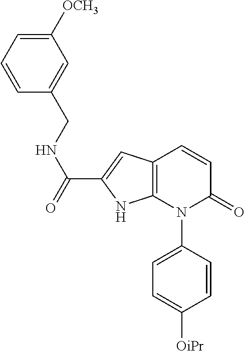 |
| 178 | 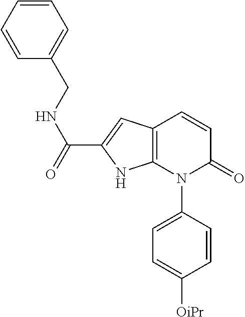 |
| 179 | 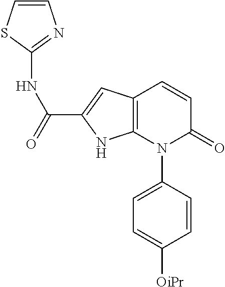 |
| 180 | 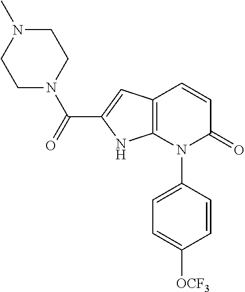 |
| 181 | 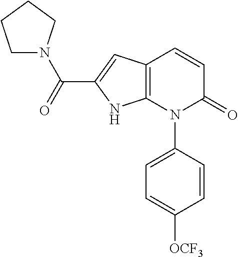 |
| 182 | 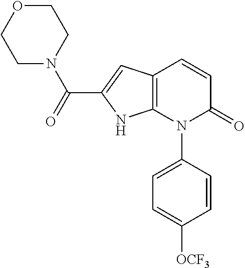 |
| 183 | 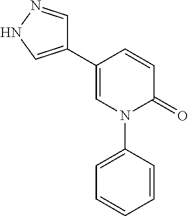 |
| 184 | 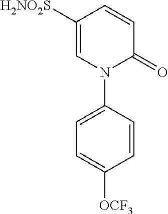 |
| 185 | 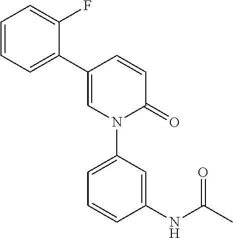 |
| 186 | 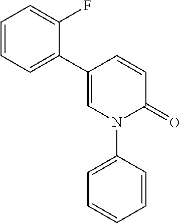 |
| 187 | 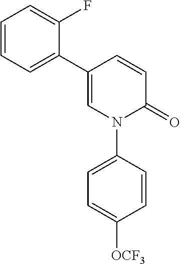 |
| 188 | 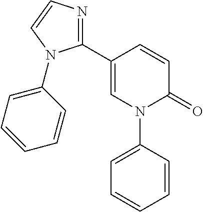 |
| 189 | 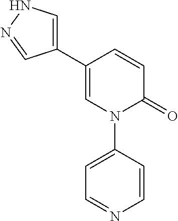 |
| 190 | 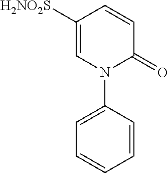 |
| 191 | 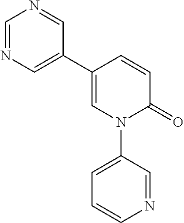 |
| 192 | 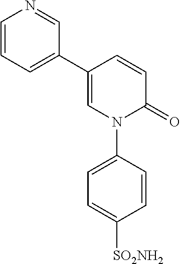 |
| 193 | 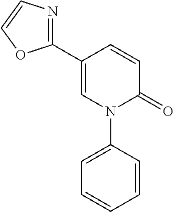 |
| 194 | 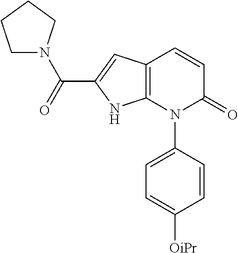 |
| 195 | 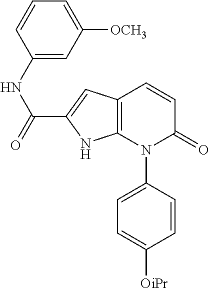 |
| 196 | 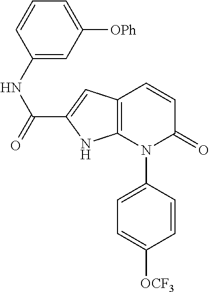 |
| 197 | 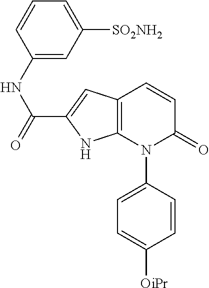 |
| 198 | 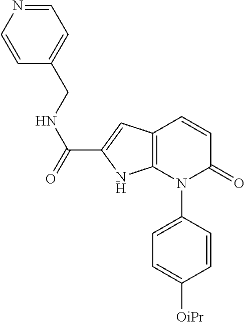 |
| 199 | 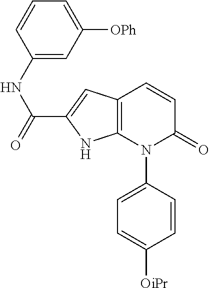 |
| 200 | 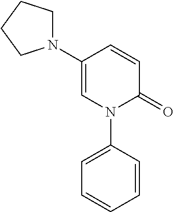 |
| 201 | 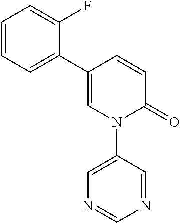 |
| 202 | 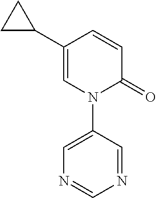 |
| 203 | 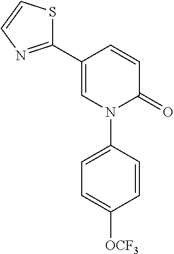 |
| 204 | 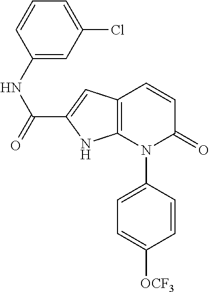 |
| 205 | 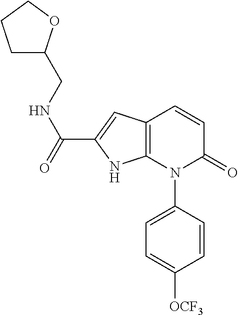 |
| 206 | 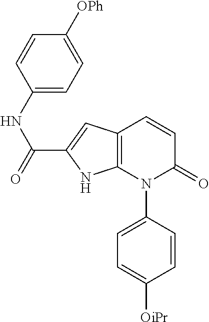 |
| 207 | 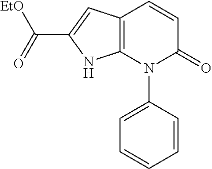 |
| 208 | 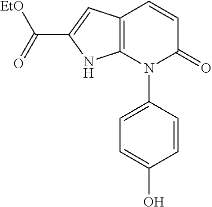 |
| 209 | 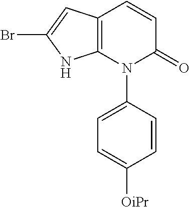 |
| 210 | 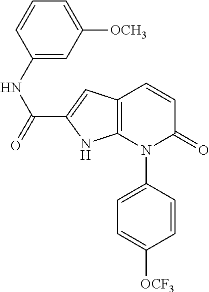 |
| 211 | 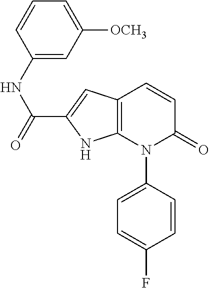 |
| 212 | 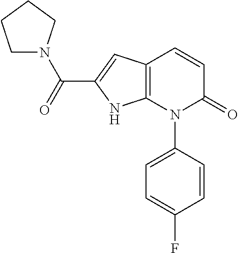 |
| 213 | 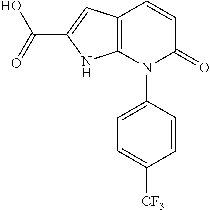 |
| 214 | 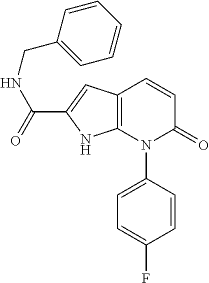 |
| 215 | 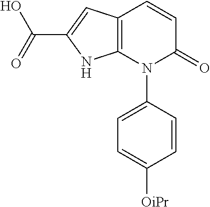 |
| 216 | 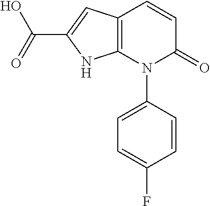 |
| 217 | 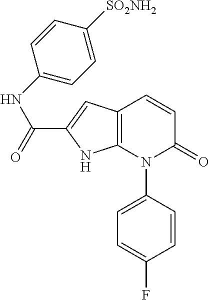 |
| 218 | 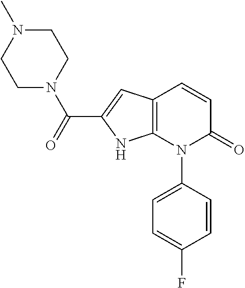 |
| 219 | 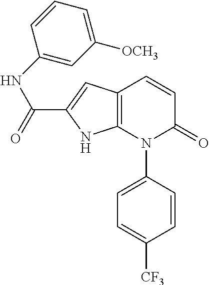 |
| 220 | 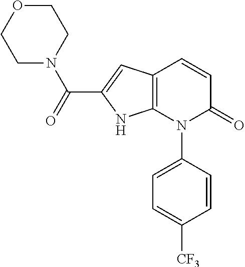 |
| 221 | 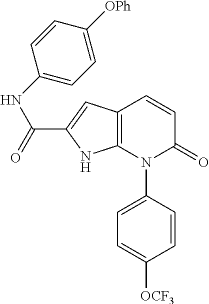 |
| 222 | 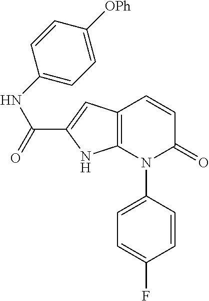 |
| 223 | 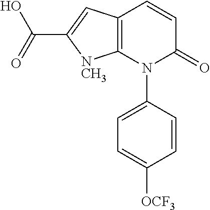 |
| 224 | 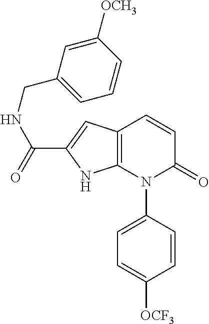 |
| 225 | 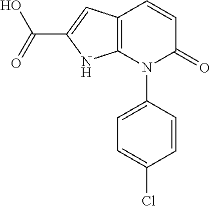 |
| 226 | 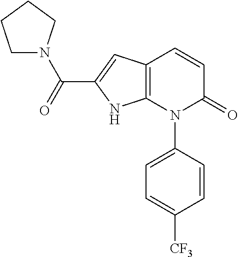 |
| 227 | 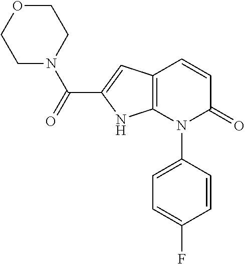 |
| 228 | 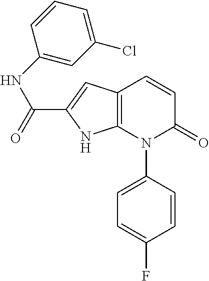 |
| 229 | 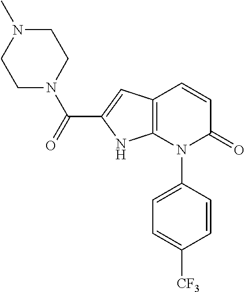 |
| 230 | 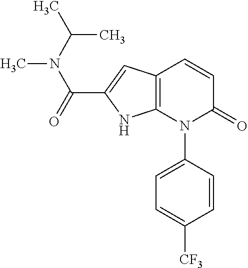 |
| 231 | 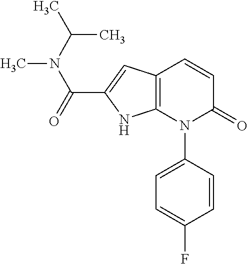 |
| 232 | 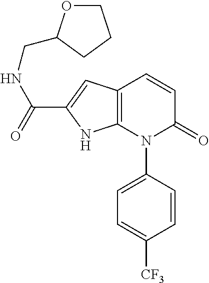 |
| 233 | 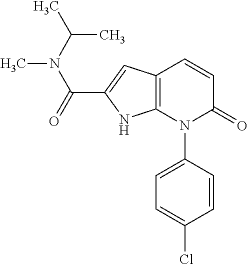 |
| 234 | 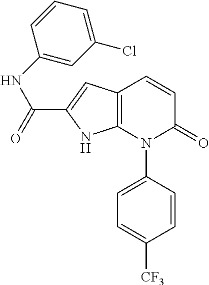 |
| 235 | 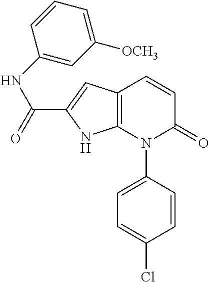 |
| 236 | 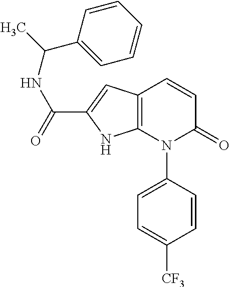 |
| 237 | 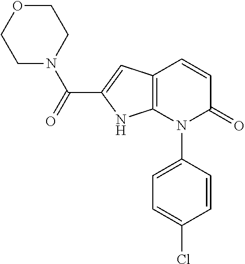 |
| 238 | 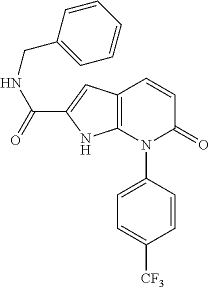 |
| 239 | 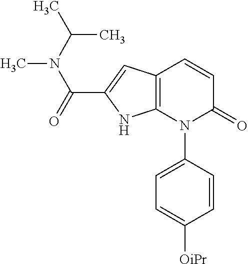 |
| 240 | 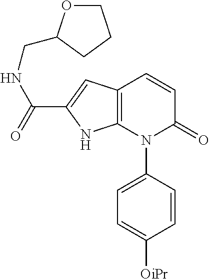 |
| 241 | 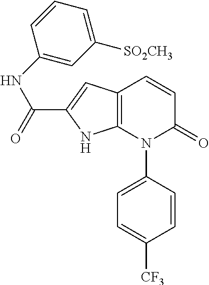 |
| 242 | 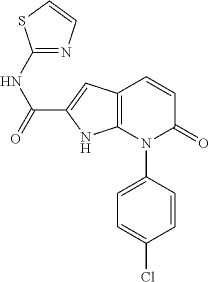 |
| 243 | 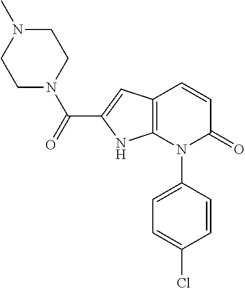 |
| 244 | 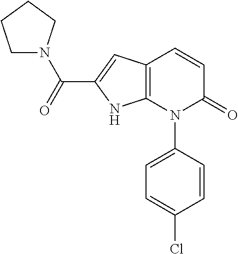 |
| 245 | 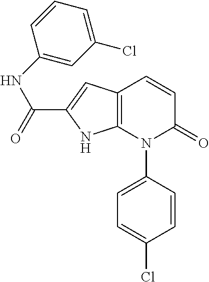 |
| 246 | 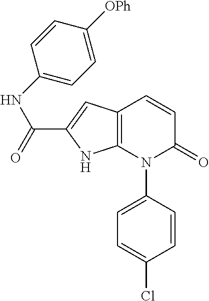 |
| 247 | 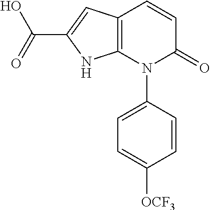 |
| 248 | 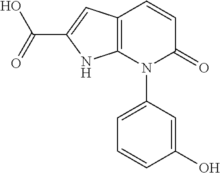 |
| 249 | 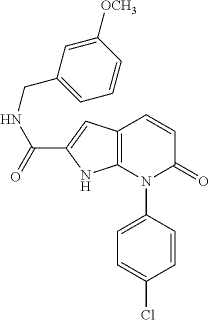 |
| 250 | 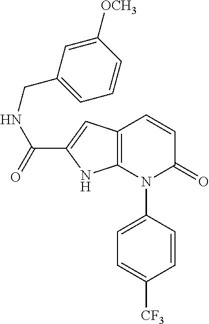 |
| 251 | 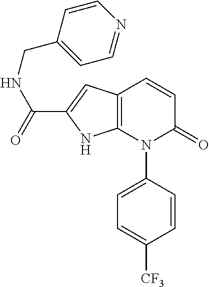 |
| 252 | 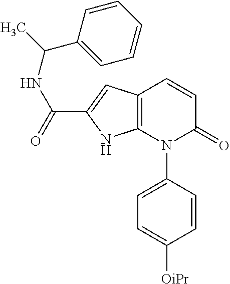 |
| 253 | 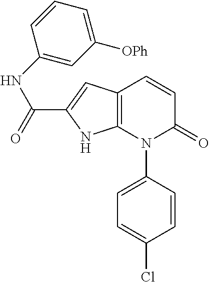 |
| 254 | 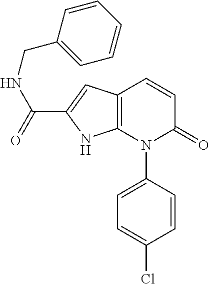 |
| 255 | 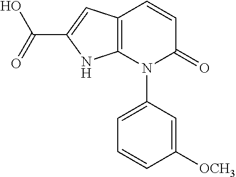 |
| 256 | 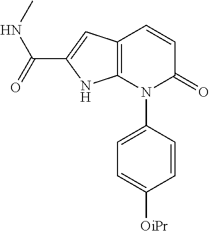 |
| 257 | 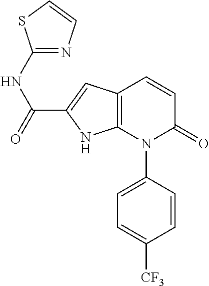 |
| 258 | 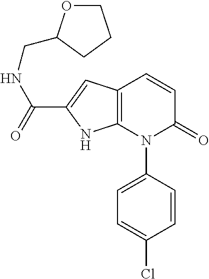 |
| 259 | 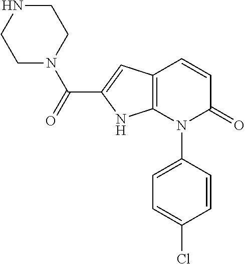 |
| 260 | 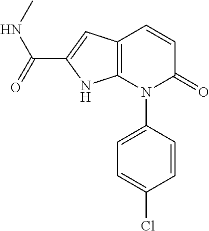 |
| 261 | 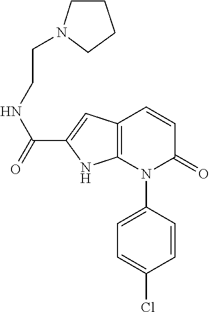 |
| 262 | 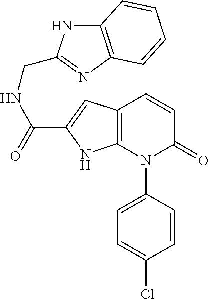 |
| 263 | 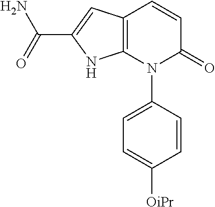 |
| 264 | 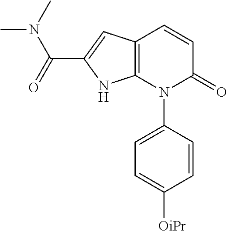 |
| 265 | 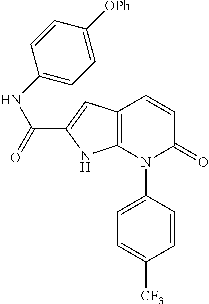 |
| 266 | 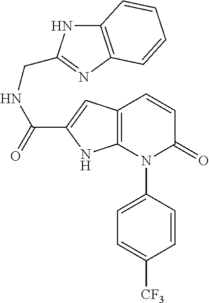 |
| 267 | 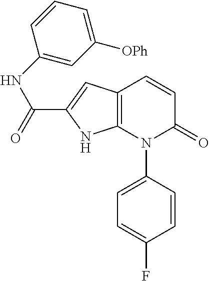 |
| 268 | 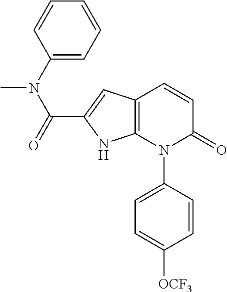 |
| 269 | 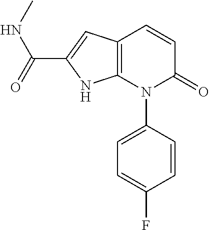 |
| 270 | 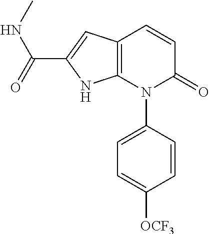 |
| 271 | 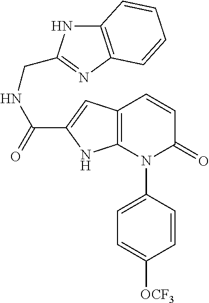 |
| 272 | 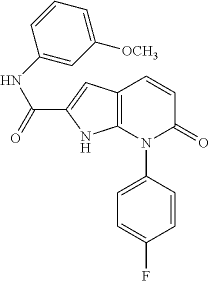 |
| 273 | 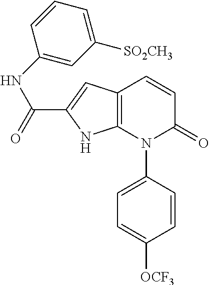 |
| 274 | 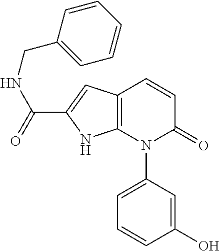 |
| 275 | 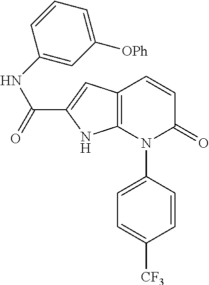 |
| 276 | 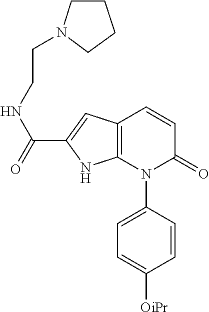 |
| 277 | 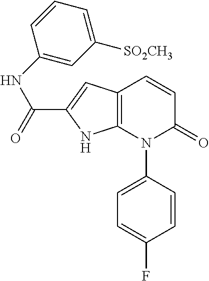 |
| 278 | 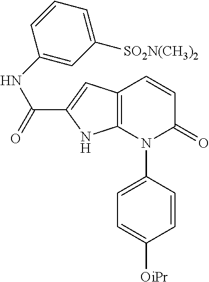 |
| 279 | 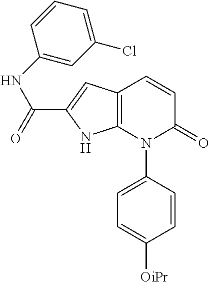 |
| 280 | 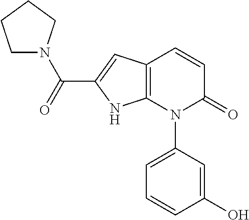 |
| 281 | 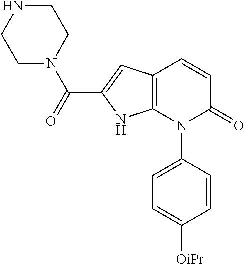 |
| 282 | 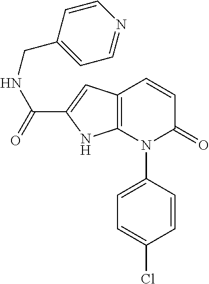 |
| 283 | 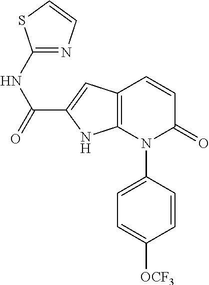 |
| 284 | 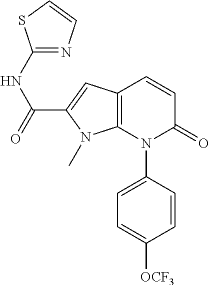 |
| 285 | 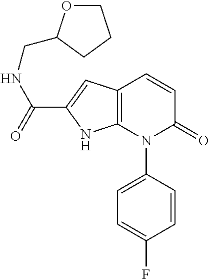 |
| 286 | 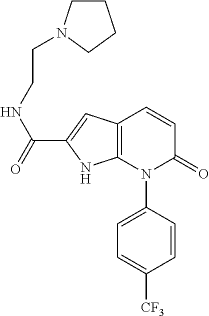 |
| 287 | 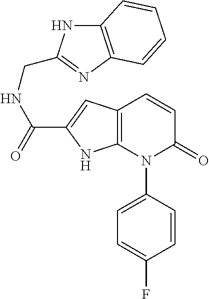 |
| 288 | 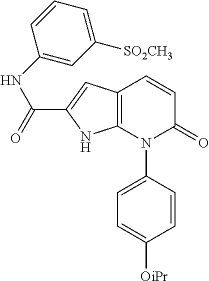 |
| 289 | 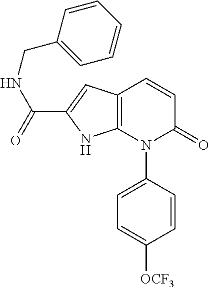 |
| 290 | 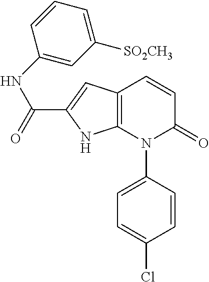 |
| 291 | 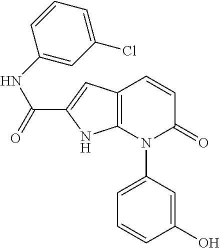 |
| 292 | 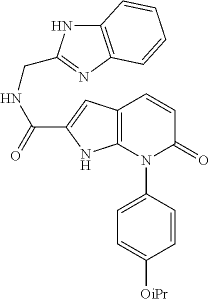 |
| 293 | 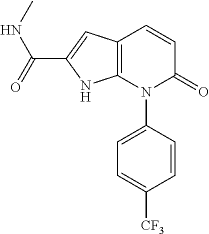 |
| 294 | 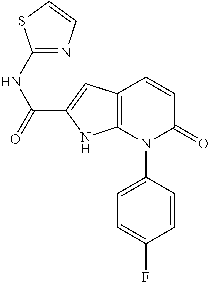 |
| 295 | 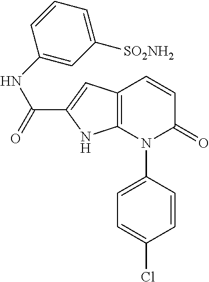 |
| 296 | 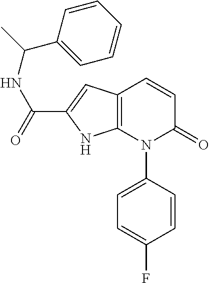 |
| 297 | 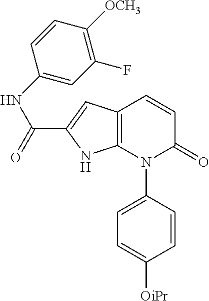 |
| 298 | 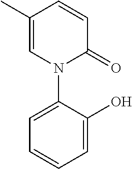 |
| 299 | 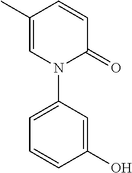 |
| 300 | 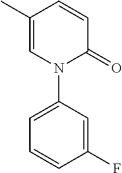 |
| 301 | 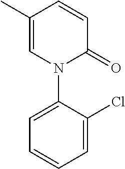 |
| 302 | 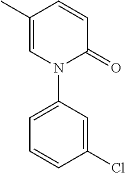 |
| 303 | 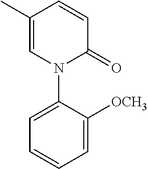 |
| 304 | 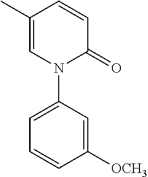 |
| 305 | 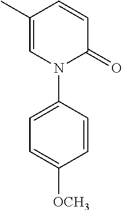 |
| 306 | 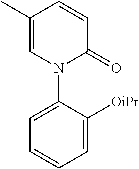 |
| 307 | 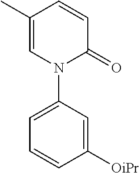 |
| 308 | 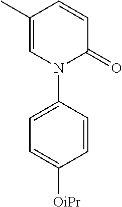 |
| 309 | 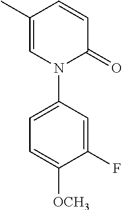 |
| 310 | 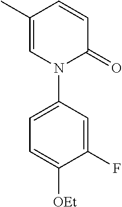 |
| 311 | 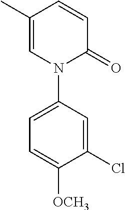 |
| 312 | 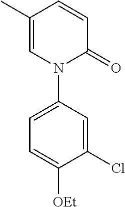 |
| 313 | 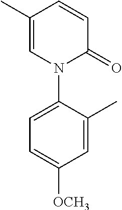 |
| 314 | 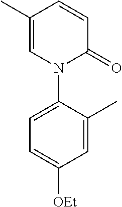 |
| 315 | 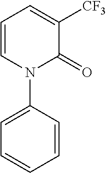 |
| 316 | 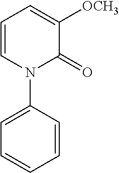 |
| 317 | 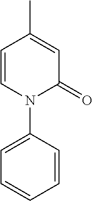 |
| 318 | 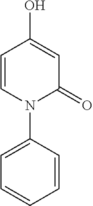 |
| 319 | 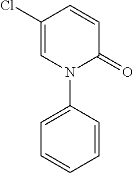 |
| 320 | 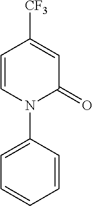 |
| 321 | 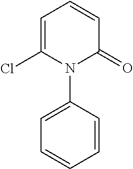 |
| 322 | 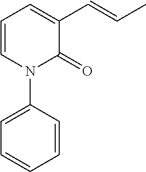 |
| 323 | 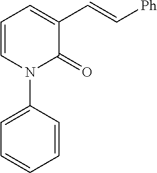 |
| 324 | 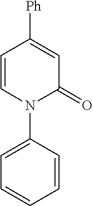 |
| 325 | 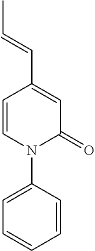 |
| 326 | 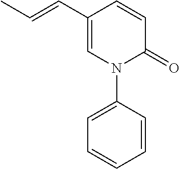 |
| 327 | 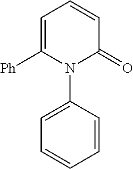 |
| 328 | 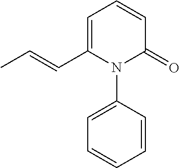 |
| 329 | 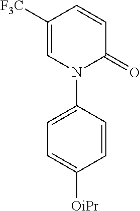 |
| 330 | 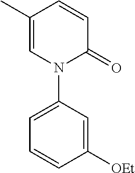 |
| 331 | 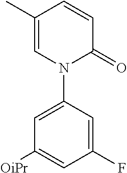 |
| 332 | 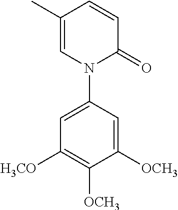 |
| 333 | 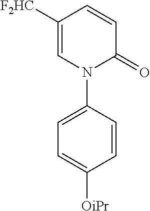 |
| 334 | 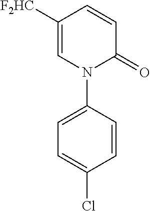 |
| 335 | 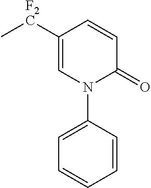 |
| 336 | 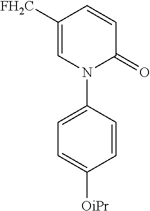 |
| 337 | 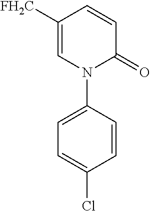 |
| 338 | 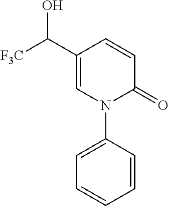 |
| 339 | 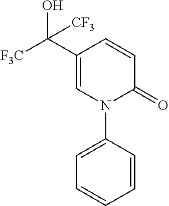 |
| 340 | 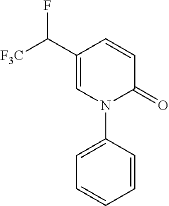 |
| 341 | 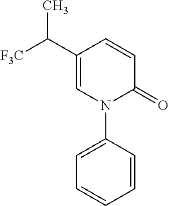 |
| 342 | 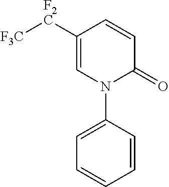 |
| 343 | 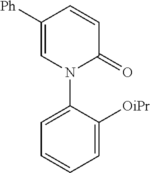 |
| 344 | 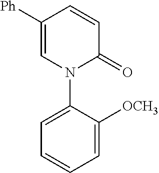 |
| 345 | 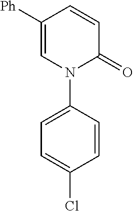 |
| 346 | 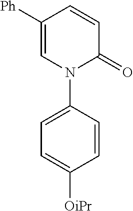 |
| 347 | 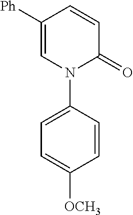 |
| 348 | 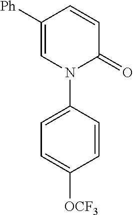 |
| 349 | 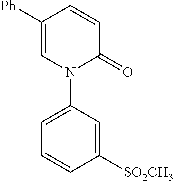 |
| 350 | 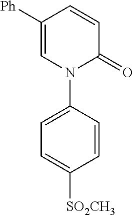 |
| 351 | 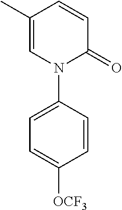 |
| 352 | 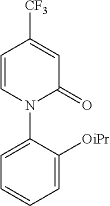 |
| 353 | 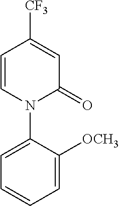 |
| 354 | 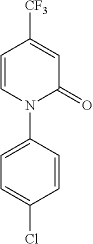 |
| 355 | 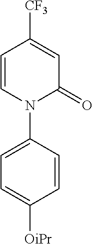 |
| 356 | 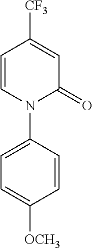 |
| 357 | 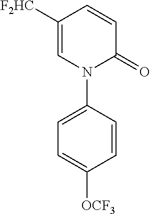 |
| 358 | 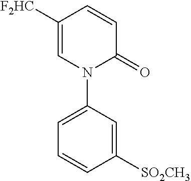 |
| 359 | 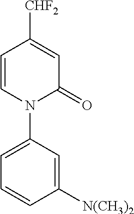 |
| 360 | 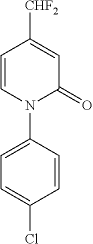 |
| 361 | 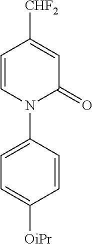 |
| 362 | 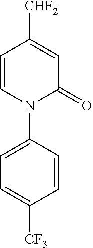 |
| 363 | 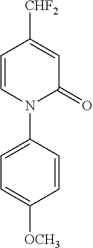 |
| 364 | 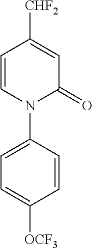 |
| 365 | 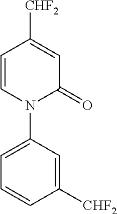 |
| 366 | 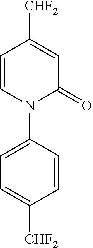 |
| 367 | 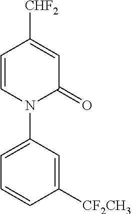 |
| 368 | 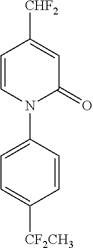 |
| 369 | 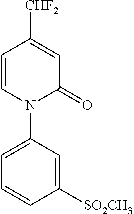 |
| 370 | 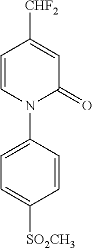 |
| 371 | 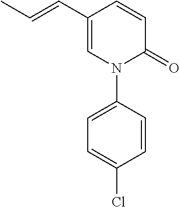 |
| 372 | 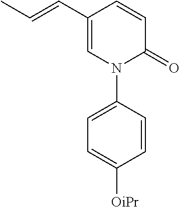 |
| 373 | 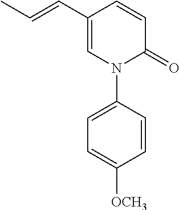 |
| 374 | 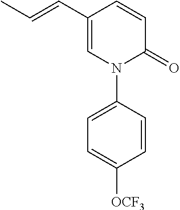 |
| 375 | Intentionally blank |
| 376 | 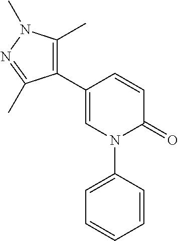 |
| 377 | 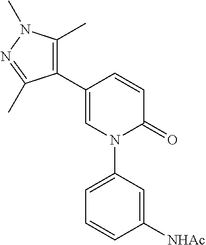 |
| 378 | 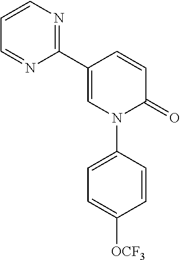 |
| 379 | 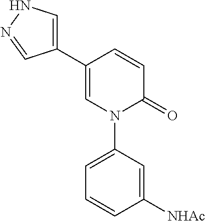 |
| 380 | 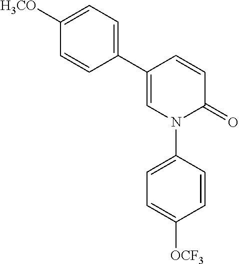 |
| 381 | 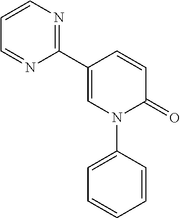 |
| 382 | 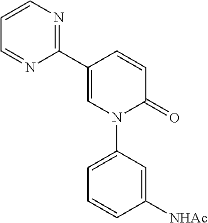 |
| 383 | 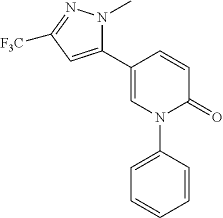 |
| 384 | 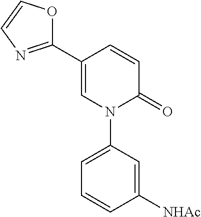 |
| 385 | 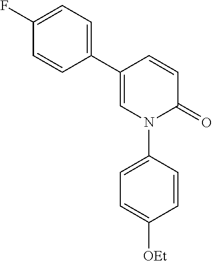 |
| 386 | 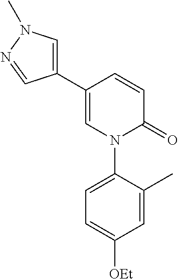 |
| 387 | 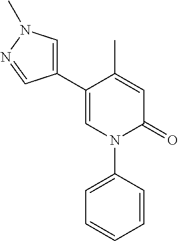 |
| 388 | 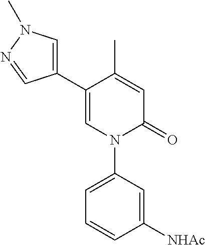 |
| 389 | 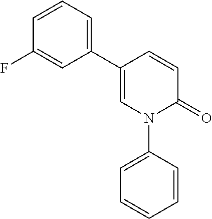 |
| 390 | 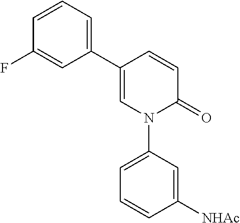 |
| 391 | 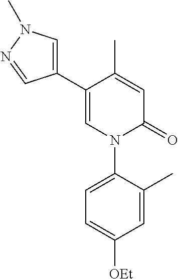 |
| 392 | 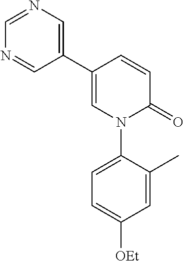 |
| 393 | 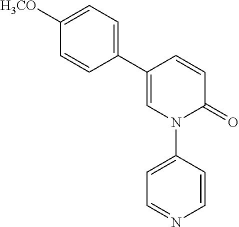 |
| 394 | 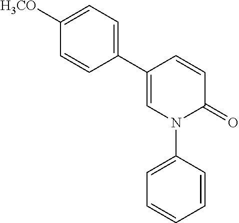 |
| 395 | 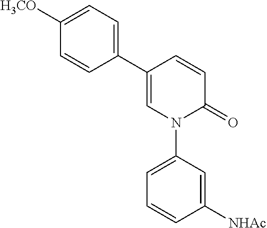 |
| 396 | 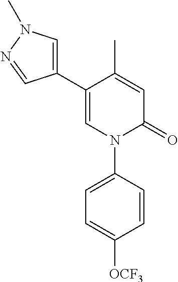 |
| 397 | 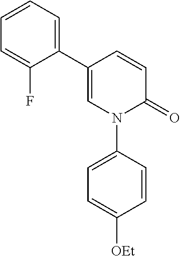 |
| 398 | 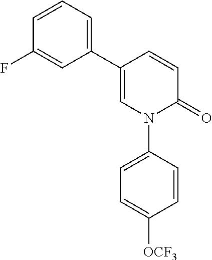 |
| 399 | 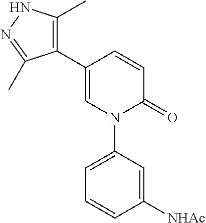 |
| 400 | 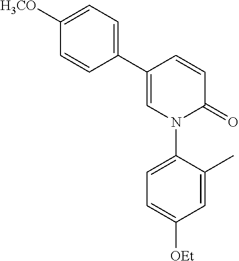 |
| 401 | 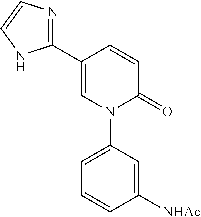 |
| 402 | 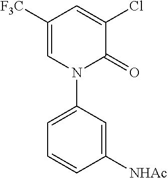 |
| 403 | 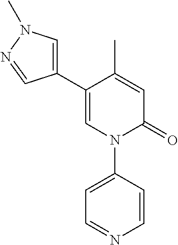 |
| 404 | 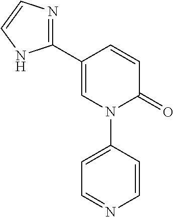 |
| 405 | 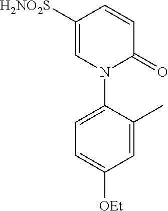 |
| 406 | 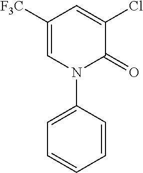 |
| 407 | 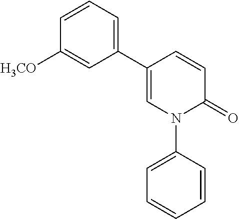 |
| 408 | 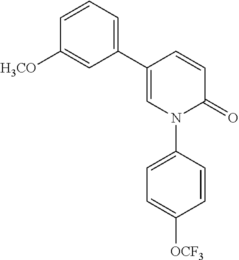 |
| 409 | 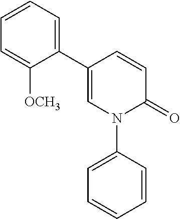 |
| 410 | 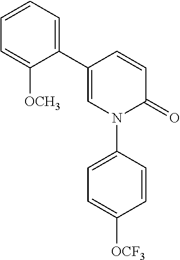 |
| 411 | 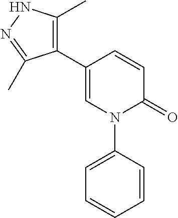 |
| 412 | 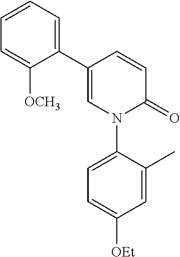 |
| 413 | 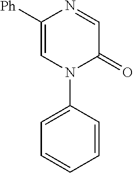 |
| 414 | 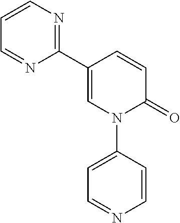 |
| 415 | 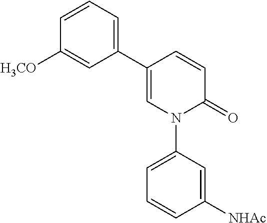 |
| 416 | 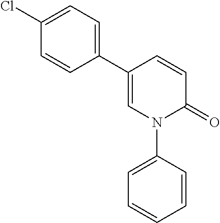 |
| 417 | 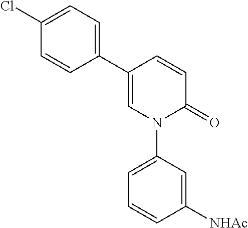 |
| 418 | 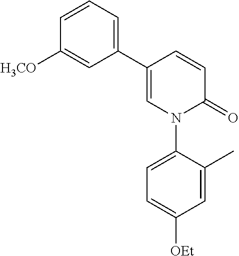 |
| 419 | 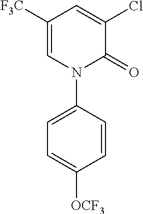 |
| 420 | 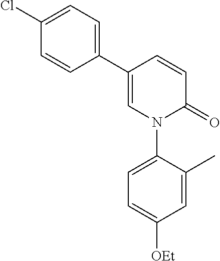 |
| 421 | 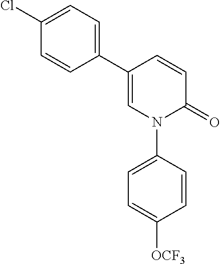 |
| 422 | 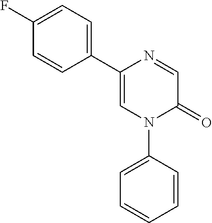 |
| 423 | 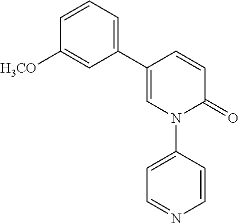 |
| 424 | 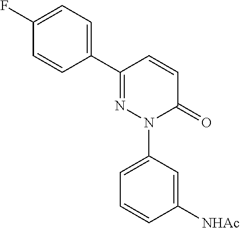 |
| 425 | 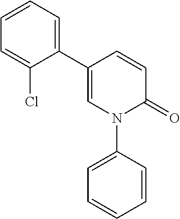 |
| 426 | 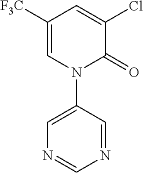 |
| 427 | 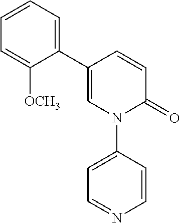 |
| 428 | 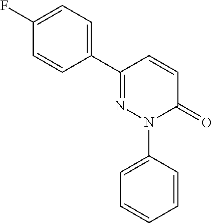 |
| 429 | 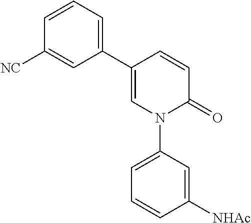 |
| 430 | 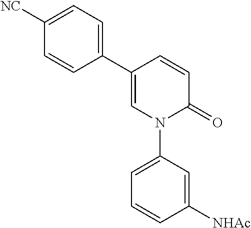 |
| 431 | 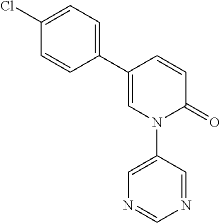 |
| 432 | 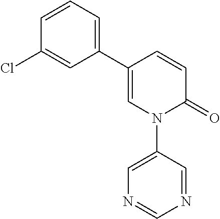 |
| 433 | 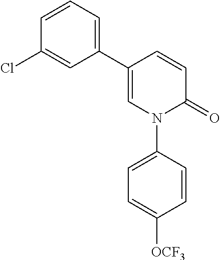 |
| 434 | 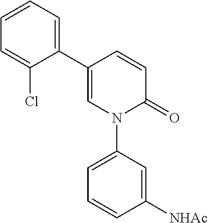 |
| 435 | 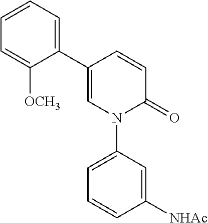 |
| 436 | 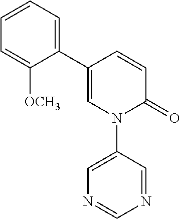 |
| 437 | 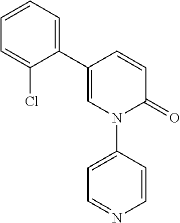 |
| 438 | 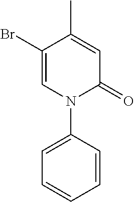 |
| 439 | 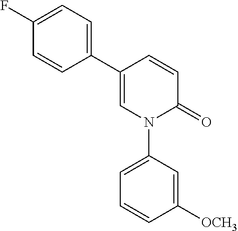 |
| 440 | 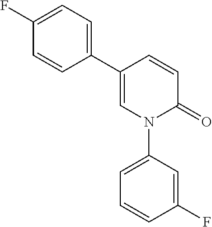 |
| 441 | 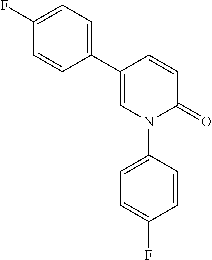 |
| 442 | 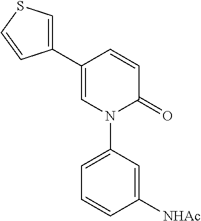 |
| 443 | 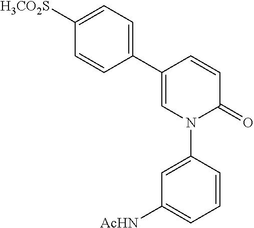 |
| 444 | 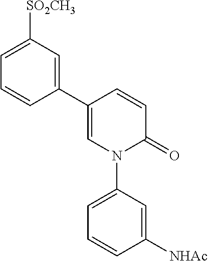 |
| 445 | 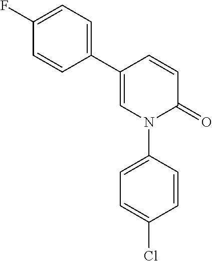 |
| 446 | 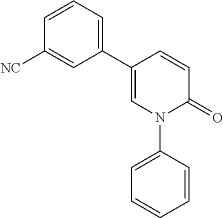 |
| 447 | 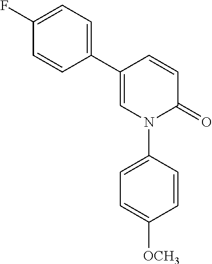 |
| 448 | 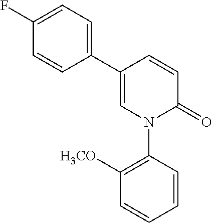 |
| 449 | 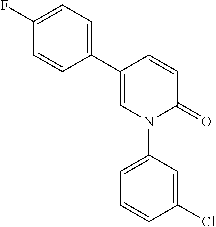 |
| 450 | 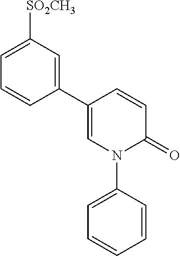 |
| 451 | 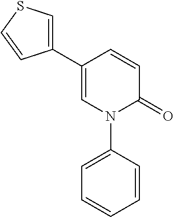 |
| 452 | 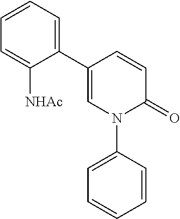 |
| 453 | 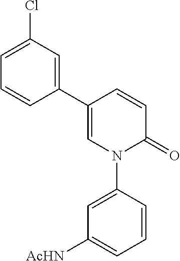 |
| 454 | 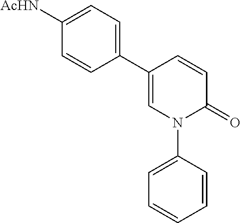 |
| 455 | 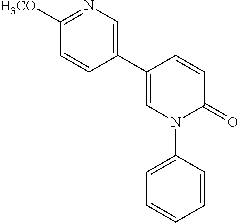 |
| 456 | 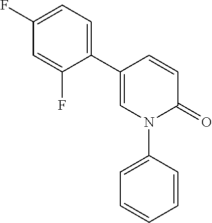 |
| 457 | 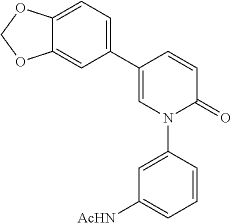 |
| 458 | 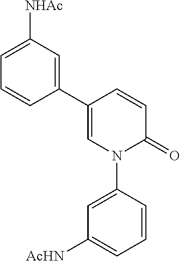 |
| 459 | 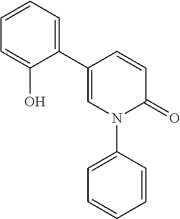 |
| 460 | 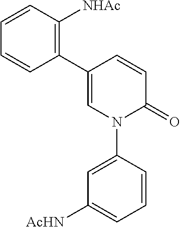 |
| 461 | 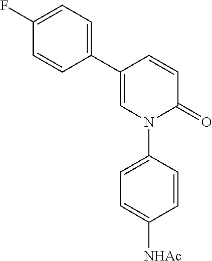 |
| 462 | 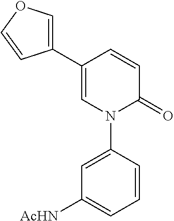 |
| 463 | 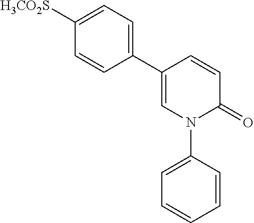 |
| 464 | 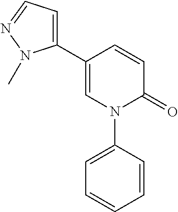 |
| 465 | 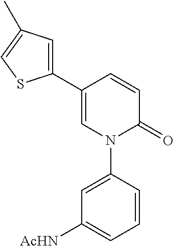 |
| 466 | 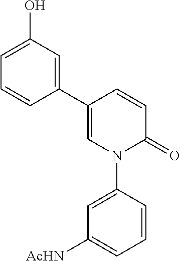 |
| 467 | 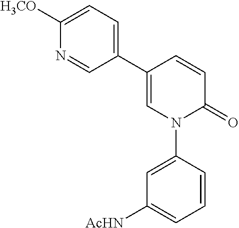 |
| 468 | 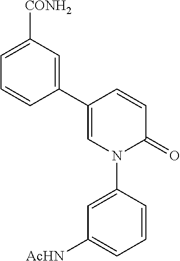 |
| 469 | 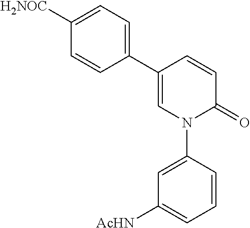 |
| 470 | 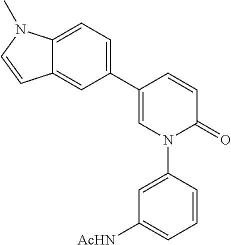 |
| 471 | 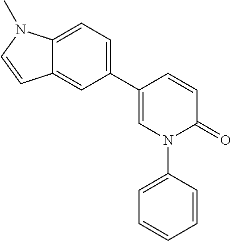 |
| 472 | 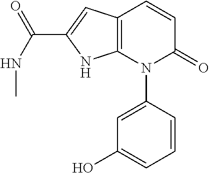 |
| 473 | 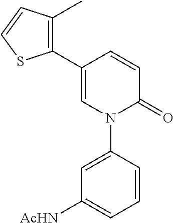 |
| 474 | 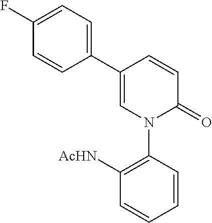 |
| 475 | 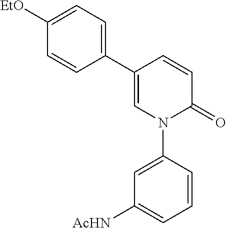 |
| 476 | 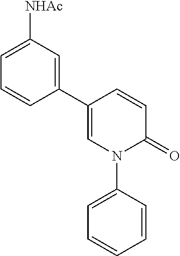 |
| 477 | 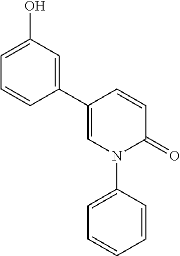 |
| 478 | 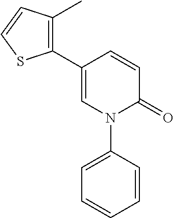 |
| 479 | 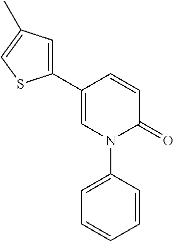 |
| 480 | 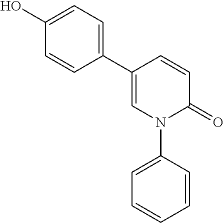 |
| 481 | 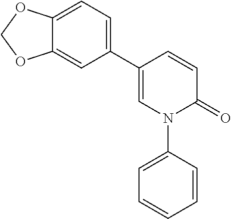 |
| 482 | 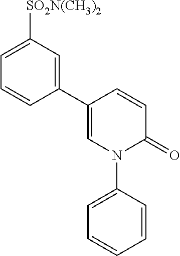 |
| 483 | 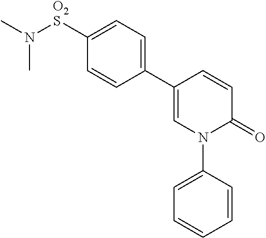 |
| 484 | 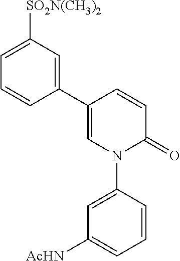 |
| 485 | 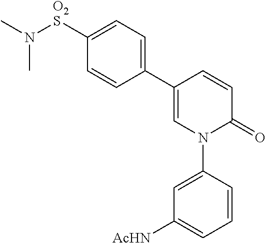 |
| 486 | 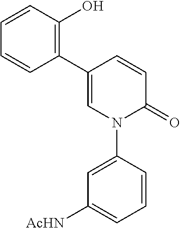 |
| 487 | 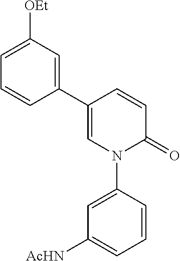 |

Other specific compounds of formula (I), (II), (III), or (IV) also include the following compounds.

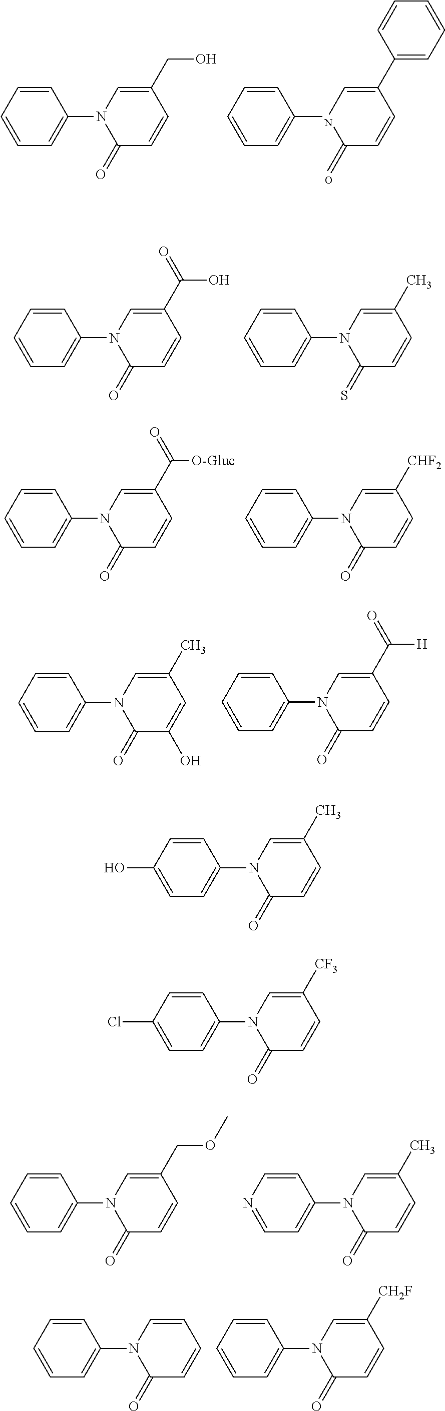

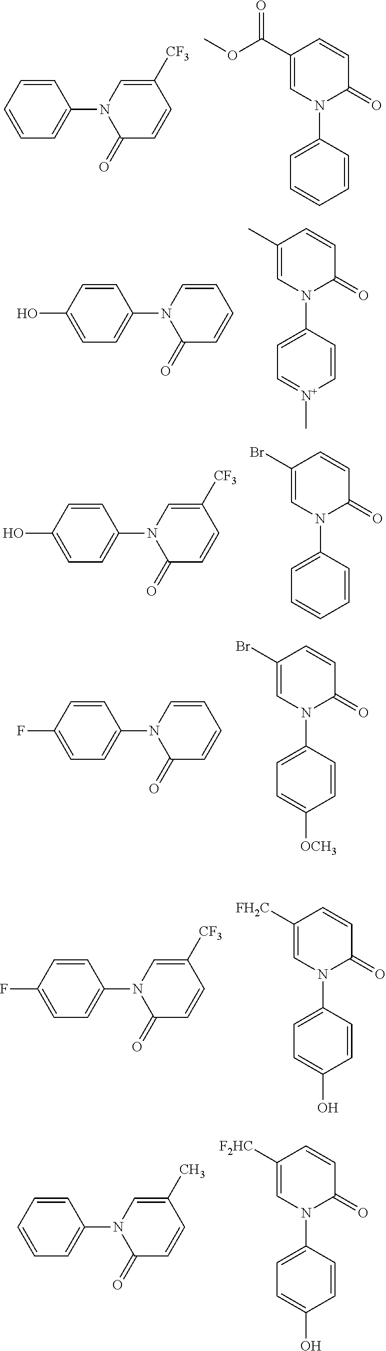

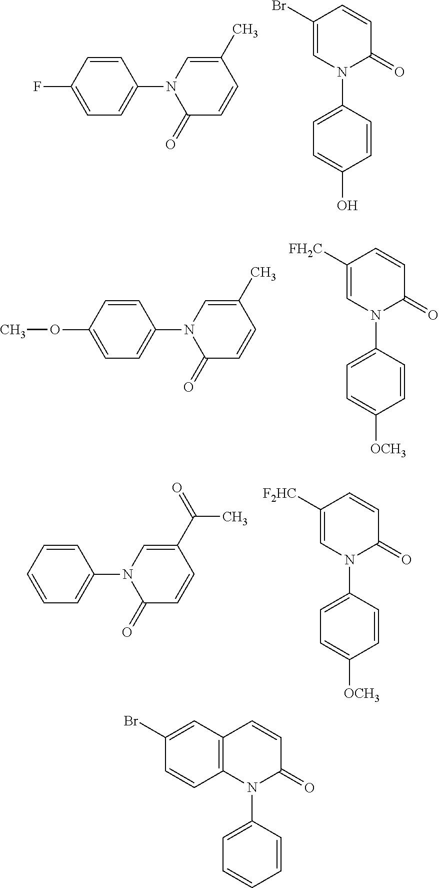

Other compounds contemplated for use in the disclosed methods include compounds of Genus I′, II′, III′, and IV′, below. Synthesis of compounds of Genus I′, II′, III′, and IV′ are described in detail in International Patent Publication No. WO 07/062,167, incorporated by reference in its entirety herein.

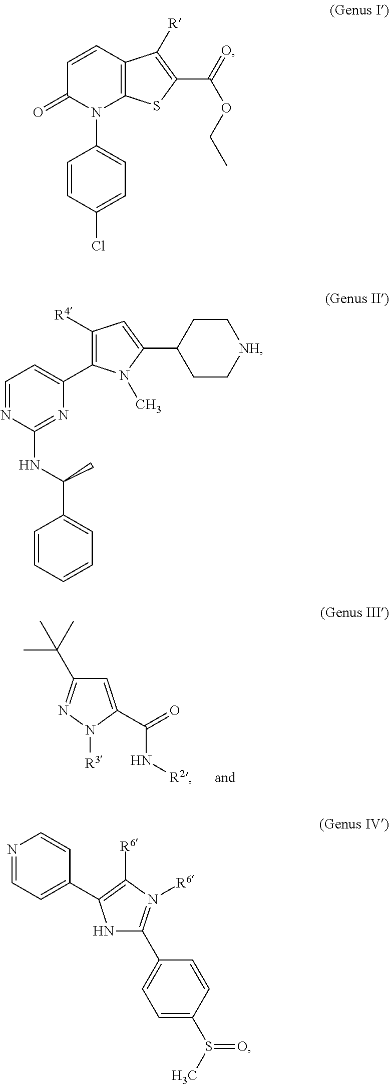

wherein each of R′, R^{2′}, R^{3′}, R^{4′}, and R^{6′} is independently selected from the group consisting of H, halo, cyano, nitro, hydroxy, optionally substituted C₁₋₆ alkyl, optionally substituted C₃₋₇ cycloalkyl, optionally substituted C₄₋₁₀ alkylcycloalkyl, optionally substituted C₂₋₆ alkenyl, optionally substituted C₁₋₆ alkoxy, optionally substituted C_{6 or 10} aryl, optionally substituted pyridinyl, optionally substituted pyrimidinyl, optionally substituted thienyl, optionally substituted furanyl, optionally substituted thiazolyl, optionally substituted oxazolyl, optionally substituted phenoxy, optionally substituted thiophenoxy, optionally substituted sulphonamido, optionally substituted urea, optionally substituted thiourea, optionally substituted amido, optionally substituted keto, optionally substituted carboxyl, optionally substituted carbamyl, optionally substituted sulphide, optionally substituted sulphoxide, optionally substituted sulphone, optionally substituted amino, optionally substituted alkoxyamino, optionally substituted alkyoxyheterocyclyl, optionally substituted alkylamino, optionally substituted alkylcarboxy, optionally substituted carbonyl, optionally substituted spirocyclic cycloalkyl, optionally substituted pyrazinyl, optionally substituted pyridazinyl, optionally substituted pyrrolyl, optionally substituted thiophenyl, optionally substituted thiazolyl, optionally substituted oxazolyl, optionally substituted imidazolyl, optionally substituted isoxazolyl, optionally substituted pyrazolyl, optionally substituted isothiazolyl, optionally substituted napthyl, optionally substituted quinolinyl, optionally substituted isoquinolinyl, optionally substituted quinoxalinyl, optionally substituted benzothiazolyl, optionally substituted benzothiophenyl, optionally substituted benzofuranyl, optionally substituted indolyl, and optionally substituted benzimidazolyl, or a pharmaceutically acceptable salt, ester, solvate or prodrug thereof.

The salts, e.g., pharmaceutically acceptable salts, of the disclosed therapeutics may be prepared by reacting the appropriate base or acid with a stoichiometric equivalent of the therapeutic. Similarly, pharmaceutically acceptable derivatives (e.g., esters), metabolites, hydrates, solvates and prodrugs of the therapeutic may be prepared by methods generally known to those skilled in the art. Thus, another embodiment provides compounds that are prodrugs of an active compound. In general, a prodrug is a compound which is metabolized in vivo (e.g., by a metabolic transformation such as deamination, dealkylation, de-esterification, and the like) to provide an active compound. A “pharmaceutically acceptable prodrug” means a compound which is, within the scope of sound medical judgment, suitable for pharmaceutical use in a patient without undue toxicity, irritation, allergic response, and the like, and effective for the intended use, including a pharmaceutically acceptable ester as well as a zwitterionic form, where possible, of the therapeutic. As used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates. Examples of pharmaceutically-acceptable prodrug types are described in Higuchi and Stella, Pro-drugs as Novel Delivery Systems, Vol. 14 of the A. C. S. Symposium Series, and in Roche, ed., Bioreversible Carriers in Drug Design, American Pharmaceutical Association and Pergamon Press, 1987, both of which are incorporated herein by reference.

The compounds and compositions described herein may also include metabolites. As used herein, the term “metabolite” means a product of metabolism of a compound of the embodiments or a pharmaceutically acceptable salt, analog, or derivative thereof, that exhibits a similar activity in vitro or in vivo to a disclosed therapeutic. The compounds and compositions described herein may also include hydrates and solvates. As used herein, the term “solvate” refers to a complex formed by a solute (herein, the therapeutic) and a solvent. Such solvents for the purpose of the embodiments preferably should not negatively interfere with the biological activity of the solute. Solvents may be, by way of example, water, ethanol, or acetic acid. In view of the foregoing, reference herein to a particular compound or genus of compounds will be understood to include the various forms described above, including pharmaceutically acceptable salts, esters, prodrugs, metabolites and solvates thereof.

### Dosing and Pharmaceutical Formulations

The terms “therapeutically effective amount” and “prophylactically effective amount,” as used herein, refer to an amount of a compound sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by any of the assays or clinical diagnostic tests described herein. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

The therapeutics disclosed herein can be dosed at a total amount of about 50 to about 2400 mg per day. The dosage can be divided into two or three doses over the day or given in a single daily dose. Specific amounts of the total daily amount of the therapeutic contemplated for the disclosed methods include about 50 mg, about 100 mg, about 150 mg, about 200 mg, about 250 mg, about 267 mg, about 300 mg, about 350 mg, about 400 mg, about 450 mg, about 500 mg, about 534 mg, about 550 mg, about 600 mg, about 650 mg, about 700 mg, about 750 mg, about 800 mg, about 850 mg, about 900 mg, about 950 mg, about 1000 mg, about 1050 mg, about 1068 mg, about 1100 mg, about 1150 mg, about 1200 mg, about 1250 mg, about 1300 mg, about 1335 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1869 mg, about 1900 mg, about 1950 mg, about 2000 mg, about 2050 mg, about 2100 mg, about 2136 mg, about 2150 mg, about 2200 mg, about 2250 mg, about 2300 mg, about 2350 mg, and about 2400 mg.

Dosages of the therapeutic can alternately be administered as a dose measured in mg/kg. Contemplated mg/kg doses of the disclosed therapeutics include about 1 mg/kg to about 60 mg/kg. Specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg.

In methods where the patient has suffered an AMI, administration of the therapeutic can be initiated at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days after suffering the AMI. Also contemplated is initiation of the treatment about 1-40 days, about 1-30 days, about 1-25 days, about 1-20 days, about 1-14 days, about 1-10 days, about 2-40 days, about 3-40 days, about 3-38 days, about 3-30 days, about 3-25 days, about 3-20 days, about 3-15 days, about 3-14 days, about 3-10 days, about 4-36 days, about 4-30 days, about 4-25 days, about 4-20 days, about 4-14 days, about 5-40 days, about 5-34 days, about 5-30 days, about 5-25 days, about 5-20 days, about 5-14 days, about 6-40 days, about 6-32 days, about 6-30 days, about 6-25 days, about 6-20 days, about 6-14 days, about 7-40 days, about 7-30 days, about 7-25 days, about 7-20 days, about 7-14 days, about 8-28 days, about 9-26 days, about 10-24 days, about 12-22 days, about 13-20 days, or about 14-18 days after suffering the AMI. Treatment, e.g., continued administration of the therapeutic can continue for at least a week, at least 2 weeks, at least 3 weeks, at least a month, at least 6 weeks, at least 2 months, at least 3 months, at least 4 months, at least 5 months, at least 6 months, or at least a year. For example, the treatment can be for up to 3 months, up to 4 months, up to 5 months, or up to 6 months. In some embodiments, a patient suffering an AMI continues to be administered the therapeutic for a time period up to 4 weeks after suffering the AMI, e.g., the therapeutic continues to be administered on the day that is 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, and/or 28 days after suffering the AMI.

As described elsewhere herein, the compounds described herein may be formulated in pharmaceutical compositions with a pharmaceutically acceptable excipient, carrier, or diluent. The compound or composition comprising the compound can be administered by any route that permits treatment of the disease or condition. A preferred route of administration is oral administration. Additionally, the compound or composition comprising the compound may be delivered to a patient using any standard route of administration, including parenterally, such as intravenously, intraperitoneally, intrapulmonary, subcutaneously or intramuscularly, intrathecally, transdermally, rectally, orally, nasally or by inhalation. Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or the compound of the embodiments can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of bacterial infection (e.g., anti-bacterial or anti-microbial agents). In various embodiments, examples of a therapeutic agent that may be used alone or in combination with another therapeutic agent according to the methods of the present invention include, but are not limited to, an agent that reduces tissue remodeling or fibrosis, reduces the activity of transforming growth factor-beta (TGF-β), targets one or more TGF-β isoforms, inhibits TGF-β receptor kinases TGFBR1 (ALK5) and/or TGFBR2, or modulates one or more post-receptor signaling pathways, is an endothelin receptor antagonists, targets both endothelin receptor A and endothelin receptor B or selectively targets endothelin receptor A, reduces activity of connective tissue growth factor (CTGF), inhibits matrix metalloproteinase (MMP), particularly MMP-9 and/or MMP-12, reduces the activity of epidermal growth factor receptor (EGFR), targets the EGF receptor, or inhibits EGF receptor kinase, reduces the activity of platelet derived growth factor (PDGF), targets PDGF receptor (PDGFR), inhibits PDGFR kinase activity, or inhibits post-PDGF receptor signaling pathways, reduces the activity of vascular endothelial growth factor (VEGF), targets one or more of VEGF receptor 1 (VEGFR1, Flt-1), VEGF receptor 2 (VEGFR2, KDR), inhibits multiple receptor kinases as in the case of BIRB-1120 which inhibits receptor kinases for vascular endothelial growth factor, fibroblast growth factor, and platelet derived growth factor, interferes with integrin function, particularly integrin αVβ6, interferes with pro-fibrotic activities of IL-4 and IL-13, targets IL-4 receptor, IL-13 receptor, modulates signaling though the JAK-STAT kinase pathway, interferes with epithelial mesenchymal transition, inhibits mTor, reduces levels of copper, reduces oxidative stress, inhibits prolyl hydrolase, inhibits phosphodiesterase 4 (PDE4) or phosphodiesterase 5 (PDE5), modifies the arachidonic acid pathway, or acts as an agonist of PPAR-γ.

Formulations, e.g., for parenteral or oral administration, are most typically solids, liquid solutions, emulsions or suspensions, while inhalable formulations for pulmonary administration are generally liquids or powders, with powder formulations being generally preferred. A preferred pharmaceutical composition may also be formulated as a lyophilized solid that is reconstituted with a physiologically compatible solvent prior to administration. Alternative pharmaceutical compositions may be formulated as syrups, creams, ointments, tablets, and the like.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The pharmaceutical compositions described herein may be formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, the active compound is dissolved in DMSO and diluted with water.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In a preferred embodiment, the compounds described herein may be formulated for oral administration in a lipid-based formulation suitable for low solubility compounds. Lipid-based formulations can generally enhance the oral bioavailability of such compounds.

As such, a preferred pharmaceutical composition comprises a therapeutically or prophylactically effective amount of a compound described herein, together with at least one pharmaceutically acceptable excipient selected from the group consisting of medium chain fatty acids and propylene glycol esters thereof (e.g., propylene glycol esters of edible fatty acids, such as caprylic and capric fatty acids) and pharmaceutically acceptable surfactants, such as polyoxyl 40 hydrogenated castor oil.

In an alternative preferred embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the invention in the composition.

The methods of the embodiments also include the use of a compound or compounds as described herein together with one or more additional therapeutic agents for the treatment of disease conditions. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### Example 1

### Experimental Myocardial Infarction (MI) Protocol

In this example, a protocol is described for examining the ventricular function, extent of fibrosis and VT inducibility in an ischemia-reperfusion rat model after pirfenidone treatment. Ventricular function was assessed via echocardiography. VT inducibility was assessed by programmed stimulation and EP study. The electrophysiological properties were assessed using high-resolution optical mapping, and the extent of fibrosis was studied using standard histological techniques.

After baseline echocardiography, thirty male Sprague-Dawley rats, ages 6-10 weeks, underwent myocardial infarction using an ischemia-reperfusion model. Briefly, rats were anesthetized using inhaled isoflurane (5% induction, 2.5% maintenance, O2 output 1 L/min) and positioned supine on an electrically warmed animal surgery platform. Rats were intubated using a 16-gauge i.v. catheter and then ventilated using a Harvard rodent respirator. After a left thoracotomy and pericardiotomy were performed, a 7-0 Ticron suture was introduced into the myocardium, using the left atrial appendage and right outflow tract as landmarks. The depth of entry was 2 mm, which was slightly greater than the level of the left coronary artery. Both suture ends were then threaded through a PE-90 polyethylene tube 6 in. in length to form a “snare loop” around the artery, closed by pulling on the free ends of the suture. The snare loop was tested by closing and releasing after 10-seconds to demonstrate adequate ischemia and reperfusion. The suture was then tightened to occlude the artery for 20 minutes and then removed to allow for reperfusion. The chest was then closed with 5-0 prolene suture, and the animal was allowed to recover. After one week and repeat echocardiography, rats were randomized to placebo rodent feed (control group, n=15) or rodent feed mixed with 1.2% pirfenidone (PFD) (treatment group, n=15) for four weeks. All experiments and data analyses were performed with the operator blinded to treatment group.

Statistical Analysis

Statistical comparisons for the studies described herein were made between groups by using the paired or unpaired t-tests, unless noted otherwise. Fisher's Exact test was used to compare VT inducibility between control and PFD treatment groups. All values are reported as means±SEM. P<0.05 was considered significant.

### Example 2

### Echocardiographic Analysis of Experimental Models

At baseline, and at 1 wk and 5 wk after infarction, a commercially available high-resolution echocardiographic system (Vevo 660, VisualSonics, Toronto, ON, Canada) equipped with a 25-MHz mechanical transducer was used for echocardiography. Rats were placed supine on a warming platform, and ECG limb electrodes were attached. To minimize ultrasound attenuation, the chests were shaved and cleaned with a chemical hair remover (Nair). Aquasonic 100 gel (Parker Laboratories, Fairfield, N.J.) was applied to the thoracic surface to optimize visibility of the cardiac chambers. Parasternal long-axis and parasternal short-axis two-dimensional views were acquired.

Using the long-axis view, left ventricular (LV) end-systolic and end-diastolic volumes (ESV and EDV), as well as LV ejection fraction (LVEF), were calculated by using frames with the maximal and minimal cross-sectional area and width. The system software utilizes a formula based on a cylindrical-hemiellipsoid model (volume=8*area2÷3÷length). LVEF was calculated using the following formula: (EDV−ESV)/EDV*100. Fractional shortening (FS) was evaluated from the M mode of the parasternal long-axis view at the papillary muscle level on the basis of the percent changes of LV end-diastolic and end-systolic diameters. LV mass was estimated using the following equation at end diastole: LV mass=1.05*(epicardial volume−endocardial volume), where volume is based on the cylindrical-hemiellipsoid model. These evaluations of LV function in the rodent are well validated. Echocardiographic acquisition and analysis were obtained while blinded to the treatment group.

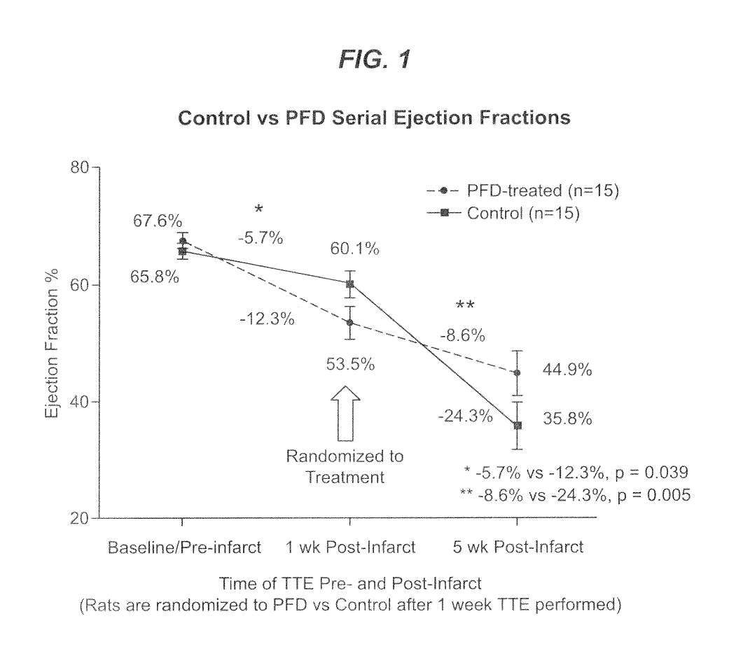

Serial echocardiography at baseline, 1 week post-MI, and 5 weeks post-MI, showed evidence of progressive LV remodeling for rats in both groups, including LV dilatation, increases in EDV and ESV, and decreases in ejection fraction. However, the pirfenidone-treated group had significantly less decline in its ejection fraction (from 68±6% to 45±14% in the control group and from 66±5% to 36±15% in the PFD treated group) (FIG. 1). During the treatment period (week 1 to week 5) there was a significantly (p=0.005) lower percent decrease in EF in the pirfenidone-treated rats (8.6%) compared to controls (24.3%).

### Example 3

### Electrophysiologic Analysis and Evaluation of Arrhythmias in Experimental Models

Optical mapping is a technique to perform high-resolution electrophysiologic evaluation of the cardiac tissue. To summarize the procedure, ten thousand simultaneous optical action potentials were recorded with a 100×100 CMOS camera within a 19 mm×19 mm mapping field on the epicardium of the LV anterior wall. Using a 1000-W tungsten-halogen light source, fluorescence was excited with an excitation filter of 530 nm and transmitted with an emission long-pass filter of >630 nm. Fluorescent optical maps were acquired at 2000 Hz during programmed electrical stimulation. Optical mapping was performed 5 wks after MI. Rats were injected with heparin (500 U ip) 15 min before excision of the heart, and were then anesthetized with pentobarbital sodium (50 mg/kg ip). After adequate anesthesia, the heart was rapidly excised and arrested by immersion in cold cardioplegia solution. The aorta was cannulated and retrogradely perfused, at a rate of 6 mL/min, with 37° C. modified Tyrode solution containing (in mmol/L): 130 NaCl, 20.0 NaHCO₃, 1.2 MgCl₂, 4.0 KCl, 5.6 glucose, and 1.8 CaCl₂, gassed with 95% 02/5% C02. Extraneous tissue was carefully removed from the heart. The cannulated heart was then placed in 37° C. Tyrode solution in a specialized temperature-controlled optical recording chamber (maintained at 37° C.) while ECG, perfusion rate, and temperature were measured continuously for the duration of the experiment. Before optical recordings, Tyrode solution containing voltage-sensitive dye PGH I (10 μL of 5 mM stock solution) was perfused through the preparation over a 5-min period.

Once a cannulated heart was perfused with PGH I, it was placed in the optical chamber with its LV anterior wall pressed against the imaging window. In order to include areas of normal, border zone, and infarct tissues within the mapping field, comparable mapping positions were used for all the hearts. During optical recordings, contractility was blocked with 15 mM butadione monoxime (BDM). Ventricular epicardium bipolar pacing, at a stimulus amplitude of 2× threshold, was performed on normal tissue near the infarct zone. Mapping was recorded during pacing drives of 250 ms to 90 ms (decremented by 10 ms), as well as during S1-S2 pacing using a basic cycle length (BCL) of 200 ms and maximum S2 of 150 ms and decremented by 10 ms. Programmed stimulation, with up to three extrastimuli, and burst pacing (from 90 ms to 60 ms) were used to assess arrhythmia inducibility. Inducibility was defined as the ability to provoke sustained (>30 s) ventricular tachycardia (VT) or ventricular fibrillation. Maps were also captured during programmed stimulation and with all episodes of arrhythmia.

Optical mapping data was analyzed using modified OMproCCD software (from Bum-rak Choi, Pittsburgh, Pa.) and Matlab custom software. Raw fluorescence data was viewed as a movie of normalized fluorescence intensity, which revealed activation within the field of view. Quantitative data was obtained from optically derived action potentials (APs) for each of the 10,000 pixels of the CMOS camera. Activation time and action potential duration at 50% (APD50) and 80% repolarization (APD80) were measured for each paced cycle length (PCL). Activation time was calculated at the maximum rate of rise of the fluorescent AP (dF/dt). APD80 is the duration from the activation time (start of the action potential) to the time point where the action potential has recovered to 20% maximal fluorescent signal (peak of the optical AP). Isochronal maps of activation were constructed for each map. Rise time was calculated as the time between takeoff and at the peak of the action potential. The OMproCCD software was used to calculate conduction vectors representing conduction velocities and conduction direction at each pixel, as previously described. Phase differences, calculated as the average difference with neighboring activation times at each site, were measured to quantify the spatial heterogeneity of conduction, as previously described. Frequency histograms were constructed for the phase differences within a recorded area. These histograms were summarized as the median phase time at 50th percentile (P50), and the 5th and 95th percentiles (P5 and P95, respectively) of the distribution. The absolute degree of heterogeneity, or heterogeneity range, was quantified as the width of the distribution, P95−P5, while heterogeneity index was defined as the heterogeneity range divided by the median phase (P95−P5)/P50. All parameters were determined for both control and PFD groups and their respective non-infarct, border, and infarct zones. These zones were identified using the amplitude map of fluorescence, as previously described and validated. Transitions from areas of high amplitude (non-infarct) to lowest amplitude (infarct) were considered border zones. Further evidence from triphenyltetrazolium chloride (TTC) staining, imaging of the heart under normal light conditions, and from fluorescence images, were also used to corroborate amplitude maps.

VT Inducibility and Electrophysiologic Characterization

The rate of VT induction was 73.3% in control MI rats, which is consistent with what has been shown in the art. The rate of VT induction for PFD animals, however, was significantly decreased, at 28.6% (p=0.027).

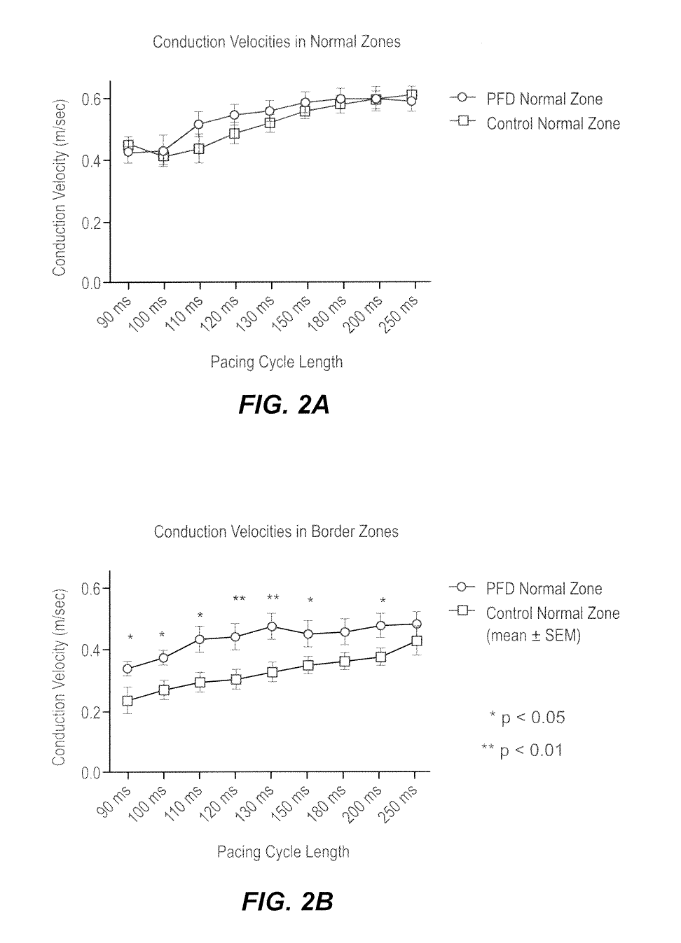

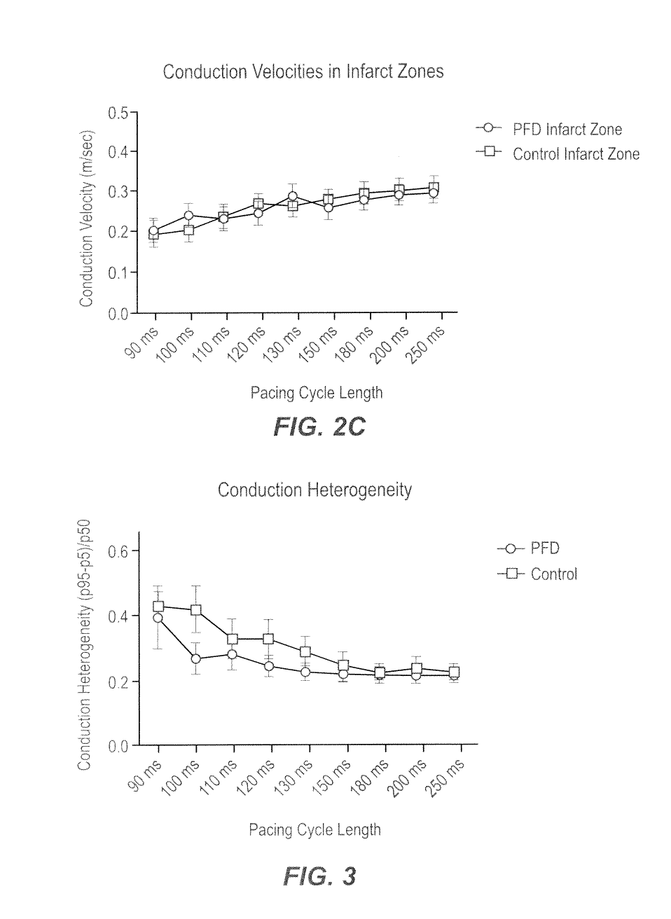

Optical mapping was used to analyze conduction action potential properties. FIG. 2 shows the conduction velocities measured in the 3 areas of the LV in all animals. Conduction velocities at all paced cycle lengths in the remote non-infarct zones of both control and PFD groups were similar between the two groups (FIG. 2). Conduction velocities in the infarct zones of both control and PFD groups were significantly slower than normal (and border zone areas) and were similar between the two groups (FIG. 2). Conduction velocities in the border zones (the area that predisposes to post-MI ventricular tachycardia) of both groups were intermediate to that of the remote non-infarct and infarct zones. However, the conduction velocities in the border zones for the PFD group were significantly faster, at all PCLs, compared to those in the border zones of control animals (p <0.05, FIG. 2).

FIG. 3 shows the conduction heterogeneity (which has been shown to be related to an increased propensity for arrhythmias) measured in both groups across all tested cycle lengths. There was a trend toward higher conduction heterogeneity indices in control animals compared to those of PFD animals (p=0.146). The difference in conduction through infarcts of similar size, for control and PFD animals were visualized in representative activation movies and showed more slowing and increased heterogeneity of conduction for the control animal. All of these parameters have previously been demonstrated to be related to enhanced substrate for ventricular arrhythmias.

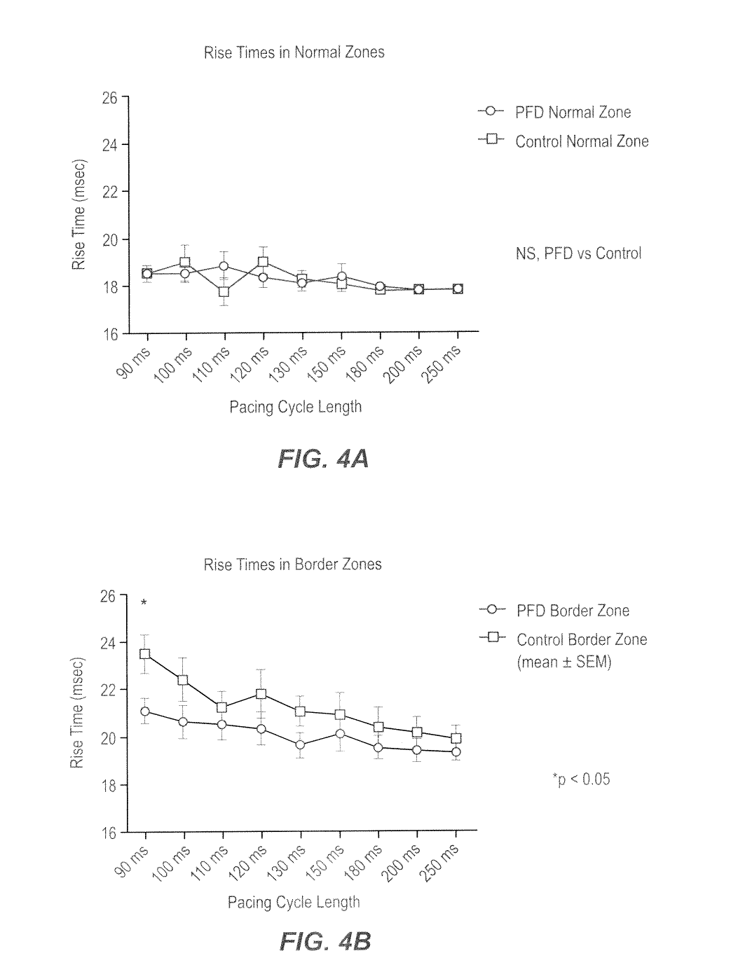

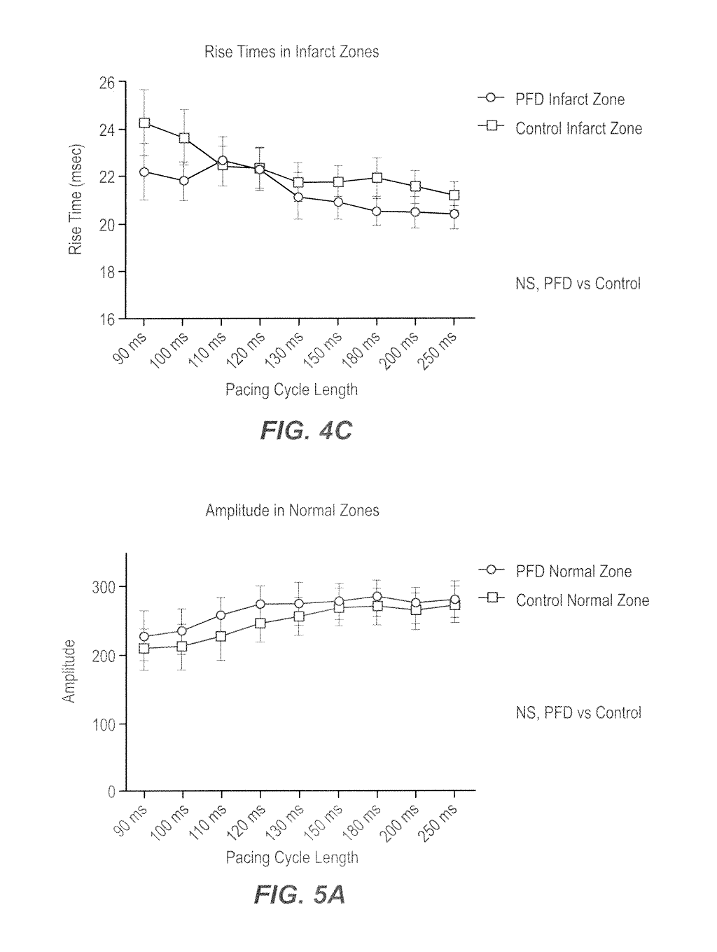

The maximal rate of AP rise (dF/dt) and rise times (duration from AP takeoff to peak of fluorescent AP) for control and PFD non-infarct zones were similar respectively, as were the rise and rise times for control and PFD infarct zones respectively. However, there was a trend, at all PCLs, for the rise of PFD border zones to be faster than the rise of control border zones. Conversely, there was a trend, at all PCLs, for the rise times of PFD border zones to be lower than those of control border zones (FIG. 4). This comparison is statistically significant at the lowest PCL tested (FIG. 4).

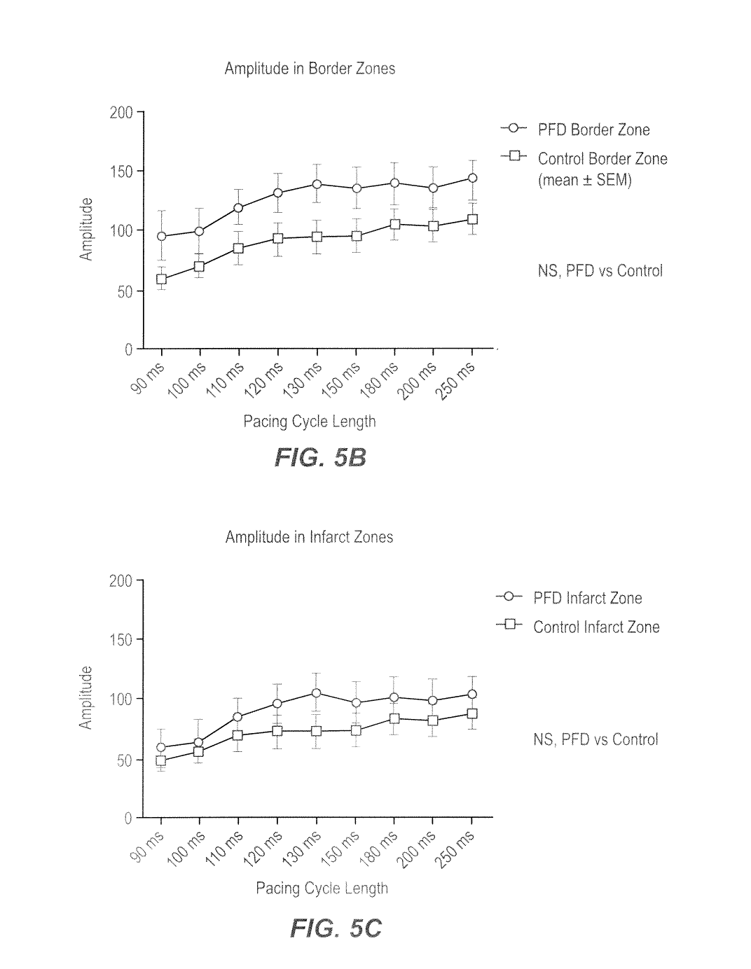

The amount of fluorescence amplitude for the three zones was also quantified, shown in FIG. 5. Normal areas had the highest amplitude, infarct areas the least, and border areas in the middle. A trend toward higher amplitudes of fluorescence in the border zones of pirfenidone-treated rats was noted, as compared to those of the controls (FIG. 5). This suggested that pirfenidone may have had an impact on infarct expansion in the border zone (decreased scar expansion), since the pirfenidone border zones likely had more viable cardiomyocytes to emit the additional fluorescence. This was validated histologically by examining infarct sizes for these hearts (see below).

### Example 4

### Histological Analysis of Infarct Size and Fibrosis

Ventricular tissue samples were fixed in 10% neutral buffered formalin. The samples were embedded in paraffin, sectioned (10-μm thick), and then stained with Masson's trichrome or Sirius red with fast green counterstain. Stained slides were examined under light microscopy, digitized using a high-resolution scanner, and analyzed using Photoshop CS software. Infarct areas on Masson's trichome corresponded tightly with areas of dense Sirius red staining with minimal to no fast green. Infarct scar area and total area of left ventricular myocardium, for all sections, were manually traced in the digital images and automatically calculated by the software. Infarct size, expressed as a percentage, was measured by dividing the sum of infarct areas from all sections by the sum of LV areas from all sections and multiplying by 100.

The total area of fibrosis was also assessed. After excluding the infarct area (defined as dense fibrosis), fibrosis in the border and non-infarct zones was quantified from digital photomicrographs of the Sirius red-stained sections. Areas containing blood vessels and perivascular interstitial cells were also excluded from fibrosis quantification. The red pixel content of digitized images relative to the total tissue area was counted by using the Adobe Photoshop CS software.

### Implications of Examples Described Above

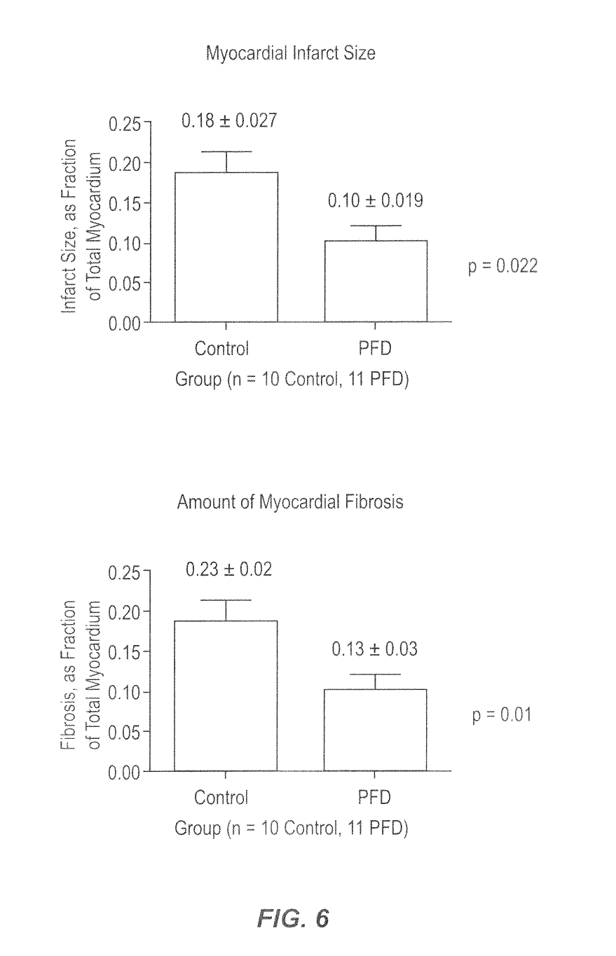

The amount of infarct fibrosis was quantified as percent of total myocardium. Controls had almost twice as large an infarct (18%±2.7%) as the PFD group (10±1.9%; p=0.022) (FIG. 6). The amount of fibrosis (including border zones and non-MI areas and infarct scar) was also less in the PFD group (13±3%), compared to controls (23±2%; p=0.01) (FIG. 6).

Previous research [Breithardt et al. Eur Heart J (1989) 10 Suppl E:. 9-18; Spach. Circ Res (2007) 101(8): 743-5; Spach et al. J Cardiovasc Electrophysiol (1994) 5(2): 182-209; Jacobson et al. Heart Rhythm (2006) 3(2): 189-97; Marchlinski et al. Circulation (2004) 110(16): 2293-8; Verheule et al. Circ Res (2004) 94(11): 1458-65] has shown that fibrosis is strongly correlated with atrial and ventricular arrhythmias. Increased fibrosis leads to decoupling of muscle fibers, conduction slowing and conduction blocks, as well as “zig-zag” and chaotic conduction. The distribution of fibrosis is also important: a finger-like distribution, as opposed to a more diffuse picture, is also thought to cause more disruption of wave propagation and is therefore more arrhythmogenic [Breithardt et al. Eur Heart J (1989) 10 Suppl E: 9-18]. After an MI, cardiac fibrosis in the infarct border zone has such a string-like distribution and is more likely to cause alterations of direction-directed electrical propagation with the fibrotic tissue interrupting normally tight cell-cell coupling. This is believed to contribute to slowing and heterogeneous conduction velocities, eventually setting up the formation of re-entrant circuits that predispose to ventricular arrhythmias. In the rodent ischemia-reperfusion model described herein, significant remodeling occurred over the course of 5 weeks post-MI. Control animals had progressive LV dilation with decreased LVEF. Fibrosis occurred not only within the infarct scar but also in the areas bordering the infarct (infarct border zone) and in normal myocardium distant to the infarct. Noninfarct fibrosis is a well-described phenomenon after an MI and is believed to contribute to deleterious remodeling (both mechanically and electrophysiologically).

The observed fibrosis, particularly in the infarct border zone, correlated with slower conduction velocities in the border zone of control animals and suggests that the fibrosis had led to electrical uncoupling. Furthermore, compared to normal myocardium, the action potential rise was lower, and its rise time was longer in the border zone of control infarcts; these findings are all consistent with slower conduction velocities and increased conduction heterogeneity. The altered and heterogeneous conduction velocities led to more inducible VT. These results are very similar to previously reported optical mapping studies for myocardial infarction in rodents, larger animals and humans.

The results highlight the role of fibrosis attenuation in the post-MI setting and its impact on LV function and VT inducibility. PFD, an antifibrotic drug, was shown to be able to decrease the amount of fibrosis in an ischemia-reperfusion rat model. This decrease in fibrosis correlated with a decrease in infarct expansion as well as with improved left ventricular function by echocardiography. Further, it was shown that decreased fibrosis was associated with decreased VT susceptibility. This was related to an improvement in conduction velocity and conduction heterogeneity, which are important contributors to the substrate for VT in the post-MI setting.

The animals undergoing ischemia-reperfusion myocardial infarction were not randomized to PFD treatment until after 1 week post-MI. Because clinical studies with anti-inflammatory agents, particularly corticosteroids, have shown adverse outcomes in the post-MI setting, one concern was that treatment so early in the post infarct period would have impaired wound healing, thus causing a weaker scar and possibly increasing mortality due to CHF or cardiac rupture. Several studies have shown that 1 week after a myocardial infarction in rodents is a safe and efficacious time frame. No increased mortality, CHF, or arrhythmias in animals treated with PFD were noted. On the contrary, and surprisingly, animals treated with PFD appeared to have less infarct expansion, improved LV function, and decreased VT susceptibility.

Pirfenidone attenuated the total amount of fibrosis, as well as extra-infarct fibrosis. Despite delaying treatment until 1 week after the MI, PFD appeared to have an effect on decreasing the infarct size, compared to control infarcts. Therefore, absent the PFD intervention, ongoing remodeling changes may actually contribute to infarct expansion long after the initial ischemic insult. There is evidence that this is indeed the case, with studies indicating that cardiomyocyte death can occur in non-infarcted myocardium, particularly within the infarct border zone, for weeks after an MI. Underlying mechanisms associated with this pathology include wall restructuring, side-to-side slippage of cells, and cardiac dilatation (Cheng, Kajstura et al. 1996; Olivetti, Capasso et al. 1990). Thus, by decreasing fibrosis, PFD improved cardiac remodeling, as evidenced by the improvement in LV function, and this likely contributed to the decrease in infarct size.

Fibrosis within the infarct border zone for PFD animals was not only decreased but its distribution appeared less heterogeneous, with less of the finger-like projections seen in control infarcts. This decrease in erratic distribution, as well as in quantity of fibrosis, was associated with improved conduction velocities in PFD border zones. A concurrent increase in action potential rise and faster rise time in PFD border zones further confirm these findings. These results, as well as decreased conduction heterogeneity, were likely responsible for the almost three-fold decrease in VT susceptibility in PFD animals.

### Example 5

### Ventricular Fibrillation Mapping

Animal Models: Twenty-four dogs weighing 25-30 Kg were divided into three groups: control (n=11), congestive heart failure (n=7), and congestive heart failure with the antifibrotic drug pirfenidone (n=6). Heart Failure (CHF) was induced in 7 dogs via four weeks of rapid ventricular pacing via a lead placed in the RV and pulse generator set to pace at 240 bpm followed by ablation of the AV node to create complete heart block, as described in Li, et al., Circulation 1999; 100:87-95. Ventricular function was monitored weekly with transthoracic echocardiography for 4 weeks. At 4 weeks, the optical mapping study was performed. Four weeks was chosen based on previous data demonstrating significant ventricular dilatation and remodeling, and decreased contractility in that time.

Heart Failure with Pirfenidone (PFD): Heart failure was induced in 6 dogs as described above and PFD was administered as described in Lee et al., Circulation 2006; 114; 1703-12. Oral PFD (800 mg 3 times per day; InterMune, Brisbane, Calif.) was started 2 days before the initiation of pacing and was discontinued >6 half-lives (24 hours) before the optical mapping study.

Optical Mapping Studies: A coronary perfused left ventricular preparation was used as described in Wu et al., J Cardiovasc Electrophysiol 1998; 9:1336-47. Briefly, following sedation with sodium pentothal (0.25 mg/Kg), a left lateral thoracotomy is performed and the heart was rapidly excised. It was then perfused with cardioplegic solution ((in mmol/L): NaCl 123, KCl 15, NaHCO₃ 22, NaH₂PO₄ 0.65, MgCl₂ 0.50, glucose 5.5, CaCl₂ 2, bubbled with 95% O₂/5% CO₂) retrogradely through the aorta. The ventricles were removed at approximately 1 cm below the AV ring and the left anterior descending coronary artery (LAD) was perfused. The right ventricle was removed and the left ventricle was cut to the size that was perfused by the LAD and included a papillary muscle. All ventricular branches were then ligated.

The ventricular preparation was then transferred to a tissue chamber maintained at 37° C. The perfusion line in the LAD was perfused with modified Tyrode's solution ((in mmol/L): NaCl 123, KCl 5.4, NaHCO₃ 22, NaH₂PO₄ 0.65, MgCl₂ 0.50, glucose 5.5, CaCl₂ 2, bubbled with 95% O₂/5% CO₂). Prior to optical recordings, a bolus of 30-40 μl of the voltage sensitive dye PGH-1 was injected directly into the perfusate.

With an optical mapping system described in Wu et al., J Cardiovasc Electrophysiol 1998; 9:1336-47, optical recordings were then made from 4-cm² area on 3 surfaces of the preparation (epicardial, endocardial (including the papillary muscle, and transmural) by a 16×16 photodiode array (C4657 Hamamatsu, Bridgewater, N.J.) that recorded 256 simultaneous optical action potentials. During optical recordings from a preparation, contractility was blocked with 15 mM 2,3-butadione monoxime (BDM; Sigma-Aldrich)11. Plunge electrodes were placed on the recording surface around the field of view for both pacing and monitoring. Two plunge electrodes were dedicated for recording a bipolar signal for monitoring the electrical activity of the preparation. VF was initiated with either extra stimuli or with rapid burst pacing at a cycle length of 50 ms, a pulse width of 9.9 ms, and an output of 9.9 mA. Several 4-s episodes of VF were recorded on each surface in each preparation. Activation movies of the VF were then viewed, and the activation patterns were determined. After termination of VF, signals were obtained during pacing at 250 ms and isochronal maps of activation were constructed to look at conduction. Activation patterns and wave-front direction during VF were determined from raw fluorescence movies (isopotential). Activation was characterized as 1) spiral (single reentrant circuit dominating the epoch), 2) focal (discrete, high frequency location of activation), 3) multiple wavefront (rapidly changing or varying wave fronts with wave-front collision), or 4) one broad wavefront (single wave-front passing through the map). VF was defined as rapid and irregular activations on the bipolar signal used for monitoring the electrical activity of the preparation.

Signal Processing and Frequency Domain Analysis: The signals obtained from the optical mapping recordings were sampled at 2,000 Hz, and for each signal the dominant frequency (DF) was determined and the organization was calculated as described Everett, et al., IEEE Trans Biomed Eng 2001; 48:969-78. Briefly, a fast Fourier transform (FFT) was calculated on the digitally filtered waveform. The data were detrended and multiplied by a Hamming window. The largest peak of the resulting magnitude spectrum was identified, and the positions of the harmonic peaks were determined on the basis of its position. The areas under the largest peak and three of its harmonic peaks were each calculated over a 1-Hz window. This produced an area under four peaks. The total area of the spectrum was calculated from 2 Hz up to but not including the fifth harmonic peak. The ratio of the power under the harmonic peaks to the total power in this range was calculated, and the resulting number was defined as the organization index (OI). The OI was theorized to represent the organization of AF for that signal at that period in time. To calculate the variance of the DFs, spatial coefficient of variance (SD/mean) of the DFs during a single episode of AF among all recording sites and temporal coefficient of variance of average DFs from among AF episodes for each mapping field within each preparation were calculated. Discrete, stable, high frequency areas were noted. Stability was defined as persistence over at least 90% of the epoch, and if it disappeared, it would return in the same location.

Cross Correlation Analysis: Spatial correlation analysis was performed on all recorded signals between all possible paired electrogram combinations in each animal. The cross-correlation function was calculated at zero lag for each electrogram combination, and the peak value was considered the correlation coefficient, representing the degree of correlation between the two signals. All of the correlation coefficients calculated from an AF recording with optical mapping were then averaged to produce a mean correlation value for each AF episode.

Statistical Analysis: Data were expressed as the mean±DF. For comparisons among all mapping analysis variables, a range of mixed effects models was used. The models employed dog-specific (independently and identically distributed) random effects to account for the repeated measures made on a dog both within and across recording locations. Various contrasts (sub-models of the overall model) were explored to determine the importance of the study groups, recording site, and the group by recording site interaction effects. These contrasts were tested with a Chi-squared likelihood ratio test in a nested model fashion. Statistical significance was defined as p<0.05.

VF Activation Patterns: On examination of the optical mapping activation sequences, 4 types of activation patterns were seen—spiral wave, focal area of activation, multiple waves, and on broad wavefront sweeping through the field of view. Table 2 shows the types of activation patterns that were seen on each mapped surface for each dog.

TABLE 2

| | VF Activation Patterns | | | Stable High DF | | |
|---|---|---|---|---|---|---|
| Dog | Epicardial | Endocardial | Transmural | Epicardial | Endocardial | Transmural |
| Control Dog 1 | broad wavefront | spiral wave | | | | |
| Control Dog 2A | broad wavefront | multiple wave | | | | |
| Control Dog 2B | multiple wave | broad wavefront | | | | |
| Control Dog 3 | focal | spiral wave | focal | X | | X |
| Control Dog 4A | multiple wave | broad wavefront | broad wavefront | | | |
| Control Dog 4B | | | focal | | | X |
| Control Dog 5 | multiple wave | | | | | |
| Control Dog 6 | multiple wave | multiple wave | focal | | | |
| Control Dog 7 | broad wavefront | | | | | |
| Control Dog 8 | focal | | | X | | |
| Control Dog 9 | broad wavefront | broad wavefront | | | | |
| CHF Dog 1 | spiral wave | | spiral wave | X | | X |
| CHF Dog 2 | broad wavefront | | | | | |
| CHF Dog 3 | multiple wave | spiral wave | focal | | X | X |
| CHF Dog 4 | focal | multiple wave | focal | X | | X |
| CHF Dog 5 | broad wavefront | focal | spiral wave | | X | X |
| CHF Dog 6 | | spiral wave | | | X | |
| CHF Dog 7 | multiple wave | multiple wave | focal | | | X |
| PFD Dog 1 | multiple wave | | spiral wave | | | X |
| PFD Dog 2 | multiple wave | multiple wave | | | | |
| PFD Dog 3 | focal | multiple wave | multiple wave | X | | |
| PFD Dog 4 | multiple wave | multiple wave | multiple wave | | | |
| PFD Dog 5 | multiple wave | broad wavefront | | | | |
| PFD Dog 6 | focal | multiple wave | spiral wave | X | | X |

Epicardial Surface: For the Control group, only 2 of the 10 mapped epicardial surfaces showed evidence of focal activation. These two surfaces also corresponded to having stable, high DF areas. All others had activation patterns of either multiple wavelets or one broad wavefront dominating the field of view. The activation map, during pacing at 250 ms, shows homogeneous conduction throughout the field of view. Similar results were seen in the CHF and PFD groups. Both groups had 2/6 mapped surfaces having either focal activation or a spiral wave (1 CHF dog). These types of activation corresponded to stable, high DF areas. All other dogs had either multiple wavefronts or one broad wavefront dominating the field of view. These activation patterns had either transient DFu (multiple wavefronts) or the area was dominated one DF (broad wavefront). The activation images show homogeneous conduction, similar to Control, but at a slower conduction velocity.

Endocardial Surface: Mapping of the endocardial surface included the papillary muscle and only the CHF group had AF characterized by stable, high DF areas that correlated to spiral waves or focal activation patterns. Three of the five mapped endocardial surfaces in the CHF group fell into this category. Even though 2 of 7 endocardial surfaces in the Control group had activation characterized by spiral waves, no discrete, stable DFs were observed. The other 5 Controls and all of the mapped endocardial surfaces in the PFD group had either multiple or broad wavefront activation. All of the groups showed heterogeneous conduction marked by conduction slowing. This is in contrast to the homogenous conduction seen on the epicardial surface.

Transmural Surface: The transmural surface had the highest percentage of spiral wave and focal activation when compared to the other mapped surfaces for all groups. In the CHF group, the transmural surface was mapped in 5 dogs and all of them had VF activation patterns of either a spiral wave or focal activation. The VF was characterized by stable, discrete, high DF areas. In the PFD group, 50% of the mapped transmural surfaces had an activation pattern of a spiral wave that correlated to stable high DF areas. In the Control group, 75% of the transmural surfaces had focal activation. One of these did not correlate to stable, high DF areas. Each group showed heterogeneous conduction characterized by areas of conduction slowing and block.

Dominant Frequencies: Frequency domain analysis was used as a method to quantify the activation patterns that were recorded during VF. Table 2 shows where the stable, discrete high DF areas were seen. Six of 7 CHF dogs had at least one surface with a stable, high DF area. In this group, all of the transmural surfaces that were mapped had VF characterized by a discrete, stable high DF area. Only 3 of the 11 Controls and 3 of 6 PFD dogs had at least one surface with VF that was characterized by high DF areas. The epicardial surface of the control group had a VF mechanism of multiple wavefronts. High DF areas were noted in some examples, but these were not stable. Both the endocardial and transmural surfaces had VF characterized by one broad wavefront sweeping through the field of view. The corresponding DF maps are characterized by a single DF. For the CHF group, the epicardial surface had VF characterized by a broad wavefront, and the corresponding DF map was dominated by a singular DF. The endocardial and transmural surfaces both had VF characterized by stable, high DF areas. The VF mechanisms that these DF corresponded to were a focal mechanism on the endocardial surface and a spiral wave on the transmural surface. For the PFD group, a spiral wave was seen in the transmural surface and the corresponding DF map had a stable, high DF area. A focal mechanism was seen in the epicardial surface which resulted in a high DF area. The endocardial surface had VF characterized by multiple wavefronts and only transient DF areas were seen. Summary DF data is listed in Table 3. From the statistical analysis, only the coefficient of variance for temporal and spatial DFs had significant group and surface effects.

TABLE 3

| Model | Surface | Mean DF (Hz) | Max DF (Hz) | DF Spatial CoV | DF Temporal CoV |
|---|---|---|---|---|---|
| Control | Epicardium | 8.90 ± 2.54 | 10.68 ± 2.57 | 0.14 ± 0.06 | 0.05 ± 0.03 |
| | Endocardium | 8.54 ± 2.60 | 11.35 ± 2.32 | 0.07 ± 0.09 | 0.01 ± 0.01‡* |
| | Transmural | 9.06 ± 0.59 | 11.17 ± 1.89 | 0.11 ± 0.08 | 0.06 ± 0.05 |
| CHF | Epicardium | 8.23 ± 1.62 | 9.96 ± 2.13 | 0.12 ± 0.06 | 0.06 ± 0.03 |
| | Endocardium | 8.42 ± 2.04 | 10.90 ± 2.97 | 0.15 ± 0.06 | 0.07 ± 0.04† |
| | Transmural | 8.99 ± 2.00 | 14.22 ± 3.64 | 0.20 ± 0.04 | 0.08 ± 0.02 |
| PFD | Epicardium | 9.17 ± 1.08 | 10.92 ± 0.98 | 0.09 ± 0.04 | 0.05 ± 0.01 |
| | Endocardium | 8.29 ± 0.68 | 10.44 ± 1.32 | 0.11 ± 0.04 | 0.07 ± 0.03‡† |
| | Transmural | 7.93 ± 1.52 | 10.24 ± 1.89 | 0.14 ± 0.06 | 0.08 ± 0.02 |

†p < 0.001 vs Control
*p < 0.02 vs the transmural surface of that group
‡p < 0.01 vs the epicardial surface of that group

Organization and Cross Correlation Analysis: To further analyze the spatiotemporal organization of the VF recorded on each of the surfaces of each of the models, the organization index (OI) was used to measure the organization of the recordings by quantifying the differences in the resulting FFTs. Summary data from OI maps are shown in Table 4. As the table shows, the Control group had higher mean and maximum OI values than either the CHF or PFD groups. These differences reached significance in the endocardial surface. Within goups, the endocardial surface of the control group had higher OI levels than either the epicardial or transmural surfaces. In the PFD group, the endocardial surface had the lowest OI levels and this reached significance when compared to the transmural surface. The Control group also showed the most temporal stability in OI levels as this group had the lowest OI temporal CoV values at all surfaces with the lowest measurements found on the endocardial surface. The endocardial and transmural surfaces of the CHF and PFD groups were significantly different than those of the Control group.

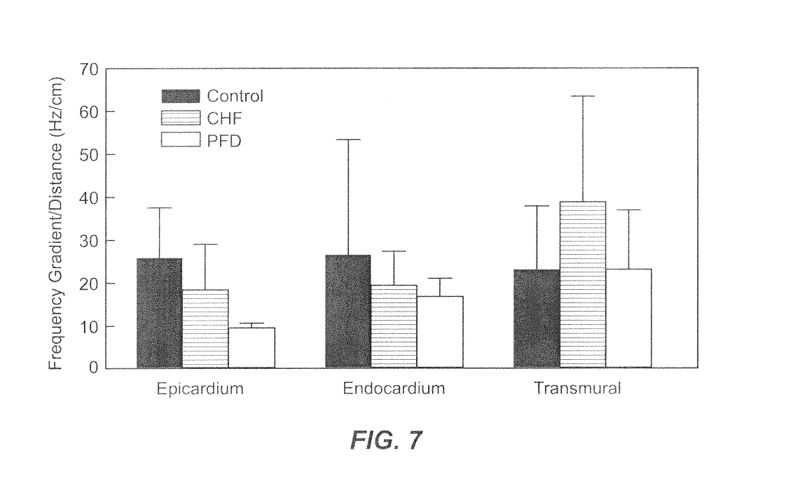

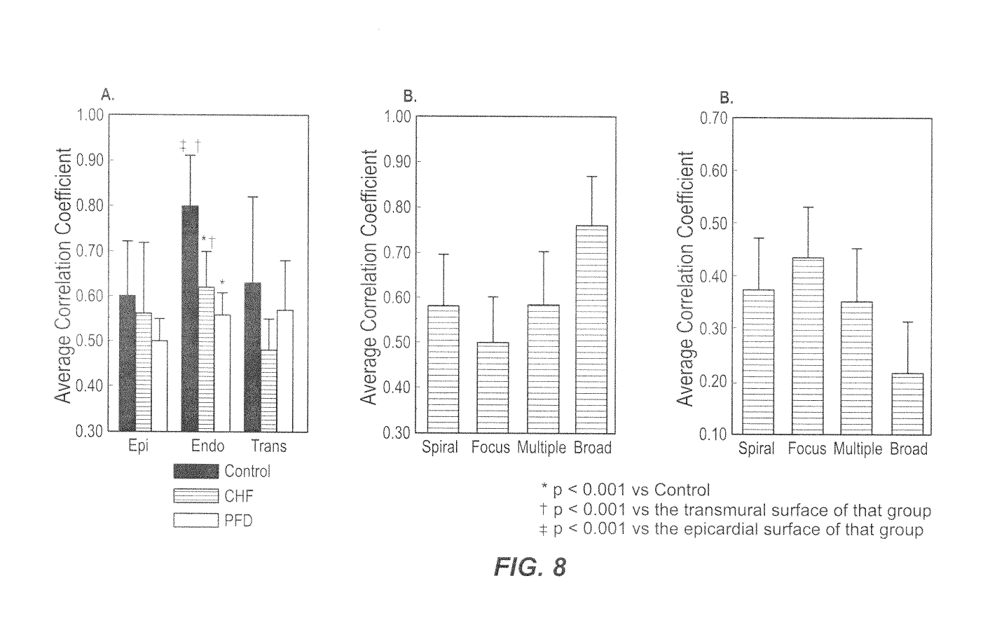

For each VF episode, all possible pairs of signals were cross-correlated, and the average correlation coefficients for each surface of each group is shown in FIG. 8A. FIG. 7 shows the gradient of frequencies over distance across the endocardial surface, transmural surface and epicardial surface. Pirfendidone preserved the transmural gradient to that similar to control animals, whereas untreated animals with heart failure have a very large gradient.

TABLE 4

| Model | Surface | Mean OI | Max OI | OI Spatial CoV | OI Temporal CoV |
|---|---|---|---|---|---|
| Control | Epicardium | 0.50 ± 0.12 | 0.68 ± 0.11 | 0.17 ± 0.04 | 0.11 ± 0.04 |
| | Endocardium | 0.63 ± 0.08‡* | 0.77 ± 0.10‡* | 0.13 ± 0.05 | 0.05 ± 0.03‡* |
| | Transmural | 0.53 ± 0.14 | 0.70 ± 0.10 | 0.17 ± 0.03 | 0.09 ± 0.05 |
| CHF | Epicardium | 0.49 ± 0.12 | 0.65 ± 0.06 | 0.16 ± 0.03 | 0.12 ± 0.03 |
| | Endocardium | 0.46 ± 0.09† | 0.63 ± 0.09† | 0.17 ± 0.02 | 0.13 ± 0.04† |
| | Transmural | 0.43 ± 0.07 | 0.60 ± 0.08 | 0.19 ± 0.04 | 0.13 ± 0.01† |
| PFD | Epicardium | 0.44 ± 0.05 | 0.62 ± 0.05 | 0.17 ± 0.01 | 0.14 ± 0.01 |
| | Endocardium | 0.45 ± 0.05†* | 0.61 ± 0.05†* | 0.16 ± 0.03 | 0.13 ± 0.01† |
| | Transmural | 0.48 ± 0.05 | 0.65 ± 0.06 | 0.17 ± 0.03 | 0.13 ± 0.01† |

†p < 0.001 vs Control
*p < 0.02 vs the transmural surface of that group
‡p < 0.01 vs the epicardial surface of that group

### Examples of Embodiments of the Invention Include

1. A method of treating a patient who has suffered an acute myocardial infarction (AMI) comprising administering to the patient a therapeutically effective dose of a therapeutic having an anti-fibrotic effect,

wherein optionally the treatment is initiated at a time period about to 42 days after suffering the AMI, and optionally continues for up to 3 to 6 months.

2. The method of paragraph 1, wherein the method is to limit expansion of an infarct scar due to the AMI.

3. The method of paragraph 1, wherein the treatment is initiated about 5-10 days after the AMI.

4. The method of paragraph 3, wherein the treatment is initiated about 7 days after the AMI.

5. The method of any one of paragraphs 1-4, wherein the treatment is for at least 2 weeks.

6. A method of reducing the incidence of congestive heart failure in a patient who suffered an acute myocardial infarction (AMI), comprising administering to the patient a therapeutically effective dose of a therapeutic having an anti-fibrotic effect,

wherein the therapeutically effective dose reduces the incidence of congestive heart failure.

7. The method of paragraph 6, wherein the patient is at an increased risk of congestive heart failure due to the AMI.

8. The method of paragraph 6 or 7, wherein the treatment is initiated about 1 to 42 days after the suffering of the AMI.

9. A method of preserving viable cardiac tissue or controlling or reducing myocardial infarct size in a patient who has suffered an acute myocardial infarction (AMI) comprising administering to the patient a therapeutically effective dose of a therapeutic having an anti-fibrotic effect,

wherein the administering of the therapeutic to the patient results in a relatively reduced infarct size on average compared to infarct size in a patient who has not been administered the therapeutic.

10. The method of paragraph 9, wherein the administering is initiated 1-42 days after suffering the AMI.

11. The method of paragraph 9 or 10, wherein the relative reduction in infarct size is at least 5%.

12. A method of reducing the incidence of ventricular tachycardia in a patient in need thereof, comprising administering to the patient a therapeutically effective dose of a therapeutic having an anti-fibrotic effect,

wherein the administering of the therapeutic prevents or reduces the incidence of ventricular tachycardia.

13. The method of paragraph 12, wherein the patient has suffered an acute myocardial infarction (AMI).

14. The method of paragraph 13, wherein the administering is initiated about 1 to 42 days after the suffering of the AMI.

15. The method of paragraph 14, wherein the administering is initiated about 7 days after the suffering of the AMI.

16. A method of treating or preventing ventricular fibrillation in a patient in need thereof, comprising administering to the patient a therapeutic having an anti-fibrotic effect,

wherein the administering of the therapeutic prevents ventricular fibrillation in the patient.

17. The method of paragraph 16, wherein the patient has suffered an acute myocardial infarction (AMI).

18. The method of paragraph 17, wherein the administration is initiated about 1 to 42 days after the suffering of the AMI.

19. The method of paragraph 18, wherein the administration is initiated about 7 days after the suffering of the AMI.

20. The method of any one of paragraphs 16-19, wherein the administering reduces the incidence of sudden cardiac death.

21. The method of any one of paragraphs 16-20, wherein the administering reduces cardiac risk of the patient.

22. A method of controlling arrhythmia in a patient in need thereof, comprising administering to the patient a therapeutic having an anti-fibrotic effect,

wherein the administering of the therapeutic controls arrhythmia in the patient.

23. The method of paragraph 22, wherein the patient has suffered an acute myocardial infarction (AMI).

24. The method of paragraph 23, wherein the administration is initiated about 1 to 42 days after the suffering of the AMI.

25. The method of paragraph 24, wherein the administration is initiated about 7 days after the suffering of the AMI.

26. The method of any one of paragraphs 22-25, wherein the administering treats ventricular remodeling.

27. The method of any one of paragraphs 1-26, wherein the patient had not previously suffered an AMI.

28. The method of any one of paragraphs 1-27, wherein the therapeutic having an anti-fibrotic effect is a therapeutic that

reduces tissue remodeling or fibrosis,

reduces the activity of transforming growth factor-beta (TGF-β), targets one or more TGF-β isoforms, inhibits TGF-β receptor kinases TGFBR1 (ALK5) and/or TGFBR2, or modulates one or more post-receptor signaling pathways;

is an endothelin receptor antagonists, targets both endothelin receptor A and endothelin receptor B or selectively targets endothelin receptor A;

reduces activity of connective tissue growth factor (CTGF);

inhibits matrix metalloproteinase;

reduces the activity of epidermal growth factor (EGF), targets the EGF receptor, or inhibits EGF receptor kinase;

reduces the activity of platelet derived growth factor (PDGF), targets PDGF receptor (PDGFR), inhibits PDGFR kinase activity, or inhibits post-PDGF receptor signaling pathways;

reduces the activity of vascular endothelial growth factor (VEGF), targets one or more of VEGF receptor 1 (VEGFR1, Flt-1), VEGF receptor 2 (VEGFR2, KDR), the soluble form of VEGFR1 (sFlt) and derivatives thereof which neutralize VEGF, inhibits VEGF receptor kinase activity;

inhibits multiple receptor kinases such as BIRB-1120 which inhibits receptor kinases for vascular endothelial growth factor, fibroblast growth factor, and platelet derived growth factor;

interferes with integrin function;

interferes with pro-fibrotic activities of IL-4 and IL-13, targets IL-4 receptor, IL-13 receptor, the soluble form of IL-4 receptor or derivatives thereof;

modulates signaling though the JAK-STAT kinase pathway;

interferes with epithelial mesenchymal transition, inhibits mTor;

reduces levels of copper;

reduces oxidative stress;

inhibits prolyl hydrolase;

inhibits phosphodiesterase 4 (PDE4) or phosphodiesterase 5 (PDE5), or

modifies the arachidonic acid pathway.

29. The method of any one of paragraphs 1-28, wherein the therapeutic is pirfenidone or compound of formula (I), (II), (III), (IV), or (V) or a pharmaceutically acceptable salt, ester, solvate, or prodrug thereof:

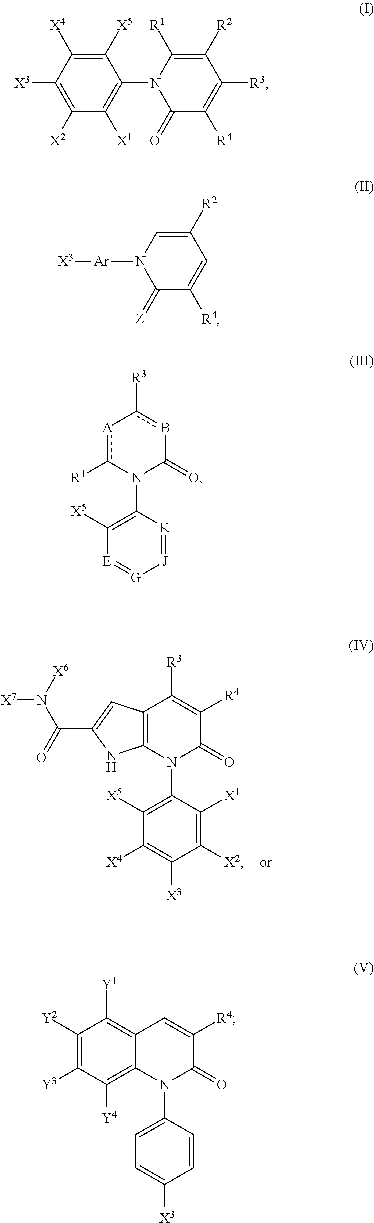

wherein

A is N or CR²; B is N or CR⁴; E is N or CX⁴; G is N or CX³; J is N or CX²; K is N or CX¹; a dashed line is a single or double bond,

R¹, R², R³, R⁴, X¹, X², X³, X⁴, X⁵, Y¹, Y², Y³, and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted C₁-C₁₀ alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, substituted C₁-C₁₀ alkoxyalkyl, C₁-C₁₀ carboxy, substituted C₁-C₁₀ carboxy, C₁-C₁₀ alkoxycarbonyl, substituted C₁-C₁₀ alkoxycarbonyl, CO-uronide, CO-monosaccharide, CO-oligosaccharide, and CO-polysaccharide;

X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and

Ar is pyridinyl or phenyl; and Z is O or S.

30. The method of any one of paragraphs 1-29, wherein a therapeutically effective amount of pirfenidone or a pharmaceutically acceptable salt, ester, solvate, or prodrug thereof is administered to the patient.

31. The method of any one of paragraphs 1-29, wherein the therapeutic administered to the patient comprises a compound of formula (II)

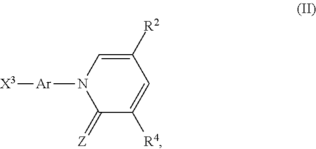

wherein

X³ is H, OH, or C₁₋₁₀alkoxy, Z is O, R² is methyl, C(═O)H, C(═O)CH₃, C(═O)O— glucosyl, fiuoromethyl, ditluoromethyl, tritluoromethyl, methylmethoxyl, methylhydroxyl, or phenyl; and R⁴ is H or hydroxyl,

or a salt, ester, solvate, or prodrug thereof.

32. The method of paragraph any one of paragraphs 1-29, wherein the therapeutic administered to the patient is selected from the group consisting of

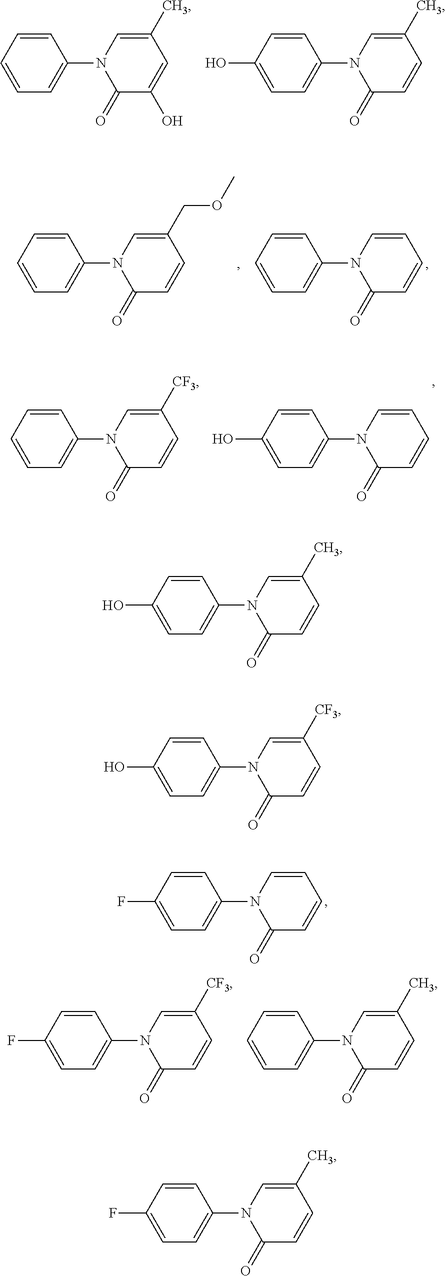

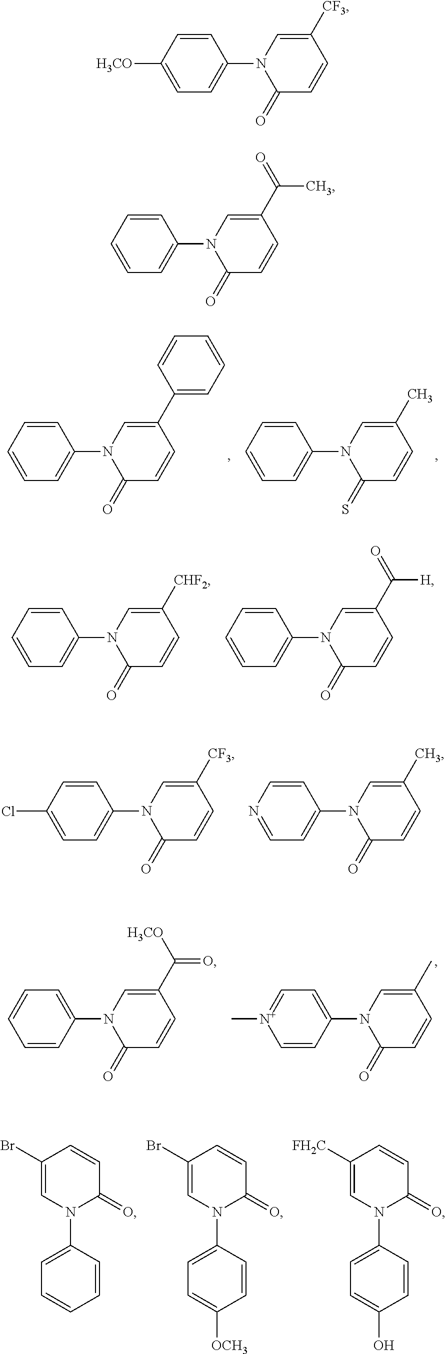

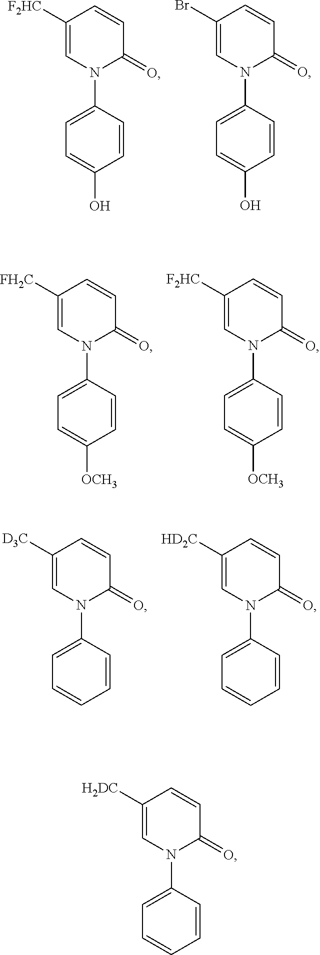

a compound as listed in Table 1,
and pharmaceutically acceptable salts, esters, solvates, and prodrugs thereof

33. The method of any one of paragraphs 1-28, wherein the therapeutic is a compound of formula (I), (II), (III), (IV), or (V) or a pharmaceutically acceptable salt, ester, solvate, or prodrug thereof:

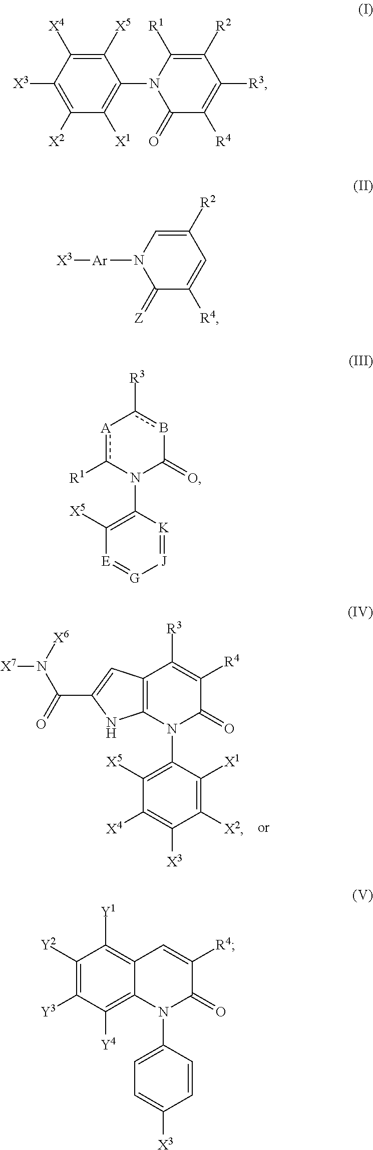

wherein
A is N or CR²; B is N or CR⁴; E is N, N⁺X⁴ or CX⁴; G is N, N⁺X³ or CX³; J is N, N⁺X² or CX²; K is N, N⁺X¹ or CX¹; a dashed line is a single or double bond,

R¹, R², R³, R⁴, X¹, X², X³, X⁴, X⁵, Y¹, Y², Y³, and Y⁴ are independently selected from the group consisting of H, deuterium, optionally substituted C₁-C₁₀ alkyl, optionally substituted C₁-C₁₀ deuterated alkyl, optionally substituted C₁-C₁₀ alkenyl, optionally substituted C₁-C₁₀ thioalkyl, optionally substituted C₁-C₁₀ alkoxy, optionally substituted cycloalkyl, optionally substituted heterocycloalkyl, optionally substituted heteroalkyl, optionally substituted aryl, optionally substituted heteroaryl, optionally substituted amido, optionally substituted sulfonyl, optionally substituted amino, optionally substituted sulfonamido, optionally substituted sulfoxyl, cyano, nitro, halogen, hydroxyl, SO₂H₂, optionally substituted C₁-C₁₀ alkoxyalkyl, optionally substituted C₁-C₁₀ carboxy, optionally substituted C₁-Cₜₒ alkoxycarbonyl, CO-uronide, CO-monosaccharide, CO-oligosaccharide, and CO-polysaccharide;

X⁶ and X⁷ are independently selected from the group consisting of hydrogen, optionally substituted aryl, optionally substituted heteroaryl, optionally substituted cycloalkyl, optionally substituted heterocycloalkyl, optionally substituted alkylenylaryl, optionally substituted alkylenylheteroaryl, optionally substituted alkylenylheterocycloalkyl, optionally substituted alkylenylcycloalkyl, or X⁶ and X⁷ together form an optionally substituted 5 or 6 membered heterocyclic ring; and

Ar is optionally substituted pyridinyl or optionally substituted phenyl; and Z is O or S.

34. The method of any one of paragraphs 1-33, wherein the therapeutic is combined with a pharmaceutically acceptable carrier.

35. The method of any one of paragraphs 1-34, wherein the administering is oral.

36. The method of any one of paragraphs 1-35, wherein the therapeutically effective amount is a total daily dose of about 50 mg to about 2400 mg of the therapeutic or a pharmaceutically acceptable salt, ester, solvate, or prodrug thereof.

37. The method of paragraph 36, wherein the therapeutically effective amount is administered in divided doses three times a day or two times a day, or is administered in a single dose once a day.

38. The method of any one of paragraphs 1-37, wherein the patient is human.

What is claimed is:

1. A method of treating a patient who has suffered an acute myocardial infarction (AMI) comprising administering to the patient a therapeutically effective dose of pirfenidone or salt thereof, wherein the pirfenidone treatment is initiated at a time period of 1 to 14 days after the patient has suffered the AMI.

2. The method of claim 1, wherein the pirfenidone treatment is initiated at a time period of 3 to 14 days after the patient has suffered the AMI.

3. The method of claim 2, wherein the pirfenidone treatment is initiated at a time period of 7 days after the patient has suffered the AMI.

4. The method of claim 1, wherein the pirfenidone treatment continues up to 6 months.

5. The method of claim 1, wherein the pirfenidone treatment continues up to 3 months.

6. The method of claim 1, wherein the pirfenidone treatment reduces ventricular remodeling.

7. The method of claim 1, wherein the pirfenidone treatment reduces the incidence of one or more of ventricular arrhythmias, congestive heart failure, and sudden cardiac death.

8. The method of claim 1, wherein the pirfenidone treatment controls or reduces infarct size.

9. The method of claim 1, wherein the patient has not suffered or does not suffer from chronic myocardial infarction.

10. The method of claim 2, wherein the therapeutically effective dose of pirfenidone or salt thereof is 1 mg/kg to 60 mg/kg per day.

11. A method of reducing the incidence of congestive heart failure in a patient who has suffered an acute myocardial infarction (AMI), comprising administering to the patient a therapeutically effective dose of pirfenidone or salt thereof.

12. The method of claim 11, wherein the pirfenidone administration is initiated at a time period of 1 to 42 days after the patient has suffered the AMI.

13. The method of claim 11, wherein the pirfenidone administration is initiated at a time period of 3 to 14 days after the patient has suffered the AMI.

14. The method of claim 13, wherein the therapeutically effective dose of pirfenidone or salt thereof is 1 mg/kg to 60 mg/kg per day.

15. The method of claim 12, wherein the patient has not suffered or does not suffer from chronic myocardial infarction.

16. A method of controlling or reducing ventricular remodeling in a patient who has suffered an acute myocardial infarction (AMI) comprising administering to the patient a therapeutically effective dose of pirfenidone or salt thereof, wherein the pirfenidone administration is initiated at a time period of 1 to 14 days after the patient has suffered the AMI.

17. The method of claim 16, wherein the pirfenidone administration is initiated at a time period of 3 to 14 days after the patient has suffered the AMI.

18. The method of claim 16, wherein the pirfenidone administration is initiated at a time period of 7 days after the patient has suffered the AMI.

19. The method of claim 18, wherein the therapeutically effective dose of pirfenidone or salt thereof is 1 mg/kg to 60 mg/kg.

20. The method of claim 16, wherein the patient has not suffered or does not suffer from chronic myocardial infarction.
